(19) United States
(12) Patent Application Publication
(10) Pub. No.: US 2026/0234193 A1
(43) Pub. Date: Aug. 13, 2026

(54) INTERLEUKIN-17 PEPTIDOMIMETIC INHIBITORS

(71) Applicant: Pepticom Ltd., Jerusalem (IL)

(72) Inventors: Immanuel LERNER, Jerusalem (IL); Amit MICHAELI, Jerusalem (IL); Alexandra VARDI KILSHTAIN, Beer Sheva (IL); Victoria KOZOKARO, Netanya (IL); Shaul Yoseph LERNER, Kibutz Gezer (IL); Guila ABITBOL, Jerusalem (IL); Dikla Geula HAHAM, Jerusalem (IL); Maayan ELIAS ROBICSEK, Modiin-Maccabim-Reut (IL); Gideon BAR, Aviezer (IL)

(21) Appl. No.: 19/637,520

(22) Filed: Apr. 2, 2026

Related U.S. Application Data
(63) Continuation-in-part of application No. PCT/IL2024/050971, filed on Oct. 1, 2024.
(60) Provisional application No. 63/588,728, filed on Oct. 8, 2023.

(51) Int. Cl. C07K 7/64; A61K 38/00; A61P 17/06

(52) U.S. Cl. CPC C07K 7/64; A61P 17/06; A61K 38/00

(57) ABSTRACT

The present disclosure relates to cyclic peptidomimetic IL-17 inhibitors, to pharmaceutical compositions comprising them and to their use for treatment of immunological and inflammatory diseases and disorders.

### CROSS-REFERENCE TO RELATED APPLICATIONS

This application is a Continuation-in-part of International Application No. PCT/IL2024/050971 filed Oct. 1, 2024, which claims priority to U.S. Provisional Patent Application No. 63/588,728 filed on Oct. 8, 2023, the disclosure of each of which is incorporated herein, in its entirety, by this reference.

### TECHNICAL FIELD

The present disclosure is in the fields of peptide biochemistry and immunology and generally relates to anti-inflammatory compounds, and in particular to IL-17 inhibitors.

### BACKGROUND

The interleukin-17 (IL-17) family of cytokines play a crucial role in the immune system and are involved in various inflammatory responses. The IL-17 family consists of six members, namely IL-17A, IL-17B, IL-17C, IL-17D, IL-17E (also called IL-25), and IL-17F. IL-17 proteins are primarily produced by a group of immune cells called T-helper 17 (Th17) cells, but they can also be secreted by other cell types, including T-cells, natural killer (NK) cells, and certain innate immune cells.

IL-17A and IL-17F are prominent members of the IL-17 family. They are homodimeric cytokines, each encoded by a separate gene, but sharing significant structural and functional similarity. An IL-17AF heterodimer can also be formed. The biological activity of IL-17 is exerted by binding to a receptor complex known as the IL-17 receptor (IL-17R), which is expressed on various cell types throughout the body. The binding of IL-17 to its receptor triggers a signaling cascade that leads to the activation of several pathways involved in inflammation, including the production of pro-inflammatory cytokines, chemokines, and other mediators that recruit and activate immune cells at the site of inflammation, thus aiding in the clearance of pathogens. In the case of IL-17A and IL-17F, the signaling cascade is initiated through the assembly of a ternary complex with the IL-17RA and IL-17RC receptors.

While IL-17 is crucial for protective immune responses against certain pathogens, dysregulated or excessive IL-17 production has been implicated in various autoimmune and inflammatory conditions, such as rheumatoid arthritis, psoriasis, multiple sclerosis, and inflammatory bowel disease. Therefore, there has been significant interest in developing therapeutics that can inhibit the binding of IL-17 to its receptor and dampen the pathogenic inflammatory response associated with IL-17.

One approach to inhibiting IL-17 signaling is through the use of monoclonal antibodies (mAbs) that specifically target the IL-17 pathway. For example, the mAbs approved for the treatment of psoriasis and psoriatic arthritis include Secukinumab and ixekizumab that target IL-17A, bimekizumab that targets both IL-17A and IL-17F isoforms, and brodalumab that targets IL-17R.

However, the disadvantages of the mAbs, including non-oral administration, poor tissue penetration, lacking blood-brain barrier penetration, and relatively long half-life, limit the applications thereof. Thus, research is performed to discover potent small molecules, such as peptides, which effectively target the IL-17/IL-17R interaction to modulate immune responses. Small-molecule modulators are highly suitable for oral administration, as well as allowing for topical therapy. Furthermore, the small-molecule compounds allow a flexible treatment regimen and may be safer for use due to having faster withdrawal rate of the drug in the case of adverse events.

Liu et al., (Inhibiting complex IL-17A and IL-17RA interactions with a linear peptide, 2016, Sci Rep., 6:26071), disclose a high affinity IL-17A peptide antagonist (HAP) of 15 residues, identified through phage-display screening followed by saturation mutagenesis optimization and amino acid substitutions. HAP binds specifically to IL-17A and inhibits the interaction of the cytokine with its receptor, IL-17RA, thereby blocking the production of multiple inflammatory cytokines. HAP binds the IL-17A dimer, and the N-terminal portions of HAP form a β-strand that inserts between two IL-17A monomers while the C-terminal section forms an a helix that directly blocks IL-17RA from binding to the same region of IL-17A.

Zhang and Domling (Small molecule modulators of IL-17A/IL-17RA: a patent review (2013-2021), 2022, Expert Opinion on Therapeutic Patents, 32:11, 1161-1173), review the landscape of the patent applications which have been filed for small-molecule modulators for the treatment of IL-17A-related diseases. Some of the small molecules are at different stages of clinical trials. For example, UCB Biopharma SRL reported spirocyclic and imidazole derivatives as novel modulators of IL-17 in several patents. Hitgen Inc. also disclosed a series of imidazole derivatives for the treatment of IL-17A-related conditions in other patents. One of the patents covers small molecules antagonizing IL-17 activation that are built on a central bis amide motif scaffold or on a bioisostere scaffold, which seemingly share a binding mode to the interface of the IL-17A dimer involving four hydrogen bonds of the bis amide to the backbone of Leu97A and Leu97B.

There is an unmet need to develop novel inhibitors with improved efficacy, bioavailability and safety profiles for managing IL-17 associated autoimmune and inflammatory diseases.

### SUMMARY OF THE INVENTION

The present invention provides novel peptidomimetics, which are effective inhibitors of the interaction between interleukin 17 (IL-17) and IL-17 receptor (IL-17R). In an effort to find a small peptide-based molecule which is potentially effective in treatment of IL-17 associated diseases, a myriad of peptidomimetics, including non-naturally occurring amino acids, were designed in-silico and scanned using a dedicated algorithm. From the scanned structures, a selected cluster of peptidomimetics were synthesized and tested for inhibiting the interaction between IL-17 protein members, IL-17A, IL-17F and IL-17AF and IL-17R.

The present invention is based in part on the unexpected finding that a set of cyclic penta-peptidomimetics comprising 5 amino acid residues (hereinafter “amino acids”), wherein at least one is an artificial amino acid having a cyclic group, and optionally including at least one amino acid comprising an aromatic moiety or side-chain, were found to be effective in inhibiting the interaction of IL-17 with IL-17R. It was further found that the inhibition persisted in vivo, significantly ameliorating psoriasis symptoms in an animal model. This provides positive prospects for utilizing peptidomimetics according to the invention for effective treatment of patients suffering from IL-17 associated diseases, such as autoimmune and inflammatory diseases, for treatment of skin diseases and disorders and for use in cosmetic applications.

Thus, according to one aspect of the present invention, there is provided a peptidomimetic that inhibits the interaction between IL-17 and IL-17R, the peptidomimetic comprising 5 amino acids (position 1 is the N-terminal one before cyclization and position 5 is the C-terminal one before cyclization), wherein an at least one is an artificial amino acid, and wherein the amino acids are sequentially connected by amide bonds and connected by a head-to-tail cyclization between positions 1 and 5, to form a cyclic penta-peptidomimetic.

According to some embodiments, at least 3 of the amino acids of the peptidomimetic are artificial. According to some embodiments, the at least one artificial amino acid comprises at least one aromatic moiety. According to some embodiments, the at least one artificial amino acid comprises an aromatic side-chain. According to some embodiments, the at least one artificial amino acid comprising at least one aromatic moiety is located in at least one position (from the N-terminus of the pentapeptide before cyclization) selected from the group consisting of position 2, position 4, position 5, or any combination thereof. According to some embodiments, the at least one artificial amino acid comprising the at least one aromatic moiety is located at position 4, position 5, or both.

According to some embodiments, at least one of the 5 amino acids is an alanine derivative. According to some embodiments, the alanine derivative is selected from 1-Nal, 2-Nal, D-Pal(4), 3-(4-Quinolyl)-alanine, and Dip. According to some embodiments, at least one of the 5 amino acids is a phenylalanine derivative. According to some embodiments, the phenylalanine derivative is selected from Phe(3-OMe), Phe(3-OEt), 5-Bromo-2-fluoro-phenylalanine, Phe(2-OCF₃), Phe(2-CF₃), and Phe(2-OMe). According to some embodiments, at least one of the 5 amino acids is a tyrosine derivative. According to some embodiments, the tyrosine derivative is selected from Tyr(3-5-di-Iodo) and Tyr(3-5-di-Bromo). According to some specific embodiments, the peptidomimetic comprises an alanine derivative and a phenylalanine derivative. According to some specific embodiments, the peptidomimetic comprises an alanine derivative and a tyrosine derivative. According to some specific embodiments, the peptidomimetic comprises an alanine derivative, a phenylalanine derivative, and a tyrosine derivative.

According to some embodiments, at least one of the 5 amino acids is a D-amino acid. According to some embodiments, the at least one D-amino acid is located at position 1, position 3, or both.

According to some embodiments, at least one of the 5 amino acids is an aromatic amino acid that is halogenated or haloalkylated. According to some embodiments, the aromatic amino acid is halogenated or haloalkylated with one or more halogens selected from F, Cl, Br, and I. According to some embodiments, the one or more halogens are selected from F, Br, and I. According to some embodiments, the haloalkane is a halomethyl moiety or a halomethoxy moiety. According to further embodiments, the haloalkane is a trifluoromethyl moiety or a trifluoromethoxy moiety. According to some embodiments, the at least one halogenated or haloalkylated amino acid is located at position 2, position 4, position 5, or any combination thereof.

According to some embodiments, the amino acid at position 1 has D-chirality. According to some embodiments, the amino acid at position 1 is selected from the group consisting of D-Ala, D-aThr, D-Pal(4), D-Gln, D-Abu, D-Gly(cPr), D-Dap, and D-Nle(Me). According to some particular embodiments, the amino acid at position 1 is selected from D-Ala and D-aThr.

According to some embodiments, the amino acid at position 2 is an alanine derivative or a phenylalanine derivative. According to some embodiments, the amino acid at position 2 comprises an aromatic moiety. According to some embodiments, the amino acid at position 2 is selected from the group consisting of Pal(3), Phe(3-OEt), Phe(3-OMe), and Phe(3-5-di-F). According to some embodiments, the amino acid at position 2 is Pal(3).

According to some embodiments, the amino acid at position 3 has an anionic residue and/or a cluster of negative atomic partial charges at physiological pH (of about 7.4). According to some embodiments, the amino acid at position 3 is selected from the group consisting of D-Abu(tetrazol-5-yl), D-Cys(-Methyl-2H-tetrazole)), and D-Glu. According to other embodiments, the amino acid at position 3 is selected from D-Abu(tetrazol-5-yl), and D-Cys(-Methyl-2H-tetrazole)).

According to some embodiments, the amino acid at position 4 is a derivative of phenylalanine or tyrosine. According to some embodiments, the derivative of phenylalanine or tyrosine is selected from the group consisting of Tyr(3-5-di-Iodo), Tyr(3-5-di-Bromo), Phe(3-5-di-Bromo), Phe(2-OMe), Phe(3-4-Cl), Phe(2-NO₂), 4-carboxyPhe, Phe(2-OCF₃), Phe(3-OCF₃), Phe(2-CN), Phe(4-guanidino), Phe(F₅), Phe(4-NH₂), Phe(3,4-Cl), Phe(3,4-diOMe), 3-Bromo-5-fluoro-phenylalanine, 5-Bromo-2-fluoro-phenylalanine, Dip, Phe(3-,4-,5-F), and L-thyroxine. According to some embodiments, the amino acid at position 4 is selected from the group consisting of Tyr(3-5-di-Iodo), 5-Bromo-2-fluoro-phenylalanine, Phe(2-OCF₃), Dip, Tyr(3-5-di-Bromo), Phe(3-5-di-Bromo), and Phe(2-OMe). According to further embodiments, the amino acid at position 4 is selected from Tyr(3-5-di-Iodo), 5-Bromo-2-fluoro-phenylalanine, and Phe(2-OCF₃).

According to some embodiments, the amino acid at position 5 comprises an aromatic moiety. According to some embodiments, the amino acid at position 5 is selected from the group consisting of Gly (indan-2-yl), 1-Nal, 2-Nal, 3-(4-Quinolyl))-alanine, L-(7-azaTrp), and Phe(2-CF₃). According to some embodiments, the amino acid at position 5 is Gly (indan-2-yl). According to some further embodiments, the amino acid at position 5 is Phe(2-CF₃).

According to some embodiments, the peptidomimetic is a cyclic pentapeptide wherein:
the amino acid at position 1 is selected from the group consisting of D-Ala, D-aThr, D-Pal(4), D-Gln, D-Abu, D-Gly(cPr), and D-Nle(Me);
the amino acid at position 2 is selected from the group consisting of Pal(3), Phe(3-OEt), Phe(3-OMe), and Phe(3-5-di-F);
the amino acid at position 3 is selected from the group consisting of D-Abu(tetrazol-5-yl), D-Cys(-Methyl-2H-tetrazole)), and D-Glu;
the amino acid at position 4 is selected from the group consisting of Tyr(3-5-di-Iodo), 5-Bromo-2-fluoro-phenylalanine, Phe(2-OCF₃), Dip, Phe(2-OMe), and Phe(3-5-di-Br);
the amino acid at position 5 is selected from the group consisting of L-Gly (indan-2-yl), Phe(2-CF₃), 1-Nal, 3-(4-Quinolyl)-alanine, L-(7-azaTrp), Phe(2-OCF₃), and 2-Nal.

According to some embodiments, the cyclic penta-peptidomimetic is a compound selected from Compound Nos. 1 to 188 as presented in Table 1 and FIG. 1.

According to some embodiments, the cyclic penta-peptidomimetic is selected from Compound Nos. 134, 127, 123, 78, 157, 178, 169, 153, 140, 136, 125, 121, 152, 156, 146, 174, 177, 176, 92, 135, 155, 163, 150, 154, 173, 93, 171, 117, 168, 151, 101, 137, 114, 175, 180, 183, 184, 185, and 30, as presented in Table 1 and FIG. 1.

According to some embodiments, the amino acid at position 3 of the cyclic penta-peptidomimetic is selected from D-Abu(tetrazol-5-yl) and D-Cys(-Methyl-2H-tetrazole) and the cyclic penta-peptidomimetic is selected from Compound Nos. 134, 127, 123, 78, 157, 178, 169, 153, 140, 136, 125, 121, 152, 156, 146, 174, 177, 176, 92, 135, 155, 163, 150, 154, 173, 93, 171, 151, 137, 175, 180, 183, 184, and 185.

According to some embodiments, the cyclic penta-peptidomimetic comprises an amino acid selected from D-Ala, D-aThr, and D-Pal(4) at position 1; the amino acid Pal(3) at position 2; an amino acid selected from D-Abu(tetrazol-5-yl) and D-Glu at position 3; an amino acid selected from Tyr(3-5-di-Iodo), 5-Bromo-2-fluoro-phenylalanine, and Phe(2-OCF₃) at position 4; and an amino acid selected from L-Gly (indan-2-yl), Phe(2-CF₃), 1-Nal, and 3-(4-Quinolyl)-alanine at position 5 and wherein the cyclic penta-peptidomimetic peptidomimetic is selected from Compound Nos. 78, 123, 125, 127, 134, 136, 137, 140, 146, 153, 154, 155, 168, 175, 176, 177, 109, 41, 64, and 47.

According to other embodiments, the amino acid at position 3 is D-Abu(tetrazol-5-yl) and the cyclic penta-peptidomimetic is selected from Compound Nos. 78, 123, 125, 127, 134, 136, 137, 140, 146, 153, 154, 155, 168, 175, 176, and 177.

According to some embodiments, the IL-17 inhibitory activity of the cyclic penta-peptidomimetic, as determined by AlphaLISA, is p(IC₅₀) of 7.0 or above. According to some embodiments, the IL-17 inhibitory activity of the cyclic penta-peptidomimetic, as determined by AlphaLISA, is between 7 and 8, including each value within the specified range.

According to some embodiments, the IL-17 inhibitory activity of the cyclic penta-peptidomimetic, as determined by activation of IL-8, is p(IC₅₀) of 5.5 or above. According to some embodiments, the IL-17 inhibitory activity of the cyclic penta-peptidomimetic, as determined by activation of IL-8, is between 5.5 and 7, including each value within the specified range.

According to some embodiments, the cyclic penta-peptidomimetic inhibits the interaction between an IL-17A homodimer and an IL-17R.

According to some embodiments, the cyclic penta-peptidomimetic inhibits the interaction between an IL-17F homodimer and an IL-17R.

According to some embodiments, the cyclic penta-peptidomimetic inhibits the interaction between an IL-17AF heterodimer and an IL-17R.

According to an aspect of the present invention, there is provided a pharmaceutical composition, comprising at least one cyclic penta-peptidomimetic as disclosed herein above, and at least one excipient, salt, or carrier.

According to some embodiments, the pharmaceutical composition comprises at least two different cyclic penta-peptidomimetics.

According to some embodiments, the pharmaceutical composition is for the treatment of an IL-17 associated disease, condition, or disorder.

According to some embodiments, the IL-17 associated disease, condition, or disorder, is selected from the group consisting of psoriasis, psoriatic arthritis, rheumatoid arthritis, spondyloarthritis, multiple sclerosis, axial spondyloarthritis, ankylosing spondylitis, hidradenitis suppurativa, systemic lupus erythematosus, palmoplantar pustulosis (PPP), atopic dermatitis, asthma, inflammatory bowel disease, chronic obstructive pulmonary disease (COPD), age-associated skin condition, and allograft rejection.

According to some embodiments, the psoriasis is plaque psoriasis.

Any stage and severity of plaque psoriasis may be treated with the compositions of the present invention, including mild, moderate, and severe stages. According to some embodiments, the disease is moderate to severe plaque psoriasis. According to some embodiments, the disease is moderate plaque psoriasis. According to other embodiments, the disease is severe plaque psoriasis.

According to some embodiments, the pharmaceutical composition is formulated for local, parenteral or enteral administration. According to some embodiments, the pharmaceutical composition is formulated for topical administration, intravenous administration, or oral administration. According to some embodiments, the pharmaceutical composition is formulated for administration by injection, infusion, inhalation or as a nasal spray. According to yet other embodiments, the pharmaceutical composition is formulated for intradermal or subcutaneous administration. According to particular embodiments, the pharmaceutical composition is formulated for administration locally to the dermis. According to other embodiments, the composition is formulated for administration by electroporation, using methods and devices known in the art.

According to other embodiments, a cosmetic composition is provided, comprising at least one cyclic penta-peptidomimetic as disclosed herein above, and at least one cosmetically acceptable excipient, salt, or carrier.

According to some embodiments, the cosmetic composition is formulated for local administration. According to some embodiments, the local administration is topical administration.

According to some embodiments, the cosmetic composition is formulated as a lotion, a cream, an ointment, a paste, a gel, a hydrogel and the like, or as a powder.

According to other embodiments, the cosmetic composition is formulated as a solution, a mist, or a spray.

According to some embodiments, the cosmetic composition is for use in the prevention, treatment, alleviation of a cosmetic condition, disorder or disease.

According to some embodiments, the cosmetic condition comprises a skin disorder or an appearance of the skin.

According to some embodiments, the cosmetic condition is selected from, but not limited to: acne (particularly mild to moderate, and including post-acne marks), excess oil production (including seborrhoea), enlarged pores, hyperpigmentation (including post-inflammatory hyperpigmentation), melasma, dark spots, sunspots and age spots, age signs and wrinkles (including fine lines and deep wrinkles), loss of skin elasticity, sagging and dull skin, sensitive and reactive skin (e.g., rosacea, or eczema), skin redness and irritation, rosacea-prone skin, eczema and atopic dermatitis marks, barrier dysfunction, dry and dehydrated skin, flaky, rough skin, tightness and discomfort skin, xerosis (severe dry skin), eye dark circles and puffy eyes, under-eye pigmentation, puffiness and swelling, fine lines and crepey skin.

According to an aspect of the present invention, there is provided a method for treating a patient suffering from an IL-17 associated disease, condition, or disorder, comprising the step of administering to a patient in need thereof an effective amount of at least one cyclic penta-peptidomimetic as disclosed herein above, or a pharmaceutically acceptable salt thereof.

According to some embodiments, the step of administering to the patient in need thereof an effective amount of the at least one cyclic penta-peptidomimetic, is performed following receiving an affirmative result in a preliminary step, the preliminary step comprising determining whether the patient has an elevated level of at least one IL-17-induced chemokine or effector. According to some embodiments, the at least one IL-17-induced chemokine or effector is selected from the group consisting of IL-8, Gro-alpha, IL-1A, TNF-alpha, IL-6, IL-22, S100a7a, CK-16, and IL-1beta.

According to some embodiments, administering a cyclic penta-peptidomimetic according to the present invention is part of a treatment regimen comprising administering at least one additional therapeutic agent. According to some embodiments, the additional therapeutic agent is an anti-inflammatory agent or an immunosuppressant.

According to some embodiments, the IL-17 associated disease, condition, or disorder, is selected from the group consisting of psoriasis, psoriatic arthritis, rheumatoid arthritis, spondyloarthritis, multiple sclerosis, axial spondyloarthritis, ankylosing spondylitis, hidradenitis suppurativa, systemic lupus erythematosus, palmoplantar pustulosis (PPP), atopic dermatitis, asthma, inflammatory bowel disease, COPD, age-associated skin condition, and allograft rejection.

According to other embodiments, a cosmetic composition is provided, for use in the prevention, treatment, alleviation of a cosmetic condition, disorder or disease.

According to some embodiments, the present invention provides a method for preventing, treating, or alleviating a cosmetic condition or disorder, the method comprising administering an effective amount of a compound according to the present invention, or a cosmetically acceptable salt thereof.

According to some embodiments, the cosmetic condition comprises a skin disorder or an appearance of the skin.

According to some embodiments, the cosmetic condition is selected from, but not limited to: acne (particularly mild to moderate, and including post-acne marks), excess oil production (including seborrhoea), enlarged pores, hyperpigmentation (including post-inflammatory hyperpigmentation), melasma, dark spots, sunspots and age spots, age signs and wrinkles (including fine lines and deep wrinkles), loss of skin elasticity, sagging and dull skin, sensitive and reactive skin (e.g., rosacea, or eczema), skin redness and irritation, rosacea-prone skin, eczema and atopic dermatitis marks, barrier dysfunction, dry and dehydrated skin, flaky, rough skin, tightness and discomfort skin, xerosis (severe dry skin), eye dark circles and puffy eyes, under-eye pigmentation, puffiness and swelling, fine lines and crepey skin.

According to some embodiments, the cosmetic composition is administered by local administration. According to some embodiments, the local administration is topical administration. According to some embodiments, the local administration is intradermal administration.

According to some embodiments, the cosmetic composition is administered as a lotion, a cream, an ointment, a paste, a gel, a hydrogel and the like, or as a powder.

According to other embodiments, the cosmetic composition is administered as a solution, a mist, or a spray.

According to some embodiments, the present invention provides a dosage form suitable for administration wherein the administration route is selected from injection, infusion, inhalation or as a nasal spray and wherein the dosage form comprises at least one container which contains the pharmaceutical composition according as disclosed herein.

The present invention also provides, according to yet another aspect, a kit comprising at least one container containing the pharmaceutical composition as disclosed herein and instructions for using the kit.

It is to be understood that any combination of each of the aspects and the embodiments disclosed herein is explicitly encompassed within the disclosure of the present invention.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE FIGURES

The novel features described herein are set forth with particularity in the appended claims. A better understanding of the characteristics and advantages of the features described herein will be obtained by reference to the following detailed description that sets forth illustrative examples, in which the principles of the features described herein are utilized, and the accompanying drawings of which:

FIG. 1 depicts atomic structures of some of the peptidomimetics of the invention.

FIGS. 2A-2B are exemplary bar charts showing results of assays performed with eight different peptidomimetics of the present invention (Compound Nos. 24, 30, 35, 37, 51, 57, 67, and 71, with each bar chart representing a separate peptidomimetic), testing their IL-17A-IL-17R inhibitory activity. The IL-17A-IL-17R binding signal without any peptidomimetics was set as the baseline level (i.e., 100%). FIG. 2A shows the results of AlphaLISA assay, the signal being indicative of IL-17A-IL-17R binding, as a function of the concentration of the exemplary peptidomimetic. FIG. 2B shows the results of indirect competitive ELISA signal, indicative of IL-17A-IL-17R binding, as a function of the concentration of the peptidomimetics, for the exemplary peptidomimetics.

FIGS. 3A-3B are exemplary bar charts showing results of AlphaLISA performed on four exemplary peptidomimetics of the present invention (Compound Nos 24, 30, 51 and 71, with each bar chart representing a separate peptidomimetic), testing the inhibitory activity thereof on the interaction of each of IL-17F and IL-17AF with IL-17R. The IL-17-IL-17R binding signal without any peptidomimetics was set as the baseline level (i.e., 100%). FIG. 3A shows the AlphaLISA signal indicative of IL-17F-IL-17R binding, as a function of the concentration of the peptidomimetic. FIG. 3B shows the AlphaLISA signal indicative of IL-17AF-binding to IL-17R, as a function of the concentration of the peptidomimetic.

FIGS. 4A-4B are graphs representing the inhibitory effect of four different peptidomimetics (Compound Nos. 30, 51, 67, and 71) of the present invention on the IL-17-IL-17R interaction in live cells. FIG. 4A shows the IL-8 level (as an IL-17 activation indicator) in the cell culture medium as a function of the concentration of the peptidomimetics added thereto.

FIG. 4B is a bar-chart comparing the p(IC₅₀) values of 18 different peptidomimetics which were added to the cell culture medium, as measured and calculated according to AlphaLISA (black bar), ELISA (white bar), and cell culture (grey bar).

FIGS. 5A-5B are representations of the effect of treatment with the peptidomimetic designated Compound No. 30, on the skin pathology of psoriasis model mice (induced by Imiquimod, IMQ). FIG. 5A shows photographs, and corresponding histological images, of the skin of psoriasis-induced mice treated with the peptidomimetic (“IMQ+ID−30), treated with an approved steroid drug (0.5 mg/g betamethasone, “IMQ+steroid”, positive control), and non-treated (“IMQ+vehicle”, negative control), as well as non-psoriasis-induced mice (“no-IMQ”), respectively. FIG. 5B is a bar-chart showing and comparing the average measured skin thickness of each mouse group as described in FIG. 5A.

FIGS. 6A-6H are bar-charts showing and comparing the average measured expression of genes (in mRNA levels determined using quantitative real-time PCR) related to IL-17 activation in each mouse group as described in FIG. 5A. FIG. 6A—Cxcl1; FIG. 6B—IL-1A; FIG. 6C—TNF-alpha (TNFa); FIG. 6D—IL-6; FIG. 6E—IL-17A; FIG. 6F—IL-22; FIG. 6G—S100a7a; and FIG. 6H—CK-16.

FIG. 7 is a bar-chart showing and comparing the average measured Cxcl1 level in mice serum of each mouse group as described in FIG. 5A.

FIGS. 8A and 8B are two bar charts showing results of AlphaLisa assays performed for comparing Compound No. 171 (FIG. 8A) and a similar compound in which D-Abu(tetrazol-5-yl) at position 3 was exchanged for D-Histidine (FIG. 8B). The AlphaLisa assay was performed to test the inhibitory effect of the two peptidomimetics on the interaction between IL-17A and IL-17R. The binding signal of IL-17A to IL-17R without any peptidomimetics was set as the baseline level (i.e., 100%).

FIGS. 9A and 9B are exemplary charts showing results of assays performed with eight different cyclic penta-peptidomimetics of the present invention (Compound Nos. 78, 150, 134, and 127, with each chart representing a separate peptidomimetic), testing their IL-17A-IL-17R inhibitory activity. FIG. 9A shows the results of AlphaLISA assay, the signal being indicative of IL-17A-IL-17R binding, as a function of the concentration of the exemplary peptidomimetic. The IL-17A-IL-17R binding signal without any peptidomimetics was set as the baseline level (i.e., 100%). FIG. 9B shows the IL-8 level (as an IL-17 activation indicator) in the cell culture medium as a function of the concentration of the peptidomimetics added thereto.

### DETAILED DESCRIPTION OF THE INVENTION

All terms used herein are as commonly used in the art, unless defined otherwise herein. In case of contradiction between the common definition and the definition of the present application, the definition herein is decisive.

As used herein, the term “IL-17”, unless expressly stated otherwise, refers to an IL-17A homodimer (“IL-17A”), an IL-17F homodimer (“IL-17F”), and/or an IL-17AF heterodimer (“IL-17AF”). IL-17A is a member of the IL-17 family, and is a homodimeric glycoprotein with a molecular mass of 35 kDa. Each subunit of the IL-17A homodimer, named “chain A” and “chain B”, is composed of a pair of anti-parallel β-sheets. In the IL-17AF heterodimer, the IL-17A subunit is referred to as “chain A” while the IL-17F subunit is referred to as “chain F”. As used herein, the term “IL-17R” refers to the heterodimer of IL-17 receptor A (“IL-17RA”) and IL-17 receptor C (“IL-17RC”), unless expressly stated otherwise. Thus, the term “IL-17-IL-17R interaction” refers to an interaction of at least one of IL-17A, IL-17F, and IL-17AF with IL-17R. This interaction is considered to be responsible for the biological activity of the cytokine.

As used herein, the term “amino acid” (“AA”) refers to any compound which contains both an amino (—NH₂) and a carboxylic acid (—COOH) functional group. The term “amino acid” also encompasses amino acid residues, which are amino acids in which at least one of the amino and carboxylic functional groups is linked to a neighboring compound by an amide bond.

As used herein, the term “DNA-encoded amino acid” refers to an amino acid which is one of the known 20 L-amino acids which are encoded for by nucleic acid sequences and are utilized by a ribosome in the translation process of protein synthesis. DNA-encoded amino acids also include L-chirality stereoisomers of the known 20 naturally-occurring amino acids that are utilized by a ribosome. Two additional amino acids, Selenocysteine and Pyrrolysine, that are in some species coded for by codons that are usually interpreted as stop codons, are likewise utilized by the ribosome.

Accordingly, the terms “non-DNA-encoded amino acid” or “artificial amino acid” refers to an amino acid which is not a DNA-encoded amino acid. Artificial amino acids also include D-chirality configurations (i.e., isomer or D-amino acids) of DNA-encoded amino acids. The terms “unnatural amino acid” and “non-proteinogenic amino acid” may be used interchangeably with the terms “non-DNA-encoded amino acid” or “artificial amino acid”. In addition, the term “non-proteinogenic amino acid” refers to an amino acid that is not naturally encoded or found in the genetic code, and is not incorporated biosynthetically into proteins during translation. Non-proteinogenic amino acids may be “unnatural amino acids” (amino acids that do not occur in nature) or “naturally-occurring non-proteinogenic amino acids” (e.g., norvaline, ornithine, homocysteine, etc.).

As used herein, the term “known L- or D-amino acids” refers to the 19 known amino acids in either L-configuration or D-configuration, as well as the achiral amino acid glycine.

As used herein, the term “cyclic pentapeptide” may be used interchangeably with “cyclic penta-peptidomimetic”. The term “cyclic penta-peptidomimetic” may be referred to “macrocycle” or “compound” throughout the specification as disclosed herein.

As used herein, the term “substituent” refers to a first atom or functional group that has replaced a second atom or functional group of an amino acid, wherein the second atom or functional group may be a hydrogen atom or any other molecule, compound or moiety. Suitable substituents include, without limitation, halo, hydroxy, mercapto, oxo, nitro, halogen, haloalkyl, alkyl, alkaryl, aryl, pyridyl, cyclo-propyl, cyclo-butyl, cyclohexyl, aralkyl, alkoxy, thioalkoxy, aryloxy, amino, cyano, guanidino, alkoxycarbonyl, amido, carboxy, methyl, methoxy, alkanesulfonyl, alkylcarbonyl, thienyl, thiazolyl, furyl, naphthyl, and indanyl functional groups, or a combination thereof.

As used herein, the term “modified amino acid” refers to a known L- or D-amino acid which includes at least one substituent, as defined above. Similarly, the term “amino acid derivative”, e.g., “phenylalanine derivative”, and the like, refers to a particular amino acid from the 20 known amino acids, with L or D chirality, which includes at least one substituent as described above.

As used herein, the term “cyclic group” refers to a carbon-containing ring made up of at least one of a 3-membered cyclic group, a 4-membered cyclic group, a 5-membered cyclic group, and a 6-membered cyclic group. Aromatic, non-aromatic, and heterocyclic rings are included within the scope of the invention. Higher membered cyclic groups are also encompassed in the term “cyclic group”. The term “cyclic conformation” may be used interchangeably with the term “cyclic group”.

As used herein, the term “peptide bond” refer to an amide bond between the carboxyl moiety of an amino acid and the amino moiety of the next amino acid, within a peptide or peptidomimetic amino acid chain.

The position numbers of the amino acids, i.e., position 1, position 2, etc., as used herein, are significant to establish the sequential order of the amino acids in the peptide. In linear peptides, numbering is typically started from the reside at the N-terminus, referred to herein as “position 1”, “first amino acid”, and the like and the position number of each amino acid in the peptide chain is determined according to the number of amino acids from the start of the chain. For the sake of illustration, the first amino acid is at position 1, the following amino acid which shares a peptide bond (i.e., amide bond) with the first amino acid is at position 2, and so on. As used herein, the terms “end of peptide”, “last/end amino acid”, and the like, refer to the amino acid which is located at the C-terminus end of the amino acid chain. The position of the last amino acid depends on the number of amino acids in the peptide/peptidomimetic. As an illustrative example, when the peptidomimetic consists of 5 AA, the last amino acid is defined as being at position 5. However, when the peptide contains a head-to-tail cyclization, the terms of “beginning” and “end” of the peptide (i.e., N-terminus and C-terminus) become irrelevant, as the peptide forms a continuous cycle. In such situations, and as in the present invention, the AA in the last position, for example position 5, is between the AA at position 4 and the AA at position 1. Typically, in solid phase peptide synthesis, the peptide is synthesized from the C-terminus to the N-terminus, namely, the last amino acid residue (position 5 of the present invention) is attached first to the solid phase, and the other residues are built on the N-terminus of this residue through its N-terminus. Other synthetic schemes may also apply.

As used herein, the terms “increase” and “decrease” mean, respectively, to cause a statistically significantly (i.e., p<0.1) increase or decrease of at least 5%.

As used herein, the recitation of a numerical range for a variable is intended to convey that the variable is equal to any of the values within that range. Thus, for a variable which is inherently discrete, the variable is equal to any integer value within the numerical range, including the end-points of the range. Similarly, for a variable which is inherently continuous, the variable is equal to any real value within the numerical range, including the end-points of the range. As an example, and without limitation, a variable which is described as having values between 0 and 2 takes the values 0, 1 or 2 if the variable is inherently discrete, and takes the values 0.0, 0.1, 0.01, 0.001, or any other real values >0 and <2 if the variable is inherently continuous.

As used herein, unless specifically indicated otherwise, the word “or” is used in the inclusive sense of “and/or” and not the exclusive sense of “either/or.”

### Amino Acids (AAs)

According to some embodiments, the artificial amino acids of the present invention comprise modified DNA-encoded AAs, D-chirality configurations of DNA-encoded amino acids, and/or modifications of the D-chirality configurations. According to some embodiments, the DNA-encoded AAs, in modified form, from which the peptidomimetics according to the present invention are comprised, are selected from glycine, alanine, phenylalanine, tyrosine, leucine, norleucine, aspartate, tryptophan, and cysteine. According to some embodiments, the D-chirality configurations of DNA-encoded amino acids, from which the peptidomimetics of the present invention are comprised, are selected from D-Alanine, D-Allothreonine, D-Norleucine, D-Serine, D-Glutamine, and D-Glutamic acid. According to some embodiments, the peptidomimetics according to the present invention comprises at least one amino acid selected from D-Norleucine, D-Alanine, D-Phenylalanine, D-Aspartate, and D-Cysteine.

According to some embodiments, the modification of the DNA-encoded AA or D-chirality configuration thereof, includes substitution of at least one hydrogen moiety/atom and/or at least one carbon moiety/atom with a moiety selected from the group consisting of: hydroxy, methyl, trifluoromethyl, methoxy, trifluoromethoxy, ethoxy, carboxyl, cyano, nitro, halogen, guanidino, pyridyl, thienyl, tetrazole, thiazolyl, furyl, cyclo-propyl, cyclo-butyl, cyclohexyl, naphthyl, and indanyl moieties, a derivative thereof or a combination thereof. According to some particular embodiments, the modification includes substitution of at least one hydrogen moiety/atom and/or at least one carbon moiety/atom with a moiety comprising at least one cyclic group. According to some embodiments, the moiety comprising at least one cyclic group is selected from pyridyl, thienyl, thiazolyl, furyl, cyclo-butyl, cyclohexyl, naphthyl, and indanyl.

According to some particular embodiments, the artificial amino acids are selected from the group consisting of: D-Alanine (D-Ala), D-Allothreonine (D-aThr), Allothreonine (aThr), D-Diaminopimelic (D-Dap), D-Norleucine (D-Nle), R-2-aminohepatonic acid (D-Nle(Me)), D-Cyclopropylglycine (D-Gly(cPr)), D-4-pyridyl-alanine (D-Pal(4)), Cyclopropylglycine (Gly(cPr)), D-Glutamine (D-Gln), D-Serine (D-Ser), D-homophenylalanine (D-hPhe), D-2-alpha-Aminobutyric-acid (D-Abu), L-2-pyridyl-alanine (Pal(2)), L-3-pyridyl-alanine (Pal(3)), Beta-(2-thienyl)-Ala (2Thi), 1-Aminocyclopropane-1-carboxylic acid (Ac3c), Beta-Cyclobutyl-L-Alanine (Ala(cBu)), Cyclohexylalanine (Cha), 6-hydroxynorleucine (L-Nle(6-OH)), 3-fluoro-phenylalanine (Phe(3-F)), 3-methyl-phenylalanine (Phe(3-Me)), 3-hydroxy-phenylalanine (Phe(3-OH)), 3-methoxy-phenylalanine (Phe(3-OMe)), 3-ethoxy-phenylalanine (Phe(3-OEt)), 2-methoxy-phenylalanine (Phe(2-OMe)), Beta-(4-thiazolyl)-Ala (Tza), Homoleucine (hLeu), L-3-thienylalanine (3Thi), D-2-Furylalanine (beta-(2-furyl)-D-Ala), L-2-Furylalanine (beta-(2-furyl)-Ala), 3-5-difluoro-phenylalanine (Phe(3-5-di-F)), D-Glutamic acid (D-Glu), gamma-Carboxy-D-glutamic-acid (D-Gla), D-2-alpha-Aminobutyric-acid (tetrazol-5-yl) (D-Abu(tetrazol-5-yl)), D-Cys(-Methyl-2H-tetrazole), D-2-aminoadipic acid (D-Aad), D-Asp-[1-nipecotic acid] (D-Asp-[-1Nip]), D-Cysteic Acid (D-Cys (O₃H)), 3,5-Di-Iodo-tyrosine (Tyr(3-5-di-Iodo)), (2-naphthyl)-alanine (2-Nal), (1-naphthyl)-alanine (1-Nal), 4-carboxyphenylalanine (4-carboxyPhe), 5-methoxy-tryptophan, L-9-Anthrylalanine (Ala (9-Anth)), 3-3-diphenyl-L-alanine (Dip), 3-4-dimethoxy-phenylalanine (Phe(3-4-diOMe)), 3,5-dichloro-tyrosine (Tyr(3-5-di-Cl), L-isoAsp-[3-Aminobenzoic acid] (L-isoAsp-[-3Abz]), 2-(trifluoromethoxy)-phenylalanine (Phe(2-OCF₃)), L-2-Cyanophenylalanine (Phe(2-CN)), 2-nitro-phenylalanine (Phe(2-NO₂)), 4-Guanidinophenylalanine (Phe(4-guanidino)), 3-4-dichloro-phenylalanine (Phe(3-4-Cl)), (S)-2-Methoxy-phenylglycine (Phg(2-OMe)), 4-Amino-phenylalanine (Phe(4-NH₂)), 3-iodo-tyrosine (Tyr(3-Iodo)), 5-Bromo-2-fluoro-phenylalanine, pentafluoro-phenylalanine (Phe(F₅)), 7-Azatryptophan (L-(7-azaTrp)), 3-5-di-Bromo-Tyrosine (Tyr(3-5-di-Bromo)), 2-trifluoromethyl-phenylalanine (Phe(2-CF₃)), 3-(4-Quinolyl)-alanine, 3-Nitro-tyrosine (Tyr(3-NO₂)), 3-Bromo-5-fluoro-phenylalanine, L-2-indanylglycine (Gly (indan-2-yl)), iso-aminoadipic acid-[-Val-ol] (isoAad-[-Val-ol]), iso-aminoadipic acid-[1-nipecotic acid-3-Aminobenzoic acid] (isoAad-[-1Nip-3 Abz]), L-Asp-[-GLY-GLY-GLY-3-Aminobenzoic acid (L-Asp-[-GLY-GLY-GLY-3.Abz]), N-(2-4-dimethoxybenzyl)-Gly) (Dmb (Gly)), 5-phenyl-L-norvaline (Nva(Ph)), Homoserine (Hse), L-thyroxine, 2-Amino-3-(7-methoxy-4-coumaryl) propionic acid (beta-(7-methoxy-coumarin-4-yl)-Ala), 3,4,5-Trifluorophenylalanine (Phe(3,4,5-F)), 4-guanidino-phenylalanine (Phe(4-guanidino)), 3-methyl-tyrosine (Tyr(3-Me)), 3,5-Di-Bromophenylalanine (Phe(3-5-di-Br)), N-methylated 3-iodo-tyrosine (Nme-Tyr(3-Iodo)), Kynurenine (Asp(Ph(2-NH₂))), 2-amino-2-indancarboxylic-acid (Aic), S-xanthyl-cysteine (Cys(Xan)), Cyclopropylalanine (Ala (cPr)), Sarcosine (Nme-Gly), 4-phenyl-L-phenylalanine (Bip), 4-Benzoylphenylalanine (Bpa), 1-Benzyl-D-histidine (D-His(1-Bn)), Homotyrosine (hTyr), D-Serine-acetate (D-Ser (Ac)), 3,5-dimethyl-tyrosine (Tyr(3-5-di-Me)), L-isoGlu-[-3-iodotyrosine-D-homophenylalanine-methylserine-D-citrulline (L-isoGlu-[-Tyr(3-I)-DhPhe-Ser (Me)-DCit]), L-2-Amino-3-guanidinopropionic-acid (Ala (guanidino)), 2-Amino-3,3-bis(4-fluorophenyl) propanoic acid, 3-Nitro-D-phenylalanine (D-Phe(3-NO₂)), and beta-hydroxyphenylalanine (Phe (bR-OH)).

Some of the amino acids used in this invention are those which are available commercially or are available by routine synthetic methods. Certain amino acids may require special methods for preparation and/or for incorporation into the peptidomimetic, and either sequential, divergent or convergent synthetic approaches are useful in this invention. Natural coded amino acids and their derivatives are represented by three-letter codes according to IUPAC conventions. When there is no indication, the L isomer was used. The D isomers are indicated by “D” or “(D)” before the residue abbreviation or by using the lower case of the amino acid code.

The peptides of the present invention may be produced by any synthetic method known in the art. Synthetic methods include exclusive solid phase synthesis, partial solid phase synthesis, fragment condensation, or classical solution synthesis. In some embodiments, synthetic peptides are purified by preparative high-performance liquid chromatography, or by other methods known in the art.

In solid phase peptide synthesis, the peptide is synthesized from the C-terminus to the N-terminus, namely, the last amino acid residue is attached first to the solid phase, and the other residues are built on the N-terminus of this residue through its N-terminus, and relies on the orthogonal protection of reactive groups but this may vary in other synthesis methods. According to some embodiments, the peptidomimetic is synthesized according to the order of the AA from position 5 to position 1, i.e., from the C-terminus to the N-terminus of the peptide. According to some further embodiments, the peptidomimetic, particularly a peptidomimetic which eventually forms a head-to-tail cyclization, is synthesized starting with at least one AA at any position (e.g., position 2, position 1, etc.) so long as the sequential order of the amino acids is maintained.

Conjugates of the peptidomimetics of the present invention, and an additional molecule, are also included within its scope, provided that the conjugate has similar or even improved properties compared to the parenteral compound. According to some embodiments, the conjugate comprises a cyclic penta-peptidomimetic described above and a carrier molecule. A conjugate according to the present invention comprises any cyclic penta-peptidomimetic according to the present invention, conjugated to a carrier molecule, protein or another moiety which improves the cyclic penta-peptidomimetic's solubility, stability or permeability.

According to some embodiments, the conjugate comprises at least one non-proteinaceous moiety.

The carrier or other moiety may be connected, according to some embodiments, to a side chain of any amino acid of positions 1-5 of the cyclic penta-peptidomimetic, directly or through a spacer or linker. Each possibility represents a separate embodiment.

According to some embodiments, the conjugate comprises one or more permeability-enhancing moieties. Any moiety known in the art to facilitate actively or passively or enhance permeability of the compound into cells or through biological membranes may be used.

According to some embodiments, the conjugate comprises one or more stabilizing moieties.

“Permeability” refers to the ability of an agent or substance to penetrate, pervade, or diffuse through a barrier, membrane, or a skin layer. A “cell permeability” or a “cell-penetration” moiety refers to any molecule known in the art which is able to facilitate or enhance penetration of molecules through membranes. Non-limitative examples of a permeability-enhancing moiety include: hydrophobic moieties such as fatty acids, steroids and bulky aromatic or aliphatic compounds; moieties which may have cell-membrane receptors or carriers, such as steroids, vitamins and sugars, natural and non-natural amino acids and transporter peptides. According to some embodiments, the hydrophobic moiety is a lipid moiety or an amino acid moiety.

Water soluble, synthetic polymers, particularly polyalkylene glycols (PEGs), are widely used to conjugate therapeutically active molecules. These therapeutic conjugates have been shown to alter pharmacokinetics favorably by prolonging circulation time and decreasing clearance rates, decreasing systemic toxicity, and in several cases, displaying increased clinical efficacy. The process of covalently conjugating polyethylene glycol, PEG, to therapeutically active molecules is commonly known as “PEGylation”. Where appropriate, the abbreviation PEG is used in combination with a numeric suffix, which indicates the number of repeating ethylene glycol units.

According to some embodiments, the cyclic penta-peptidomimetic of the present invention is PEGylated. According to some embodiments, a PEG chain is connected, directly or through a spacer or linker, to any of positions 1-5 of the cyclic penta-peptidomimetic. Each possibility represents a separate embodiment. In some embodiments, the length of the PEG chain is between 8-20 units, including each value within the specified range. In some embodiments, the length of the PEG chain is between 8-16 units, including each value within the specified range. In some embodiments, the length of the PEG chain is between 10-14 units, including each value within the specified range.

### Peptidomimetic Synthesis

Solid phase peptide synthesis procedures are well known in the art and further described in “Solid-Phase Synthesis: A Practical Guide”, Ed. Steven A. Kates and Fernando Albericio, CRC Press; 1st Edition (2000). A skilled artesian may synthesize any of the cyclic penta-peptidomimetics of the present invention using any method known in art. For example, the cyclic penta-peptidomimetics may be synthesized using an automated peptide synthesizer using standard chemistry such as, for example, t-Boc or Fmoc chemistry. The methods include exclusive solid phase synthesis, partial solid phase synthesis, fragment condensation, and classical solution synthesis.

Coupling of the amino acids in solid phase peptide chemistry can be achieved by means of a coupling agent such as but not limited to dicyclohexycarbodiimide (DCC), bis(2-oxo-3-phosphinic chloride (BOP-C1), benzotriazolyl-N-oxytrisdimethyl-oxazolidinyl) aminophosphonium hexafluoro phosphate (BOP), 1-oxo-1-chlorophospholane (Cpt-C1), hydroxybenzotriazole (HOBT), or mixtures thereof.

The use of additional coupling reagents including, but not limited to: coupling reagents such as PyBOP (Benzotriazole-1-yl-oxy-tris-pyrrolidino-phosphonium hexafluorophosphate), PyBrOP (Bromo-tris-pyrrolidino-phosphonium hexafluoro-phosphate), HBTU (2-(1H-Benzotriazole-1-yl)-1,1,3,3-tetramethyluronium hexafluoro-phosphate), TBTU (2-(1H-Benzotriazole-1-yl)-1,1,3,3-tetramethyluronium tetrafluoroborate), may be also utilized for synthesizing the peptide compounds of the present invention.

Additional coupling chemistries may be used, such as pre-formed urethane-protected N-carboxy anhydrides (UNCAs), pre-formed acyl halides, and most preferably, acyl chlorides.

Such coupling may take place at room temperature and also at elevated temperatures, in solvents such as toluene, DCM (dichloromethane), DMF (dimethylformamide), DMA (dimethylacetamide), NMP (N-methyl pyrrolidinone), dioxane, tetrahydrofuran, diglyme and 1,3 dichloropropane, or mixtures of the above.

Synthesized cyclic penta-peptidomimetics of the present invention can be purified by preparative high-performance liquid chromatography.

The term “solid phase” as used herein is intended to mean an insoluble polymeric matrix whereupon a molecule, e.g., a cyclic penta-peptidomimetic, can be synthesized or coupled with or without a linker or spacer in-between. Solid support resins are typically used in peptide synthesis. These polymers are generally employed in the form of beads. Polymer resins preferred for peptide synthesis are polystyrenes, polyacrylamides and the like, specifically copolymers of styrene and divinylbenzene. Prior to the coupling with the first amino acid, the solid support resin contains surface functionality or can be derivatized to contain surface functionality which can interact with an amine group of an amino acid (or peptide) so as to attach the amino acid (or peptide) to the support directly or indirectly through the amine group of the peptide. The term “solid phase resin” as used herein is not limited to the parent commercial derivatized resins, in their form prior the first coupling of amino acid or peptide. Rather, after the first coupling of amino acid and during the peptide synthesis, while the resin is coupled to a growing peptide, the resin is still considered a solid phase resin.

### Peptidomimetics

As used herein, the term “peptidomimetic” refers to a chain of AAs that contains at least one artificial amino acid and/or at least one bond that is not a peptide bond. According to some embodiments, the peptidomimetic comprises a chain of AAs linked by peptide bonds, in which at least 20% of the AAs in the chain (e.g., at least one AA of the pentapeptide) are artificial amino acids.

According to some embodiments, the peptidomimetic comprises at least 20% amino acids which are modifications of the known L or D amino acids. According to some embodiments, the peptidomimetic comprises 30% artificial amino acids, 40%, 50%, 60%, 70%, 80%, 90% artificial amino acids. Each possibility represents a separate embodiment of the invention.

According to some embodiments, the peptidomimetic or a conjugate comprising the peptidomimetic, consists of up to 20 AAs. According to some further embodiments, the peptidomimetic or conjugate consists of up to 15 AAs, up to 12 AAs, up to 10 AAs, up to 9 AAs, up to 8 AAs, up to 7 AAs, up to 6 AAs, or up to 5 AAs. Each possibility represents a separate embodiment of the invention. According to some embodiments, the peptidomimetic or conjugate consists of between 5 and 10 AAs, including each value therebetween. According to some embodiments, the peptidomimetic or conjugate consists of between 3 and 12 AAs, between 3-10 AAs, between 3-7 AAs, between 3-5 AAs, between 4 and 10 AAs, between 4 and 8 AAs, between 4 and 6 AAs, between 5 and 12 AAs, between 5 and 8 AAs, or between 5 and 7 AAs. Each possibility represents a separate embodiment of the invention.

According to some specific embodiments, the peptidomimetic consists of 5 AAs, referred to herein also as “pentapeptide”, “cyclic pentapeptide”, or “cyclic penta-peptidomimetic”. According to particular embodiments, the pentapeptide comprises 1, 2, 3, 4 or 5 artificial amino acids.

For further clarification, and as explained above, a peptidomimetic consisting of 5 AAs includes 4 peptide bonds, i.e., a peptide bond between the AA at position 1 and the AA at position 2, a peptide bond between the AA at position 2 and the AA at position 3, etc. In the embodiments of the peptidomimetic having head-to-tail (C-terminus to N-terminus) cyclization, the peptide has an additional peptide bond between the AA at the 5th position (C-terminus) and the AA at position 1 (N-terminus), i.e., overall, 5 peptide bonds.

According to some embodiments, the peptidomimetic includes at least one non-amide bond. According to some embodiments, the peptidomimetic includes at least one additional AA, which is bonded to one of the AAs of the peptidomimetic via a non-peptide bond. Further embodiments of the at least one additional AA are set out herein in the context at position 5 of the peptidomimetic.

According to some embodiments, the peptidomimetic comprises 1 artificial AA. According to some embodiments, the peptidomimetic comprises 2 artificial AAs. According to some embodiments, the peptidomimetic comprises 3 artificial AAs. According to some embodiments, the peptidomimetic comprises 4 artificial AAs. According to some embodiments, the peptidomimetic comprises 5 artificial AAs. According to some embodiments, the peptidomimetic consists of 5 AAs, of which 2 AAs are artificial. According to some further embodiments, the peptidomimetic consists of 5 AAs, of which 3 AAs, 4 AAs, or 5AAs are artificial. Each possibility represents a separate embodiment of the invention. According to some particular embodiments, the peptidomimetic consists of only artificial AAs.

According to some embodiments, the peptidomimetics comprise at least one AA having at least one cyclic group. According to some embodiments, the peptidomimetics comprise at least 2, at least 3, or at least 4 AAs having at least one cyclic group. According to some embodiments, at least one of the cyclic groups is aromatic. According to some embodiments, at least one of the cyclic groups is heterocyclic. According to some embodiments, at least one of the cyclic groups is a 3 to 6-membered cyclic group. According to some embodiments, the peptidomimetic consists of 5 AAs, of which one AA has a cyclic group. According to some further embodiments, the peptidomimetic consists of 5 AAs, of which 2, 3, or 4 AAs have a cyclic group. Each possibility represents a separate embodiment of the invention.

According to some embodiments, the at least one AA comprising at least one aromatic group is at position 2, position 4 and/or position 5 of the peptidomimetic. According to further embodiments, the at least one AA comprising at least one aromatic moiety is at position 2, position 4 and/or position 5 of the peptidomimetic. According to some specific embodiments, the at least one AA comprising at least one aromatic moiety is at position 5 of the peptidomimetic.

According to some embodiments, at least one of the AAs in the peptidomimetic is alanine, in L or D chirality, or a derivative thereof. According to some embodiments, the alanine derivative comprises at least one cyclic group in its side-chain. As used herein, the terms “alanine with at least a cyclic substituent” and “alanine with at least a cyclic moiety” refer to amino acids comprising alanine bonded at least to one or more cyclic groups, including the amino acid phenylalanine and modifications or derivatives thereof.

According to some embodiments, at least one of the AAs in the peptidomimetic is a phenylalanine derivative. According to some particular embodiments, the at least one phenylalanine derivative is in at least one at position 2, position 4, and/or position 5 of the peptidomimetic.

According to some embodiments, at least one of the AAs is L-glycine or a derivative thereof. According to some particular embodiments, the at least one L-glycine or derivative thereof is in at least one at position 1 and position 5 of the peptidomimetic.

According to some embodiments, at least one at position 2, position 4, and position 5 of the peptidomimetic is occupied by an AA comprising at least one cyclic group. According to some embodiments, at least one at position 2, position 4, and position 5 of the peptidomimetic is occupied by an AA comprising at least one aromatic moiety. According to some particular embodiments, at least one at position 4 and position 5 of the peptidomimetic is occupied by an AA comprising at least one aromatic moiety. According to further particular embodiments, position 5 of the peptidomimetic is occupied by an AA comprising at least one aromatic moiety.

According to some embodiments, the peptidomimetic or conjugate has a conformation selected from a linear conformation, linear confirmation comprising a cyclic chain or a cyclic conformation. According to some particular embodiments, the peptidomimetic has a cyclic conformation. As used herein, the term “cyclic peptidomimetic” refers to a peptidomimetic having a chemical structure including a ring or cycle formed by at least 3 covalently bonded amino acids. The cyclic peptidomimetic comprises at least one cycle-forming linker which forms a bond, referred to herein as a “cyclization bond”, between a first amino acid and a second amino acid within the same peptide.

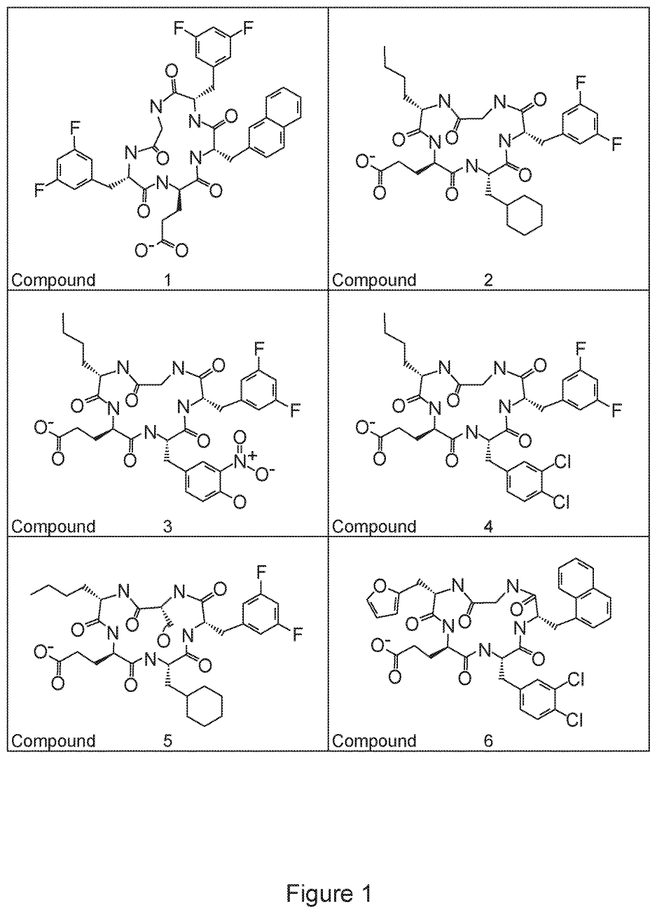

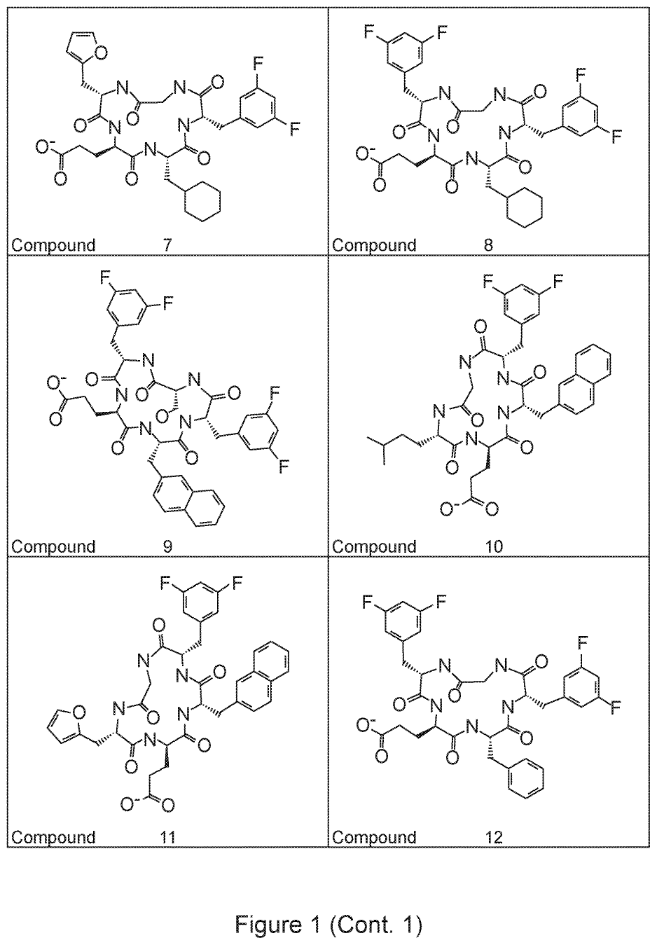

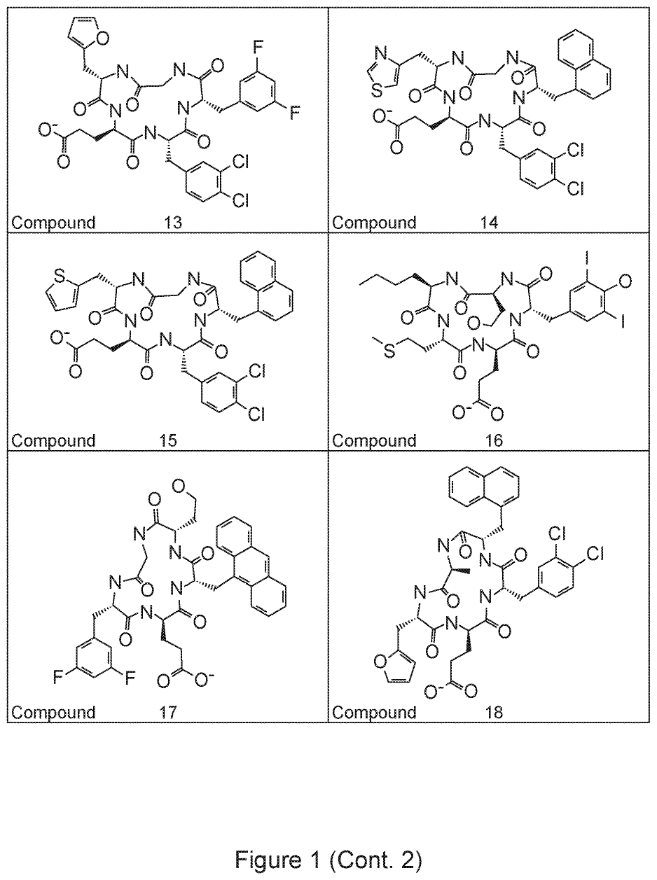

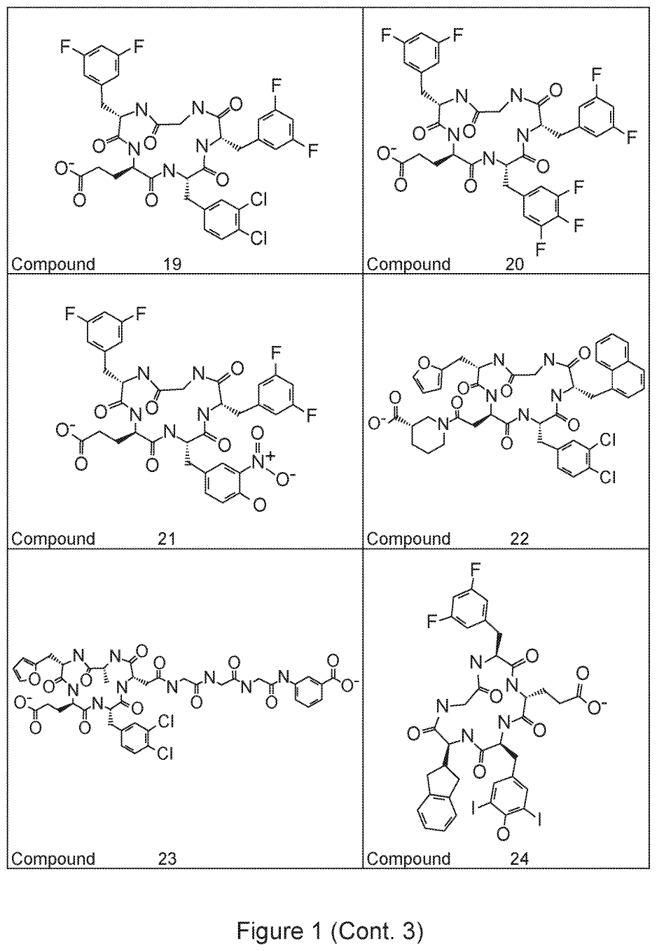

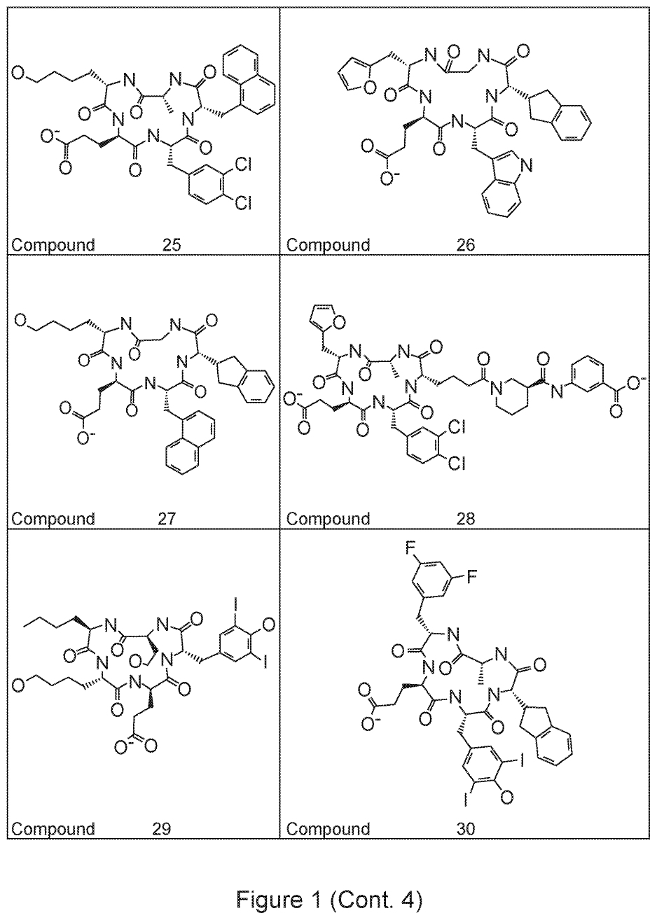

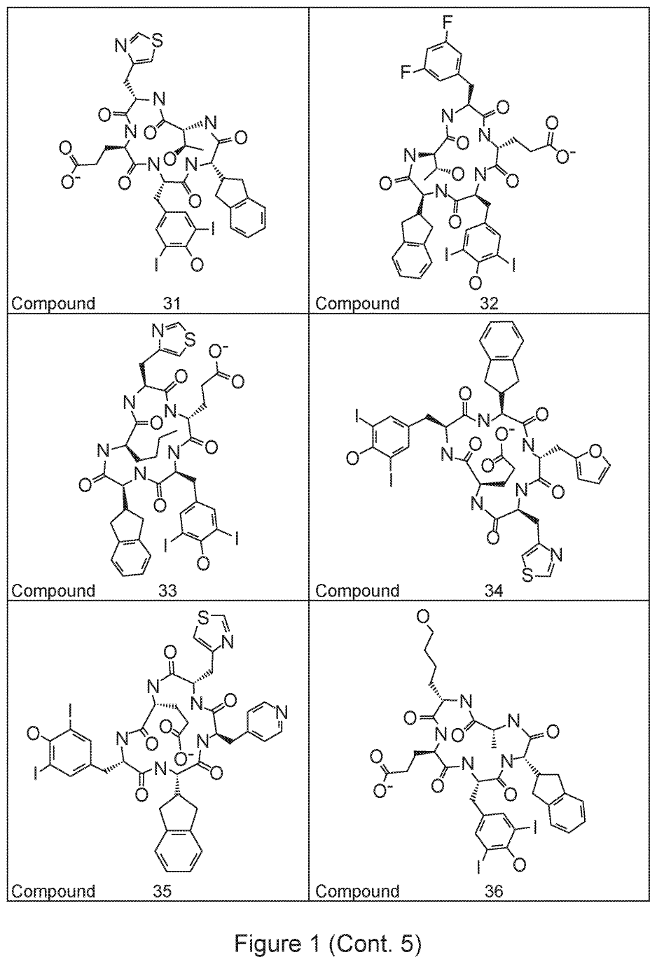

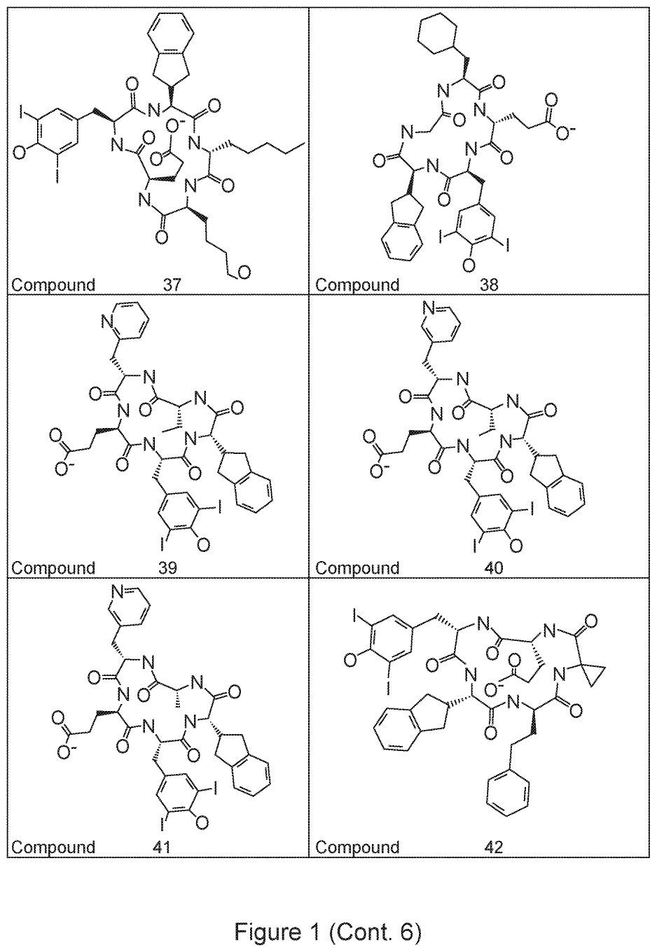

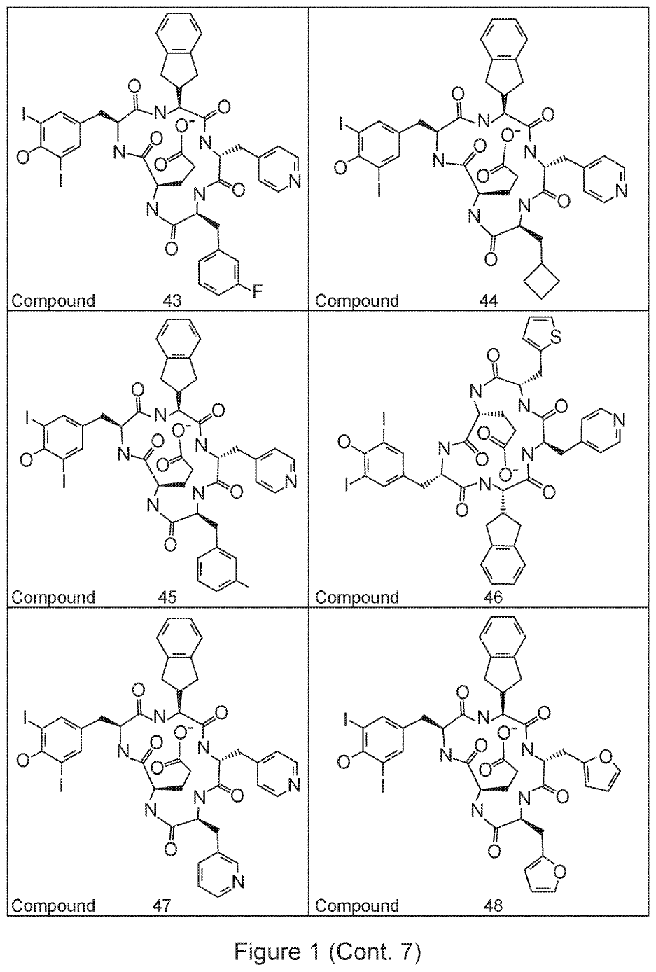

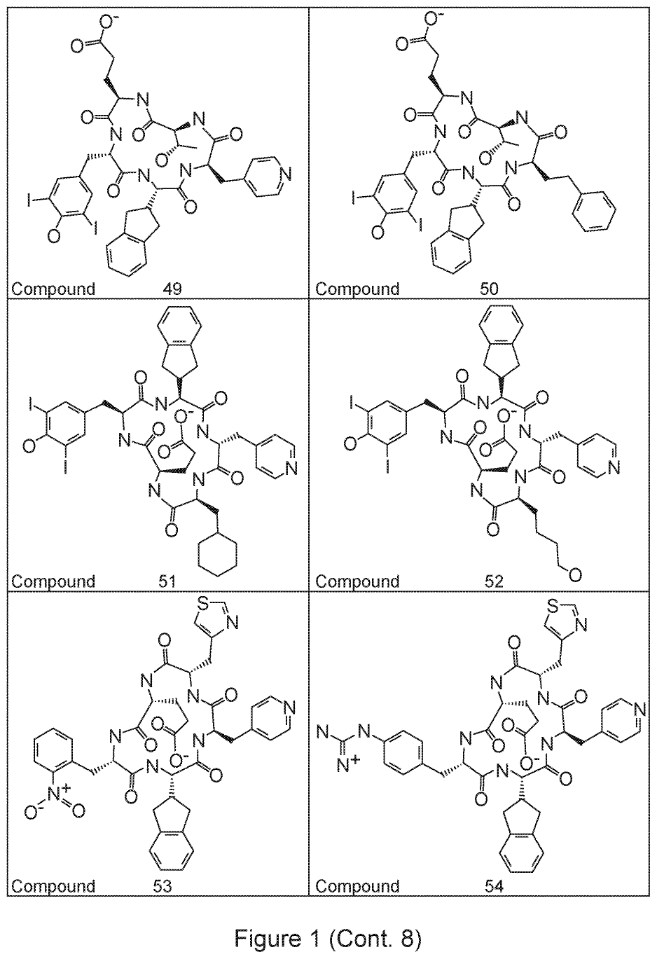

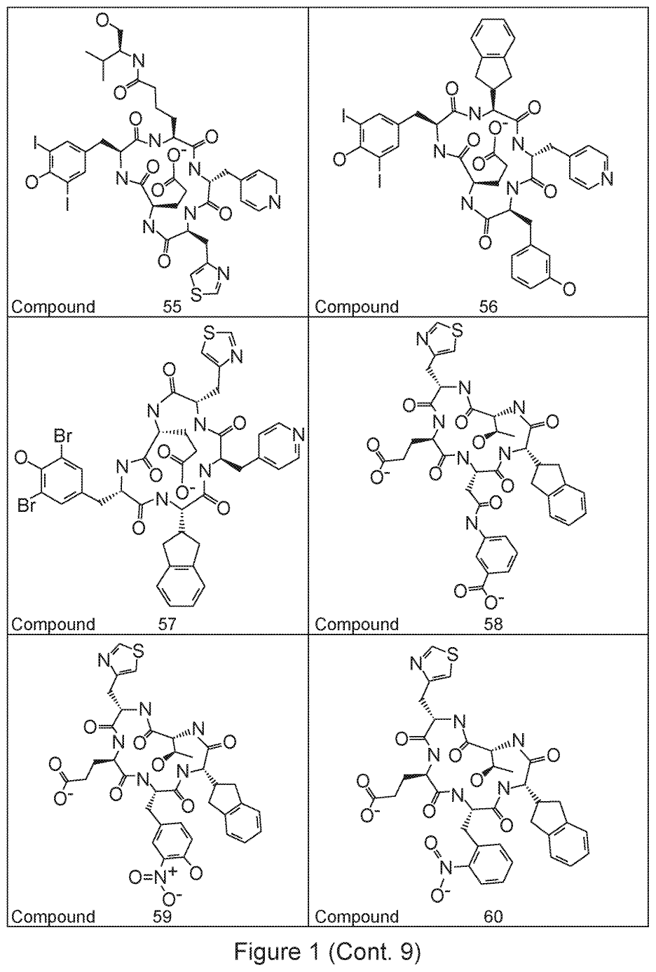

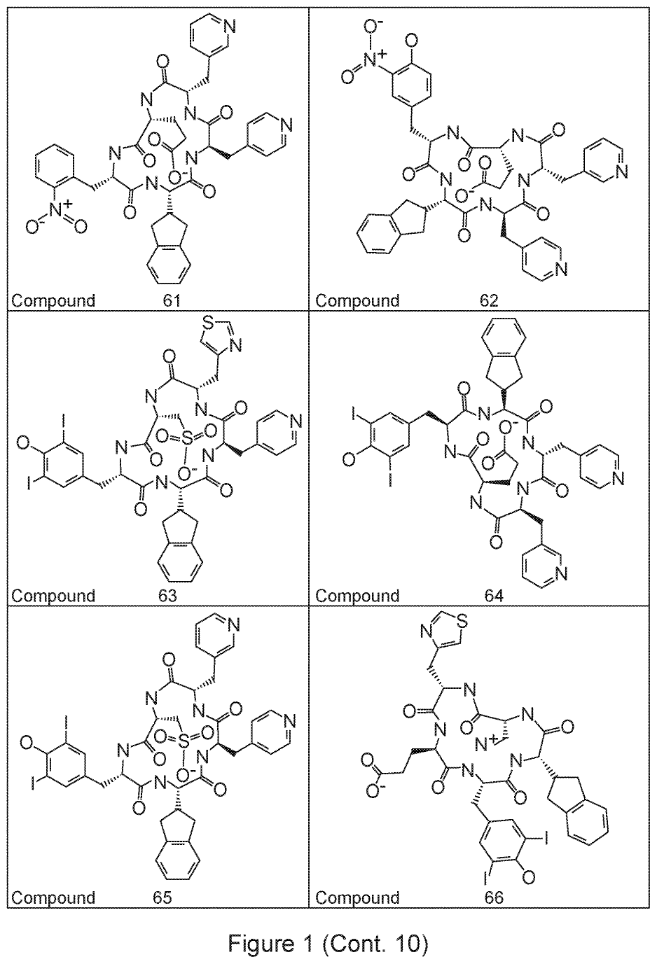

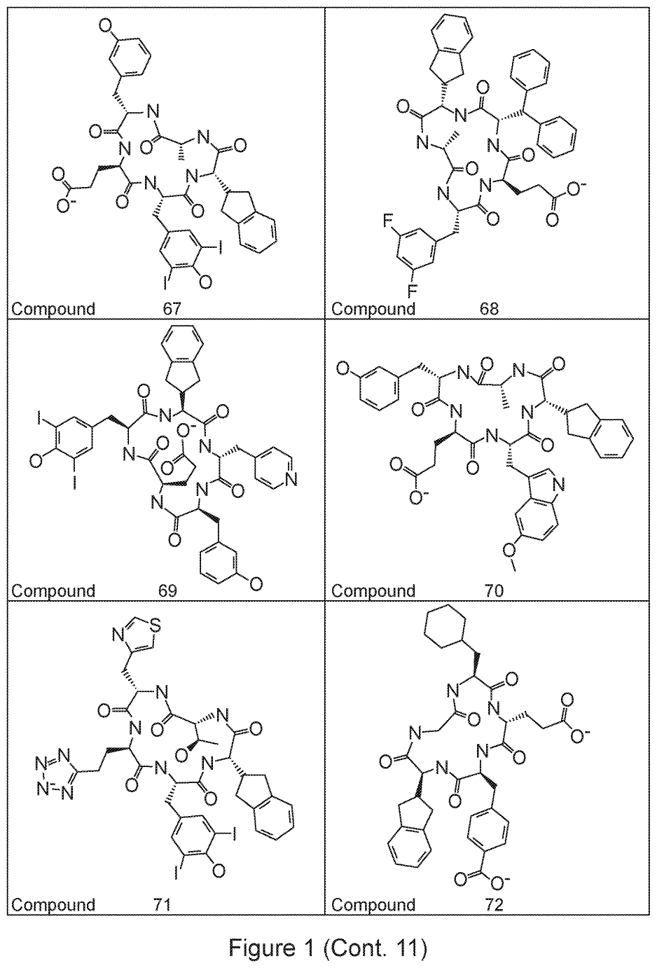

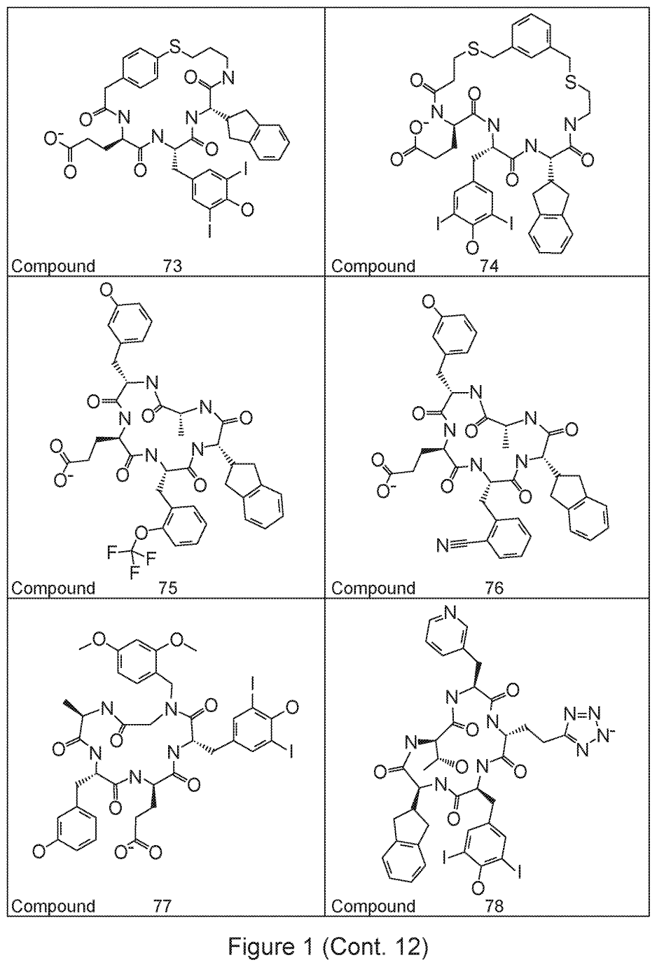

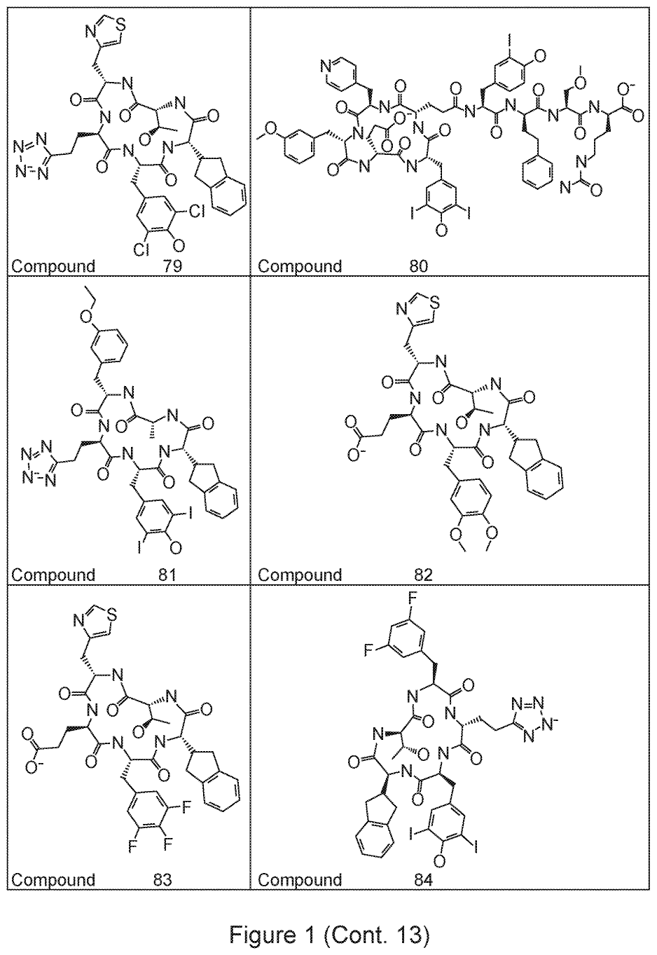

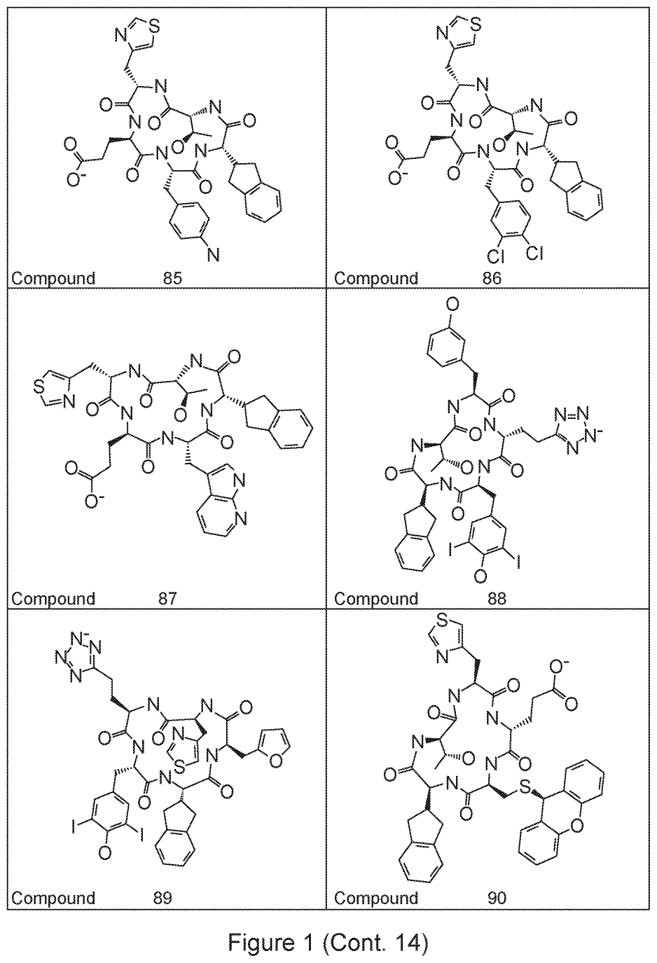

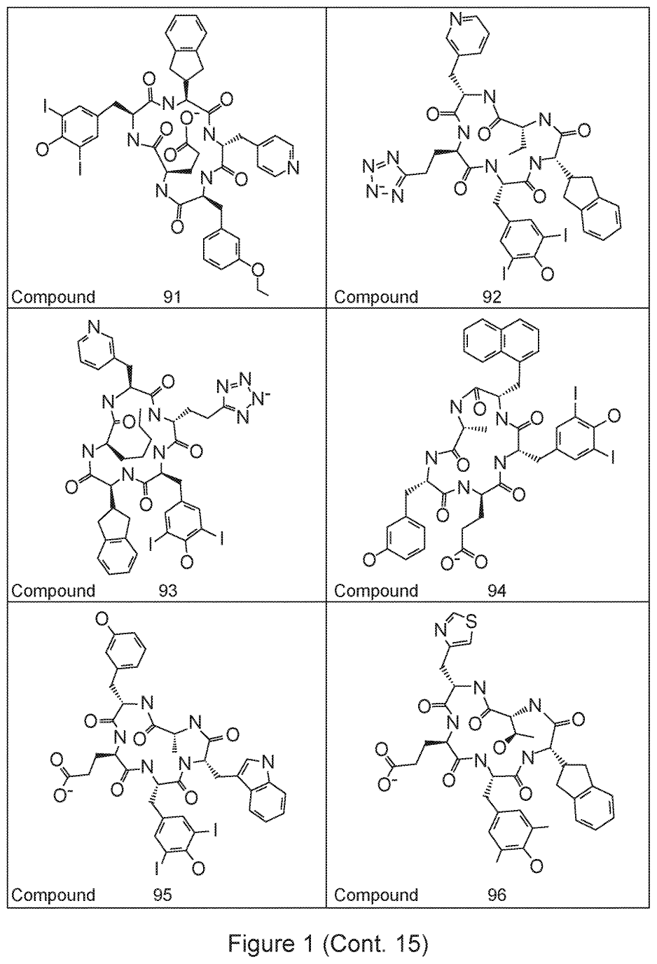

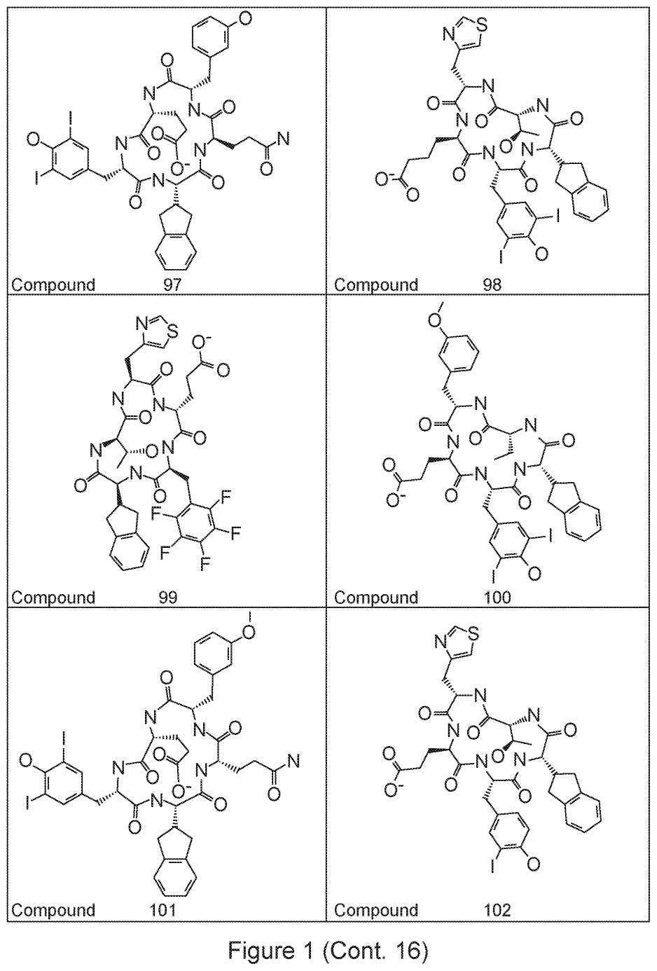

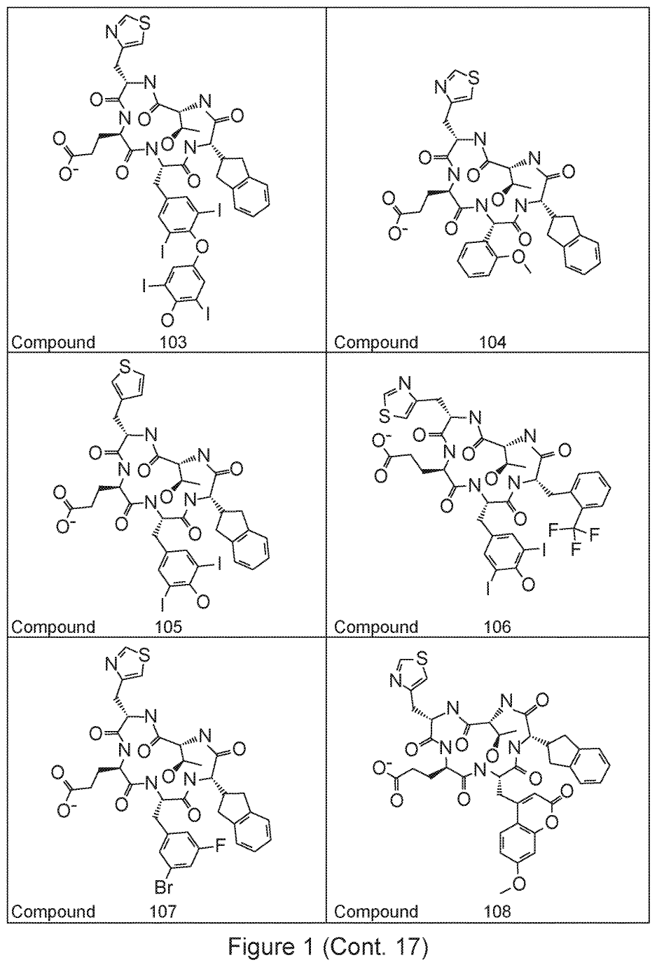

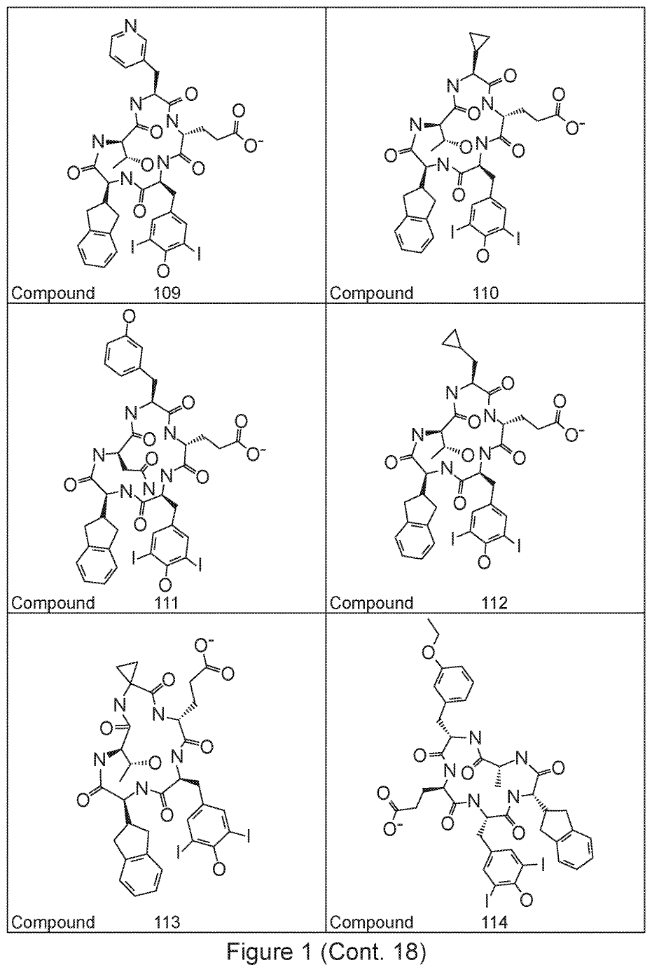

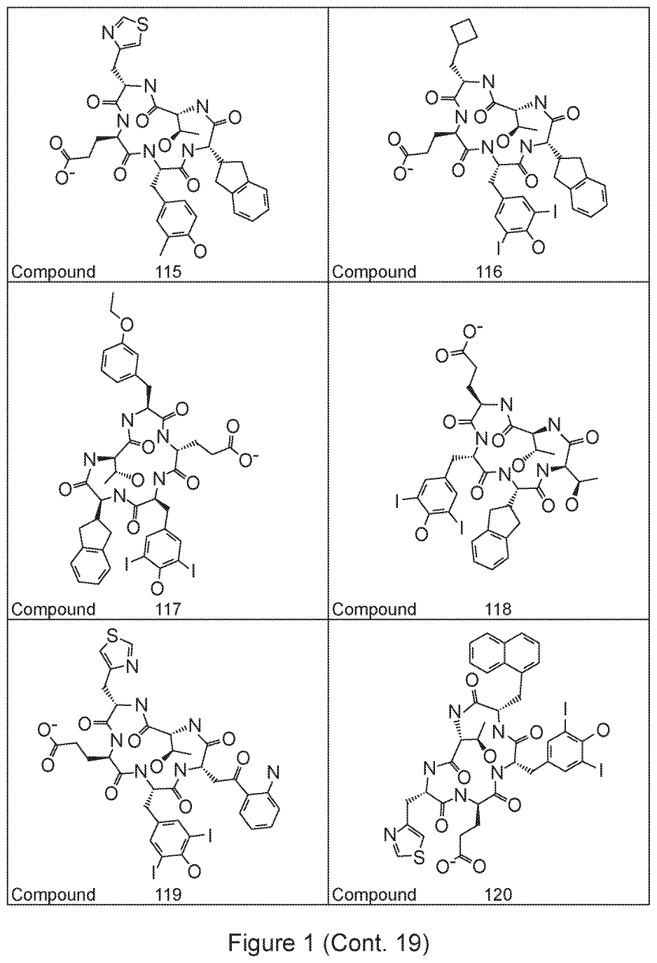

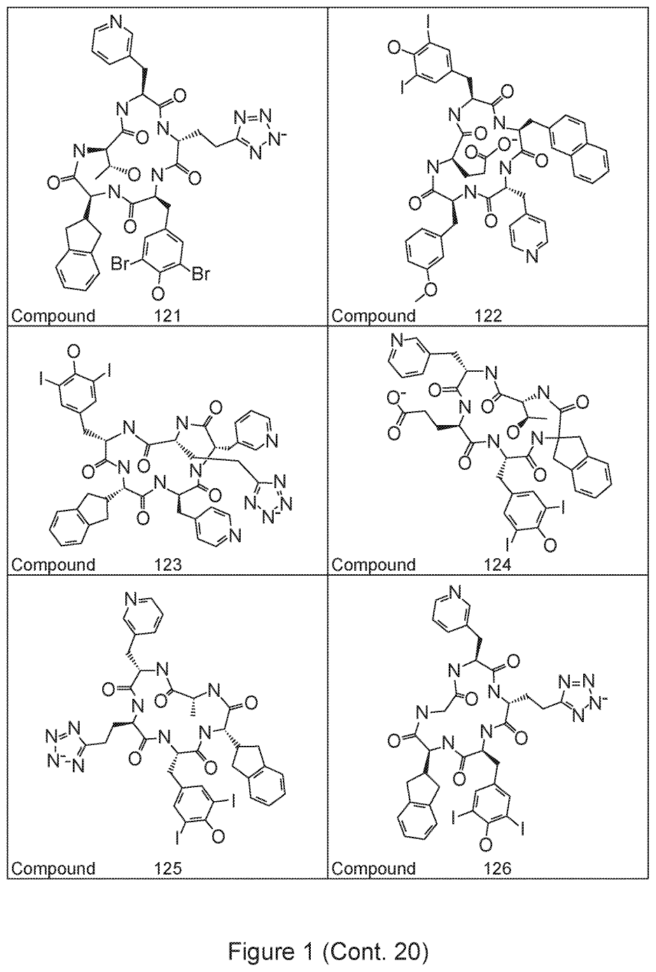

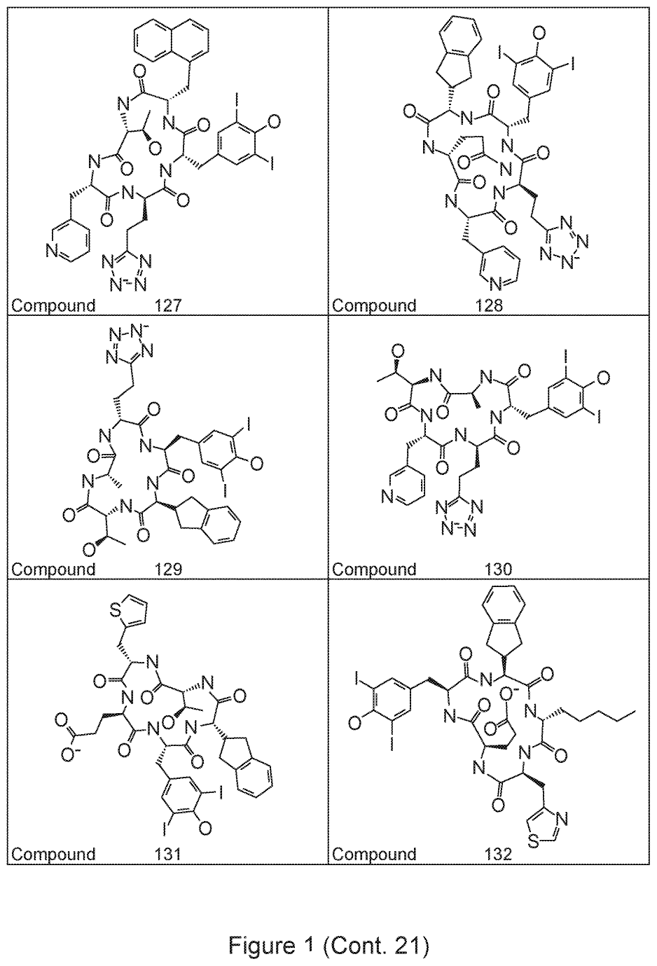

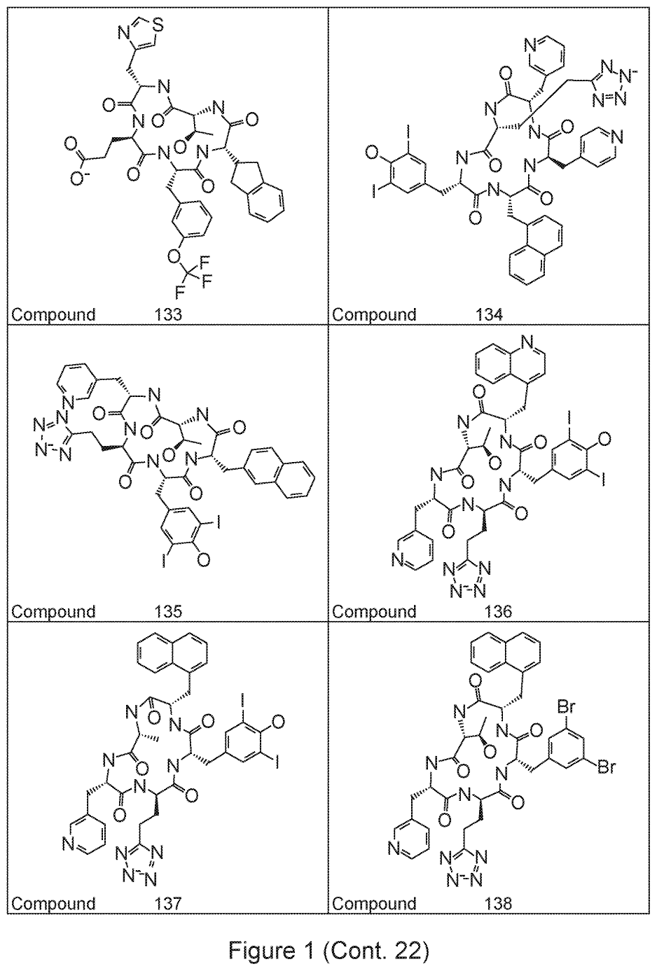

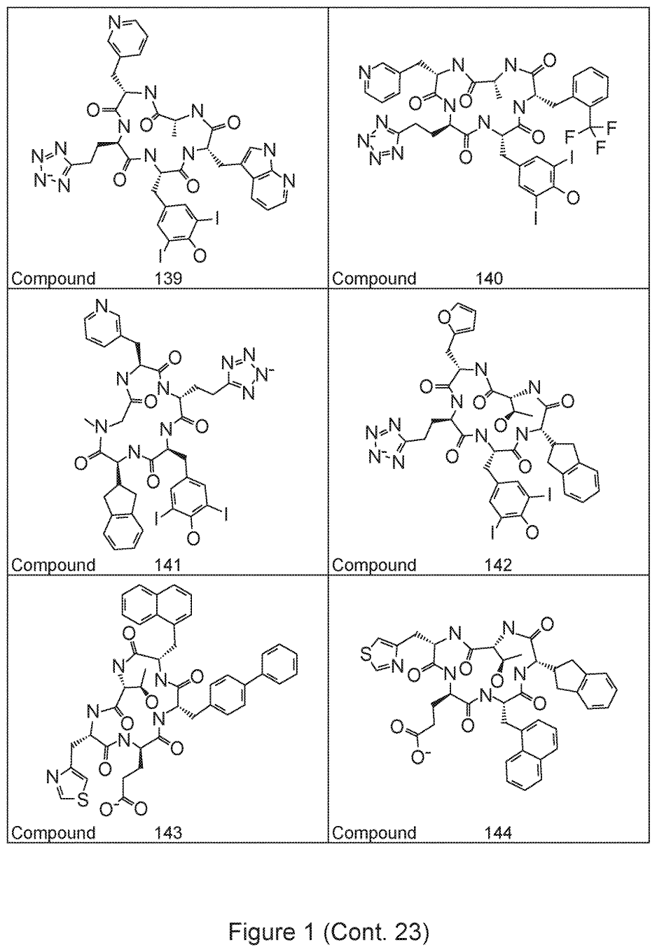

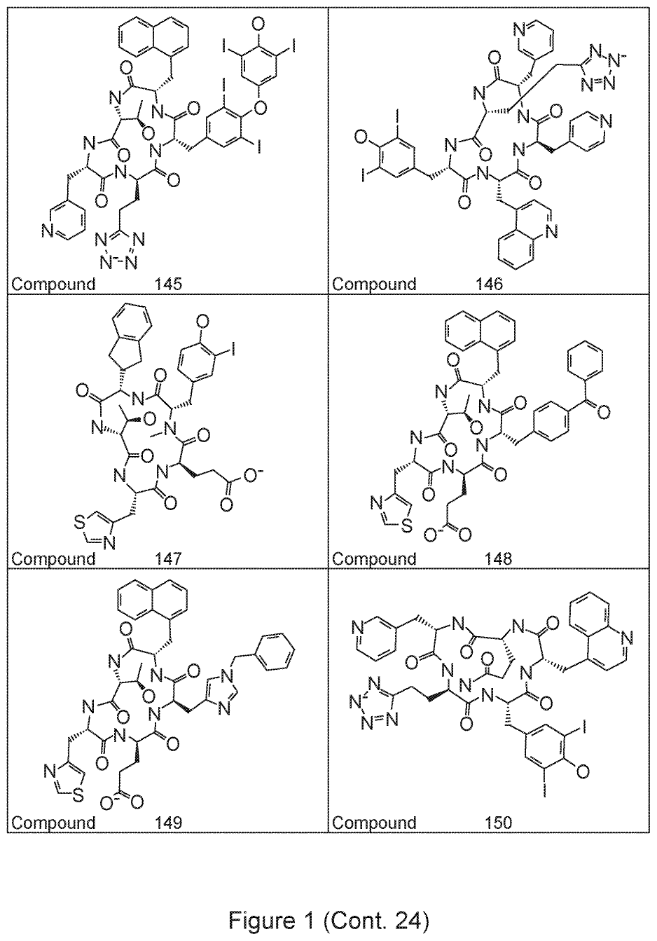

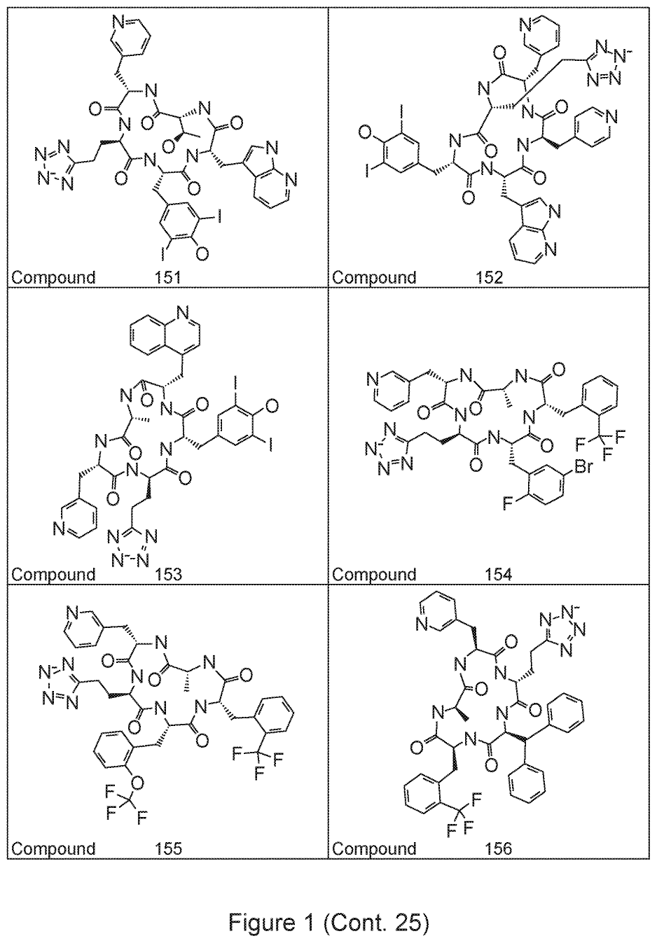

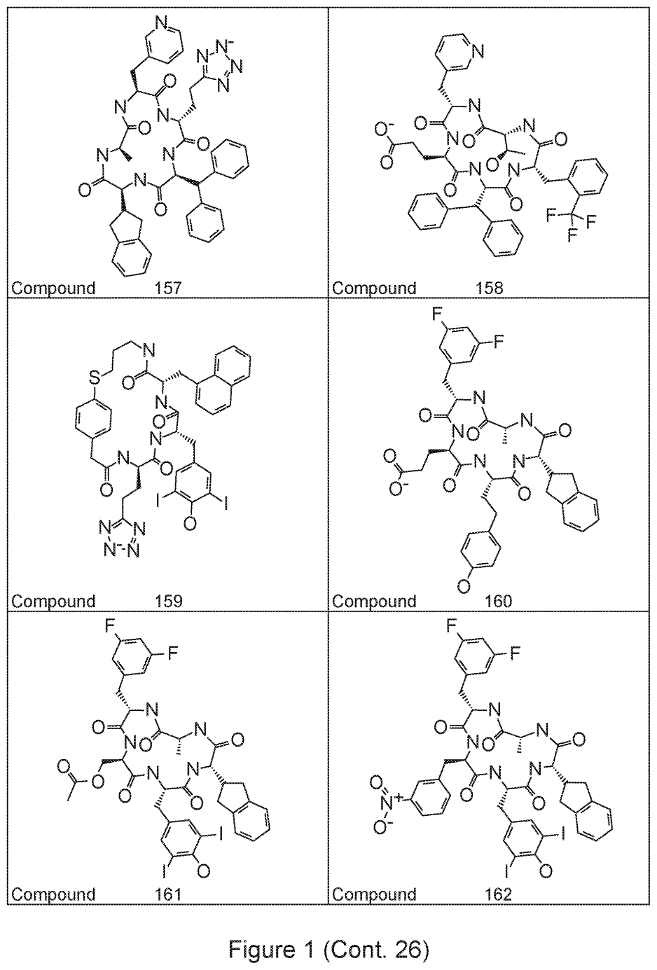

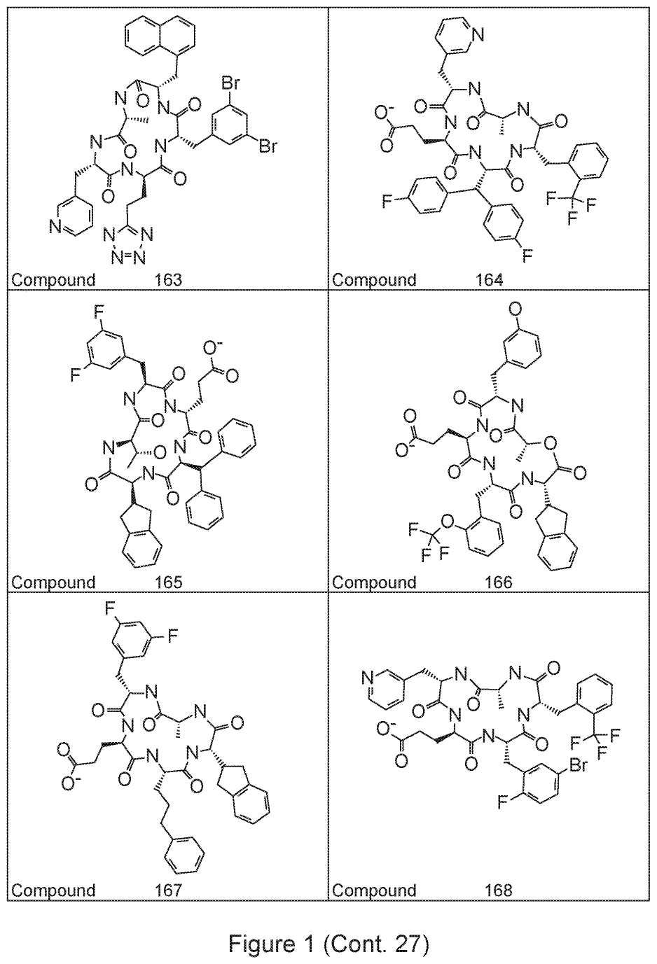

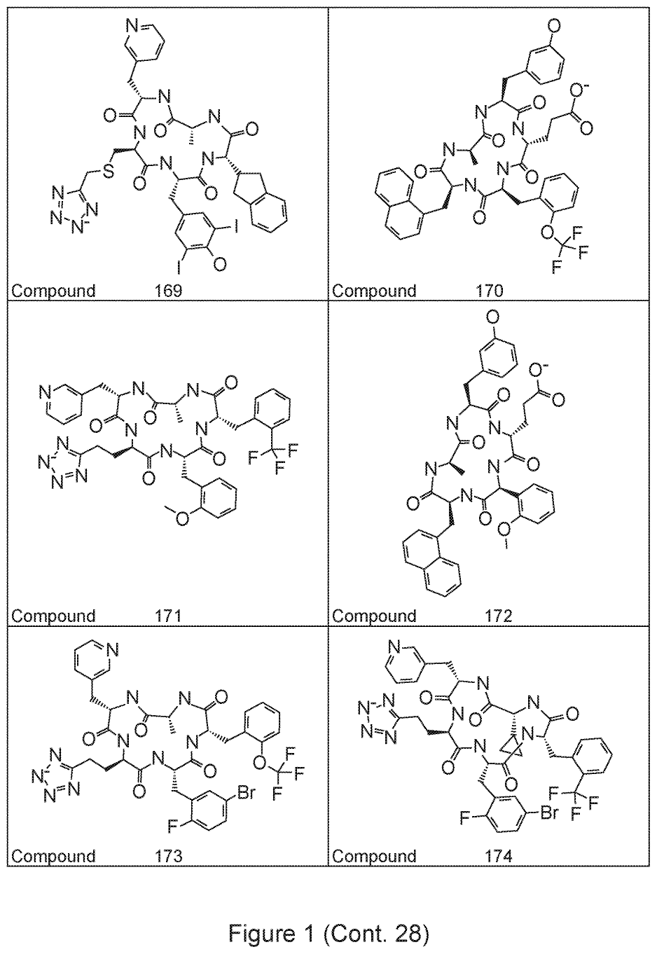

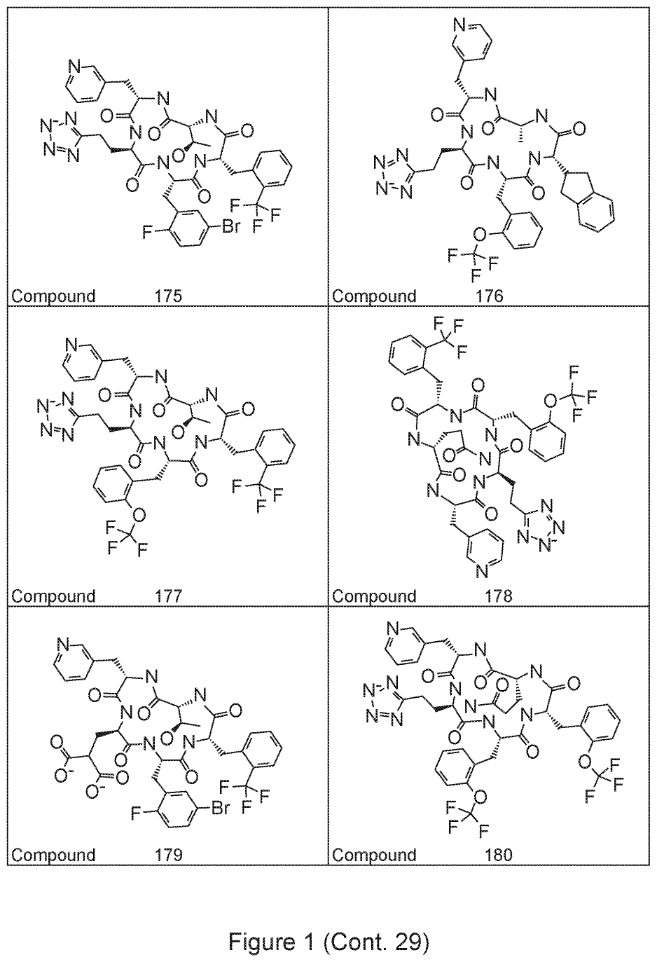

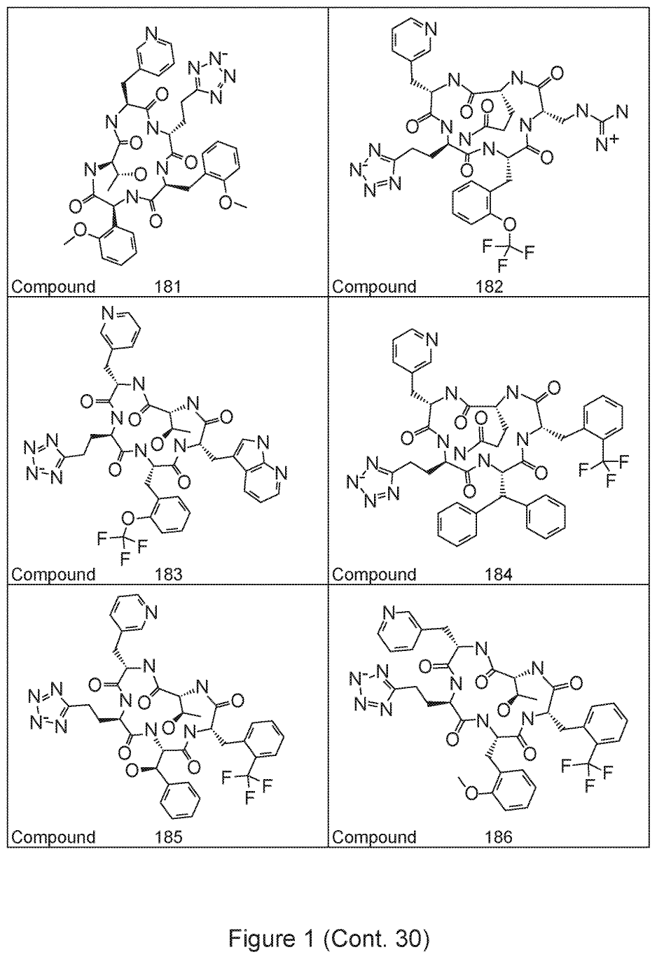

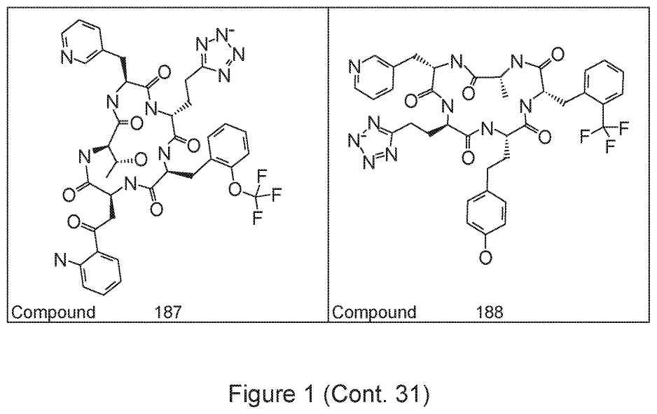

According to some embodiments, the cyclization bond if formed between the free carboxyl moiety of the last AA of the peptidomimetic and a free amino moiety of the first compound of the peptidomimetic. According to some particular embodiments, the cyclization bond if formed between the free carboxyl moiety of the last AA of the peptidomimetic and the free amino moiety of the first AA of the peptidomimetic. According to some embodiments, the cyclization bond is formed between the AA at position 1 of the peptidomimetic and the AA at position 5 of the peptidomimetic (herein 5 to 1 or 1 to 5 cyclization). It is to be understood, that according to some exemplary embodiments when the peptidomimetic consists of 5 AA (i.e., pentapeptide), the 5 to 1 cyclization is between position 1 and the last position of the peptidomimetic, i.e., head-to-tail cyclization (optionally referred to also as “C to N cyclization”). FIG. 1 provides the atomic structures of exemplary Compound Nos. 1-188.

Without being bound by any mechanism or theory of action, it is contemplated that the cyclic conformation of the peptidomimetics is advantageous for conferring enzymatic, pH and/or thermo-stability, intestinal permeability, and oral bioavailability while retaining, or even improving, the biological activity. Furthermore, cyclization leads to conformationally constrained of the peptides in preferred conformations for binding and activity. In particular, cyclic peptides eliminate terminal charges and are more spatially confined, thus reducing the enzyme mediated degradation propensity and lowering the loss of conformational entropy upon binding.

### Position 1 of the Peptidomimetic

According to some embodiments, position 1 is occupied by an AA selected from the group consisting of Gly, D-Ser, D-Nle, D-Ala, D-aThr, beta-(2-furyl)-D-Ala, D-Pal(4), D-Nle(Me), D-Abu, D-hPhe, D-Dap, 3-Chloropropylamine, Cysteamine, D-Gln, Nme-Gly, and D-Gly(cPr). Each possibility represents a separate embodiment of the invention. According to some embodiments position 1 is occupied by an AA having D-chirality. According to some embodiments, the AA having D-chirality is selected from D-Ala, D-Abu, D-Nle, D-Nle(Me), D-Pal(4), beta-(2-furyl)-D-Ala, D-hPhe, D-Ser, D-aThr, D-Gln, D-Gly(cPr), and D-Dap. Each possibility represents a separate embodiment of the invention.

According to some embodiments, position 1 of the peptidomimetic is occupied by an AA having a hydrophobic aliphatic moiety. According to some embodiments, the AA having a hydrophobic aliphatic moiety is selected from D-Ala, D-Abu, D-Nle, and D-Nle(Me). Each possibility represents a separate embodiment of the invention.

According to some embodiments, position 1 of the peptidomimetic is occupied by an AA having an aromatic moiety. According to some embodiments, the AA having an aromatic moiety is selected from D-Pal(4), beta-(2-furyl)-D-Ala, and D-hPhe. Each possibility represents a separate embodiment of the invention.

According to some embodiments, position 1 of the peptidomimetic is occupied by an AA having a hydrophilic moiety. According to some embodiments, the AA having a hydrophilic moiety is selected from D-aThr, D-Ser, D-Gln, and D-Dap. Each possibility represents a separate embodiment of the invention.

According to some embodiments, the amino acid at position 1 of the peptidomimetic is selected from the group consisting of D-Ala, D-aThr, D-Pal(4), D-Gln, D-Abu, D-Gly(cPr), and D-Nle(Me). According to some embodiments, the AA at position 1 of the peptidomimetic is selected from D-Abu, D-Nle(Me), D-Ala, D-aThr, Gly and D-Pal(4). According to some particular embodiments, the AA at position 1 is selected from the artificial AAs D-Abu, D-Nle(Me), D-Ala, and D-aThr. According to further specific embodiments, the AA at position 1 is selected from D-Abu and D-Nle(Me). Each possibility represents a separate embodiment of the invention.

According to some embodiments, position 1 of the peptidomimetic is occupied by an AA which is alanine or a derivative thereof having at least a cyclic substituent. According to some embodiments, alanine or the derivative thereof having at least a cyclic substituent is selected from D-Ala, D-Pal(4), beta-(2-furyl)-D-Ala, and D-hPhe. According to some particular embodiments, the AA is selected from D-Ala and D-Pal(4).

According to some embodiments, the peptidomimetic includes the AA D-Ala at position 1. According to some additional embodiments, the peptidomimetic includes the AA D-Pal(4) at position 1. According to some further embodiments, the peptidomimetic includes the AA D-aThr at position 1. According to still other embodiments, the peptidomimetic includes the AA Gly at position 1.

### Position 2 of the Peptidomimetic

According to some embodiments, position 2 is occupied by an amino acid selected from the group consisting of: Pal(3), Phe(3-OEt), Phe(3-OMe), Phe(3-5-di-F), Nle, beta-(2-furyl)-Ala, hLeu, Tza, 2Thi, 3Thi, Met, L-Nle(6-OH), Cha, Pal(2), Ac3c, Phe(3-F), Ala(cBu), Phe(3-Me), aThr, Phe(3-OH), Gly(cPr), Ala (cPr), Ala, and beta-(2-furyl)-D-Ala. Each possibility represents a separate embodiment of the invention. According to some embodiments, the amino acid at position 2 of the peptidomimetic is selected from the group consisting of Pal(3), Phe(3-OEt), 3-5-difluoro-phenylalanine (Phe(3-5-di-F)), and Phe(3-OMe). Each possibility represents a separate embodiment of the invention. According to some embodiments, the amino acid at position 2 of the peptidomimetic is Pal(3).

According to some embodiments, position 2 of the peptidomimetic is occupied by an AA comprising at least one group selected from an aromatic moiety, an aliphatic moiety, and a cyclo-aliphatic moiety.

According to some embodiments, position 2 of the peptidomimetic is occupied by an AA comprising a six-membered aromatic moiety. According to some embodiments, the AA comprising a six-membered aromatic moiety is selected from Phe(3-F), Phe(3-5-di-F), Phe(3-OH), Phe(3-OMe), Phe(3-OEt), Phe(3-Me), Pal(3) and Pal(2).

According to some embodiments, position 2 of the peptidomimetic is occupied by an AA comprising a five membered ring. According to some embodiments, the AA comprising a five membered ring is selected from 2Thi, Tza, 3Thi, and beta-(2-furyl)-Ala.

According to some embodiments, position 2 of the peptidomimetic is occupied by an AA comprising a cyclo-aliphatic moiety. According to some embodiments, the AA comprising a cyclo-aliphatic moiety is selected from Ac3c, Gly(cPr), Ala (cPr), Ala(cBu) and Cha.

According to some embodiments, position 2 of the peptidomimetic is occupied by an AA comprising a linear aliphatic side-chain. According to some embodiments, the AA comprising a linear aliphatic side-chain is selected from Met, Nle, hLeu and Nle(6-OH).

According to some embodiments, position 2 of the peptidomimetic is occupied by an artificial AA having a small, non-cyclic side-chain. According to some embodiments, the AA having a small, non-cyclic side chain is selected from Ac3c and aThr. According to some particular embodiments, the peptidomimetic includes an AA having a voluminous side chain at position 1, i.e., a side chain including at least one cyclic group, and an AA having a small, non-cyclic side chain at position 2. According to some embodiments, the AA having a voluminous side chain at position 2 is selected from D-hPhe and D-Pal(4).

According to some embodiments, the peptidomimetic includes at position 2 an artificial AA which is an alanine with at least a cyclic group, selected from the group consisting of beta-(2-furyl)-Ala, Tza, Cha, Pal(3), Pal(2), Ala(cBu), Phe(3-F)), Phe(3-Me), Phe(3-OH), Phe(3-OMe), and Phe(3-5-di-F). According to some embodiments, the alanine with at least a cyclic group is selected from Tza, Phe(3-5-di-F), Phe(3-OH), Pal(3), and beta-(2-furyl)-Ala.

According to some embodiments, the peptidomimetic includes at position 2 a non-DNA-encoded AA which is a derivative of phenylalanine selected from the group consisting of Phe(3-F)), Phe(3-Me), Phe(3-OH), Phe(3-OMe), Phe (OEt), and Phe(3-5-di-F). According to some specific embodiments, the derivative of phenylalanine includes at least one fluoro moiety. According to further specific embodiments, the AA is Phe(3-5-di-F).

According to some embodiments, the peptidomimetic includes the AA Tza at position 2. According to some embodiments, the peptidomimetic includes the AA beta-(2-furyl)-Ala at position 2.

According to some embodiments, the peptidomimetic includes at position 2 an AA which is L-Norleucine (Nle), or a derivative thereof selected from L-Nle(6-OH) and hLeu. According to some particular embodiments, the AA is Nle. According to further particular embodiments the AA is L-Nle(6-OH).

According to some embodiments, the peptidomimetic includes at position 2 an AA which is alanine bonded to a pyridyl moiety, selected from Pal(2) and Pal(3). According to some particular embodiments, the alanine bonded to a pyridyl moiety is Pal(3).

According to some embodiments, position 2 of the peptidomimetic is occupied by an artificial AA selected from the group consisting of Phe(3-OMe), Phe(3-OH), 2Thi, Ala(cBu), Tza, Cha, Phe(3-F), Phe(3,5-F), Pal(3), Ac3c, aThr, and hLeu. According to some particular embodiments, the AA is selected from the group consisting of Phe(3-OMe), Phe(3-OH), 2Thi, Ala(cBu), Tza and Cha. According to some embodiments, the AA at position 2 of the peptidomimetic is Phe(3-OMe).

Without being bound by theory or mechanism of action, it is contemplated that occupying position 2 of the peptidomimetic with the AA Phe(3-OEt) is particularly advantageous, as this AA is modelled to effectively fill the hydrophobic IL-17 pocket space, while receiving a hydrogen bond.

### Position 3 of the Peptidomimetic

According to some embodiments, position 3 is occupied by an AA selected from the group consisting of D-Abu(tetrazol-5-yl), D-Cys(-Methyl-2H-tetrazole)), D-Glu, D-Cys (O₃H), D-Aad, D-Ser (Ac), D-Phe(3-NO₂), D-Asp-[-1Nip], and D-Gla. Each possibility represents a separate embodiment of the invention. According to some embodiments, position 3 is occupied by an AA having D-chirality. According to some embodiments, the AA having D-chirality is selected from D-Abu(tetrazol-5-yl), D-Glu, D-Gla, D-Cys (O₃H), and D-Aad. Each possibility represents a separate embodiment of the invention. According to some embodiments, the AA having D-chirality is selected from D-Abu(tetrazol-5-yl), D-Cys(-Methyl-2H-tetrazole), and D-Glu. Each possibility represents a separate embodiment of the invention.

According to some embodiments, position 3 is occupied by an AA having an anionic residue and/or a cluster of negative atomic partial charges at pH 7.4. According to some embodiments, the AA having an anionic residue and/or a cluster of negative atomic partial charges at pH 7.4 is selected from D-Glu, D-Gla, D-Cys (O₃H), and D-Aad, and D-Abu(tetrazol-5-yl). Each possibility represents a separate embodiment of the invention. According to some embodiments, the amino acid at position 3 is D-Cys (O₃H). According to some embodiments, the amino acid at position 3 is D-Glu.

According to the principles of the present invention, one of the criteria used by the algorithm employed for identifying peptidomimetics having improved bioavailability is minimizing complete charges on the peptidomimetics, while dispersing the charge over several atoms as partial charges. As is well-established in the art, peptidomimetics and other peptides that include charged moieties have reduced absorbance by the intestines, thereby limiting their use as oral drugs. Tetrazoles are neutral in the pH of the gastrointestinal tract, thereby improving absorbance by the intestines, as is exemplified by orally available small molecule drugs that contain tetrazoles, such as losartan and candesartan. According to some embodiments, the anionic moiety at pH 7.4 is replaced with a neutral one which contains atoms of a negative partial charge. According to some embodiments, the amino acid at position 3 is D-Ser (Ac). According to some embodiments, the amino acid at position 3 is D-Phe(3-NO₂). According to some embodiments, the amino acid at position 3 has a tetrazole, providing a cluster of negative atomic partial charges. According to some embodiments, the negative atomic partial charges are defined by the AMBER Force Field. Negative partial charges are caused by the uneven distribution of electrons in chemical bonds, with bonds between a strong electronegative atom and less electronegative atoms causing a negative partial charge on the more electronegative atom. According to some embodiments, the amino acid at position 3 of the peptidomimetic is selected from D-Abu(tetrazol-5-yl) and D-Cys(-Methyl-2H-tetrazole). Each possibility represents a separate embodiment of the invention. According to other embodiments, the amino acid at position 3 of the peptidomimetic is D-Abu(tetrazol-5-yl).

According to some embodiments, position 3 is occupied by an AA having D-chirality and an anionic residue and/or a cluster of negative atomic partial charges at pH 7.4. According to some embodiments, the AA having D-chirality and an anionic residue and/or a cluster of negative atomic partial charges at pH 7.4 is selected from D-Glu, D-Gla, D-Cys (O₃H), D-Aad, and D-Abu(tetrazol-5-yl). Each possibility represents a separate embodiment of the invention.

Without being bound by theory or mechanism of action, it is contemplated that the effectivity of the residues occupying position 3 is at least partially due to an electrostatic interaction thereof with the lysine at position 114 in chain A of the IL-17 protein.

### Position 4 of the Peptidomimetic

According to some embodiments, position 4 of the peptidomimetic is occupied by an amino acid selected from the group consisting of Phe(2-OCF₃), Dip, Phe(2-OMe), Phe (bR-OH), Phe(3-5-di-Br), 2-Nal, Cha, Tyr(3-NO₂), Phe(3-4-Cl), Ala (9-Anth), Phe(3,4,5-F), Trp, 1-Nal, Phe(2-NO₂), Phe(4-guanidino), Tyr(3-5-di-Bromo), L-isoAsp-[-3Abz], 5-methoxy-tryptophan, 4-CarboxyPhe, Phe(2-CN), Tyr(3-5-di-Cl), Phe(3-4-diOMe), Phe(4-NH₂), L-(7-azaTrp), Cys(Xan), Tyr(3-5-di-Me), Phe(F₅), Tyr(3-Iodo), L-thyroxine, Phg(2-OMe), 3-Bromo-5-fluoro-phenylalanine, beta-(7-methoxy-coumarin-4-yl)-Ala, Tyr(3-Me), Bip, Nme-Tyr(3-Iodo), Bpa, D-His(1-Bn), hTyr, 2-Amino-3,3-bis(4-fluorophenyl) propanoic acid, Phe(3-4-5-F), and Nva(Ph). Each possibility represents a separate embodiment of the invention. According to some embodiments, position 4 of the peptidomimetic is occupied by an amino acid selected from the group consisting of Tyr(3-5-di-Iodo), 5-Bromo-2-fluoro-phenylalanine, Phe(2-OCF₃), Dip, Phe(2-OMe), and Phe(3-5-di-Br). Each possibility represents a separate embodiment of the invention. According to some embodiments, the amino acid at position 4 of the peptidomimetic is selected from Tyr(3-5-di-Iodo) and 5-Bromo-2-fluoro-phenylalanine. Each possibility represents a separate embodiment of the invention.

According to some embodiments, position 4 of the peptidomimetic is occupied by an AA having L-chirality. According to some embodiments, the AA having L-chirality is selected from Tyr(3-5-di-Iodo), Tyr(3-5-di-Bromo) Tyr(3-5-di-Cl), Phe(2-OCF₃), Phe(2-NO₂), Tyr(3-NO₂), Phe(4-guanidino), 4-CarboxyPhe, Phe(2-CN), Phe(3-4-Cl), Phe(3,4,5-F), Pal(2), Dip, 1-Nal, 2-Nal, Trp, 5-methoxy-tryptophan, Ala (9-Anth), hTyr, Nva(Ph), Phe (bR-OH) and Cha. Each possibility represents a separate embodiment of the invention.

According to some embodiments, position 4 of the peptidomimetic is occupied by an AA comprising at least one aromatic moiety. According to some embodiments, the AA having at least one aromatic moiety is selected from Tyr(3-5-di-Iodo), Tyr(3-5-di-Bromo) Tyr(3-5-di-Cl), L-Phe(2-OCF₃), Phe(2-NO₂), Tyr(3-NO₂), Phe(4-guanidino), 4-CarboxyPhe, Phe(2-CN), Phe(3-4-Cl), Phe(3,4,5-F), Pal(2), Dip, 1-Nal, 2-Nal, Trp, 5-methoxy-tryptophan, Ala (9-Anth), Phg(2-OMe), Phe(4-NH₂), Phe(F₅), L-(7-azaTrp), hTyr, Nva(Ph), Phe (bR-OH) and Cha. Each possibility represents a separate embodiment of the invention.

According to some embodiments, position 4 of the peptidomimetic is occupied by an AA having L-chirality and at least one aromatic moiety. According to some embodiments, the AA having L-chirality and at least one aromatic moiety is selected from Tyr(3-5-di-Iodo), Tyr(3-5-di-Bromo) Tyr(3-5-di-Cl), Phe(2-OCF₃), Phe(2-NO₂), Tyr(3-NO₂), Phe(4-guanidino), 4-CarboxyPhe, Phe(2-CN), Phe(3-4-Cl), Phe(3,4,5-F), Pal(2), Dip, 1-Nal, 2-Nal, Trp, 5-methoxy-tryptophan, Ala (9-Anth), and Cha. Each possibility represents a separate embodiment of the invention.

According to some embodiments, position 4 of the peptidomimetic is occupied by an AA comprising a single aromatic moiety. According to some embodiments, the AA having a single aromatic moiety is selected from Tyr(3-5-di-Iodo), Tyr(3-5-di-Bromo) Tyr(3-5-di-Cl), L-Phe(2-OCF₃), Phe(2-NO₂), Tyr(3-NO₂), Phe(4-guanidino), 4-CarboxyPhe, Phe(2-CN), Phe(3-4-Cl), Phe(3,4,5-F), Pal(2), Phg(2-OMe), Phe(4-NH₂), Phe(F₅), and Cha. Each possibility represents a separate embodiment of the invention.

According to some embodiments, position 4 of the peptidomimetic is occupied by an AA having more than one aromatic moiety. According to some embodiments, the AA having more than one aromatic moiety is selected from Dip, 1-Nal, 2-Nal, Trp, 5-methoxy-L-tryptophan, L-(7-azaTrp), and Ala (9-Anth). Each possibility represents a separate embodiment of the invention.

According to some embodiments, position 4 of the peptidomimetic is occupied by an AA having L-chirality and a modified 6-membered aromatic moiety. According to some embodiments, the AA having L-chirality and a modified 6-membered aromatic moiety is selected from Tyr(3,5-di-Iodo), Tyr(3-5-di-Bromo), Tyr(3-5-di-Cl), Phe(2-OCF₃), Phe(3,4,5-F), Phe(2-NO₂), Tyr(3-NO₂), Phe(4-guanidino), Phe(3,4-di-Cl), Phe(2-CN), Phe(4-NO₂), 4-CarboxyPhe, and 5-methoxy-tryptophan. According to some particular embodiments, the AA having L-chirality and a modified 6-membered aromatic moiety is selected from Tyr(3,5-di-I), Tyr(3-5-di-Bromo), Phe(2-OCF₃), Phe(2-NO₂), Tyr(3-NO₂), and Phe(4-guanidino).

According to some embodiments, position 4 of the peptidomimetic is occupied by an AA having a modified 6-membered aromatic moiety. According to some embodiments, the AA having a modified 6-membered aromatic moiety is selected from Tyr(3,5-di-Iodo), Tyr(3-5-di-Bromo), Tyr(3-5-di-Cl), Phe(2-OCF₃), Phe(3,4,5-F), Phe(2-NO₂), Tyr(3-NO₂), Phe(4-guanidino), Phe(3,4-di-Cl), Phe(2-CN), Phe(4-NO₂), 4-CarboxyPhe, and 5-methoxy-tryptophan. According to some particular embodiments, the AA having a modified 6-membered aromatic moiety is selected from Tyr(3,5-di-Iodo), Tyr(3-5-di-Bromo), Phe(2-OCF₃), Phe(2-NO₂), Tyr(3-NO₂), and Phe(4-guanidino).

According to some further embodiments, position 4 of the peptidomimetic is occupied by an AA which is a 3,5 di-halogen modified tyrosine. According to some embodiments, the AA comprising 3,5 di-halogen modified tyrosine is selected from hTyr, Tyr(3,5-di-Iodo), Tyr(3-5-di-Bromo) and Tyr(3-5-di-Cl).

According to some embodiments, the AA at position 4 of the peptidomimetic is selected from Tyr(3,5-di-Iodo), Tyr(3,5-di-Br), Phe(2-OCF₃), Phe(2-NO₂), Tyr(3-NO₂), Cha, Dip, Phe(4-guanidino), Phe(3,4-di-Cl), Phe(3,4,5-F), Phe(2-CN), Phe(4-NO₂), 4-CarboxyPhe, 5-methoxy-tryptophan and Ala (9-Anth). According to some particular embodiments, the AA at position 4 of the peptidomimetic is selected from Tyr(3,5-di-Iodo), Tyr(3,5-di-Bromo), Phe(2-OCF₃), Phe(2-NO₂), Tyr(3-NO₂), Cha, Dip, Phe(4-guanidino) and Ala (9-Anth). According to further particular embodiments, the AA at position 4 of the peptidomimetic is selected from Tyr(3,5-di-Iodo), Tyr(3,5-di-Bromo), Phe(2-OCF₃) and Phe(2-NO₂). According to further specific embodiments, the AA at position 4 of the peptidomimetic is selected from Tyr(3,5-di-Iodo) and Tyr(3,5-di-Bromo). According to some embodiments, the AA at position 4 of the peptidomimetic is Tyr(3,5-di-Bromo). According to some embodiments, the AA at position 4 of the peptidomimetic is Tyr(3,5-di-Iodo).

According to some embodiments, the peptidomimetic includes at position 4 a non-DNA-encoded AA which is a tyrosine derivative. According to some embodiments, the tyrosine derivative is selected from Tyr(3-5-di-Iodo), Tyr(3-NO₂), and Tyr(3-5-di-Bromo). According to some particular embodiments, the tyrosine derivative at position 4 is Tyr(3-5-di-Iodo). Each possibility represents a separate embodiment of the invention.

According to some embodiments, the peptidomimetic includes at position 4 an artificial AA which is an alanine derivative with at least a cyclic substituent. According to some embodiments, the alanine derivative with at least a cyclic substituent is selected from the group consisting of 2-Nal, 1-Nal, Ala (9-Anth), Cha, Dip, 4-carboxyPhe, L-Phe(2-OCF₃), Phe(2-CN), Phe(2-NO₂), Phe(4-guanidino), Phe(3-4-Cl), and Phe(3-4-5-F). According to some particular embodiments, the alanine derivative is selected from Cha, 2-Nal, 1-Nal, and Ala (9-Anth). Each possibility represents a separate embodiment of the invention.

According to some embodiments, the peptidomimetic includes at position 4 an artificial AA which is a phenylalanine derivative selected from the group consisting of Phe(3-4-Cl), Phe(2-NO₂), 4-carboxyPhe, Phe(2-OCF₃), Phe(2-CN), Phe(4-guanidino), and Phe(3-4-5-F). According to some particular embodiments, the phenylalanine derivative is Phe(3-4-Cl). Each possibility represents a separate embodiment of the invention.

Without being bound by theory or mechanism of action, it is contemplated that an advantageous function of the AA which occupies position 4 in the inhibition of IL-17A is space filling. Specifically, volumetric modifications of the aromatic rings of the AA generally show high p(IC₅₀) contributions, as measured at least by ELISA or AlphaLISA.

### Position 5 of the Peptidomimetic

According to some embodiments, position 5 of the peptidomimetic is occupied by an amino acid selected from the group consisting of Gly (indan-2-yl), Phe(2-CF₃), 1-Nal, 3-(4-Quinolyl)-alanine, L-(7-aza Trp), Phe(2-OCF₃), 2-Nal, Phe(3-5-di-F), Hse, L-Asp-[-GLY-GLY-GLY-3Abz], isoAad-[-1Nip-3Abz], isoAad-[-Val-ol], L-isoGlu-[-Tyr(3-I)-DhPhe-Ser (Me)-DCit], Trp, Asp(Ph(2-NH₂)), Aic, Ala, Phg(2-OMe), (Dmb)Gly, and Ala (guanidino). Each possibility represents a separate embodiment of the invention. According to some embodiments, position 5 of the peptidomimetic is occupied by an amino acid selected from the group consisting of Gly (indan-2-yl), Phe(2-CF₃), 1-Nal, 3-(4-Quinolyl)-alanine, L-(7-azaTrp), Phe(2-OCF₃), and 2-Nal. According to some embodiments, position 5 of the peptidomimetic is occupied by an amino acid selected from Gly (indan-2-yl), Phe(2-CF₃), 1-Nal, and 3-(4-Quinolyl)-alanine. Each possibility represents a separate embodiment of the invention. According to some embodiments, the amino acid at position 5 of the peptidomimetic is selected from Gly (indan-2-yl) and Phe(2-CF₃). Each possibility represents a separate embodiment of the invention.

According to some embodiments, position 5 of the peptidomimetic is occupied by an artificial AA comprising an aromatic moiety. According to some embodiments, the AA at position 5 comprising an aromatic moiety is selected from Gly (indan-2-yl), 1-Nal, Phe(3-5-di-F), and (Dmb)Gly.

According to some embodiments, position 5 of the peptidomimetic is occupied by an AA having a side-chain carboxyl moiety, wherein the side-chain carboxyl moiety is bonded to one or more amino acids such that the one or more bonded amino acids are not part of the peptidomimetic AA chain but create a branched chain. According to some embodiments, the side-chain carboxyl moiety is bonded to from 1 to 10 amino acids, including each value within the specified range. According to some particular embodiments, side-chain carboxyl moiety is bonded to from 1 to 4 amino acids, including each value within the specified range. One or more amino acids bonded to a side-chain carboxyl moiety are represented herein as a side-chain of the amino acid, enclosed in square brackets, i.e., “AA-[-short amino acid chain]”. According to some embodiments, the AA having a side-chain carboxyl moiety bonded to one or more amino acids is selected from L-isoAsp-[-GLY-GLY-GLY-3Abz], isoAad-[-1Nip-3Abz], isoAad-[-Val-ol], and L-isoGlu-[-Tyr(3-I)-DhPhe-Ser (Me)-DCit].

According to some embodiments, the peptidomimetic includes at position 5 the AA Gly (indan-2-yl). According to some embodiments, the peptidomimetic includes at position 5 the AA Phe(3-5-di-F). According to some alternative embodiments, the peptidomimetic includes at position 5 the AA 1-Nal.

### Combinations of 2 AA in the Peptidomimetic

According to some embodiments, the peptidomimetic comprises Gly at position 1, and an artificial AA which is alanine with at least a cyclic substituent, at position 2. According to some embodiments, the alanine with at least a cyclic substituent at position 2 is selected from Phe(3-5-di-F), beta-(2-furyl)-Ala, Cha, and Tza. According to some embodiments, the AA at position 2 is Phe(3-5-di-F). According to some further embodiments, the AA at position 2 is beta-(2-furyl)-Ala.

According to some embodiments, the peptidomimetic comprises Gly at position 1 and D-Glu at position 3.

According to some embodiments, the peptidomimetic comprises Gly at position 1 and an artificial AA which is alanine with at least a cyclic substituent, at position 4. According to some embodiments, the alanine with at least a cyclic substituent at position 4 is selected from 2-Nal, Phe(3-4-Cl), Cha, Phe(3,4,5-F), 1-Nal, and Ala (9-Anth). According to some particular embodiments, the AA at position 4 is Phe(3-4-Cl).

According to some embodiments, the peptidomimetic comprises Gly at position 1 and an artificial AA which is alanine with at least a cyclic substituent, at position 5. According to some embodiments, the alanine with at least a cyclic substituent at position 5 is selected from Phe(3-5-di-F) and 1-Nal. According to some particular embodiments, the AA at position 5 is Phe(3-5-di-F).

According to some embodiments, the peptidomimetic comprises D-Ala at position 1 and an artificial AA which is alanine with at least a cyclic substituent, at position 2. According to some embodiments, the alanine with at least a cyclic substituent at position 2 is selected from beta-(2-furyl)-Ala, Phe(3-5-di-F), Phe(3-OH), Pal(3), and Phe(3-OEt).

According to some embodiments, the peptidomimetic comprises D-Ala at position 1 and D-Glu at position 3.

According to some embodiments, the peptidomimetic comprises D-Ala at position 1 and artificial AA which is alanine with at least a cyclic substituent, at position 4. According to some embodiments, the alanine with at least a cyclic substituent at position 4 is selected from Phe(3-4-Cl), Phe(2-OCF₃), Phe(2-CN), and Dip.

According to some embodiments, the peptidomimetic comprises D-Ala at position 1 and Gly (indan-2-yl) at position 5.

According to some embodiments, the peptidomimetic comprises D-Pal(4) at position 1 and a non-DNA-encoded AA which is an alanine with at least a cyclic substituent at position 2. According to some embodiments, the alanine with at least a cyclic substituent at position 2 is selected from Tza, Pal(3), Phe(3-F), Phe(3-OMe), Phe(3-OH), Phe(3-Me), and Cha. According to some particular embodiments the AA at position 2 is Tza.

According to some embodiments, the peptidomimetic comprises D-Pal(4) at position 1 and D-Glu at position 3.

According to some embodiments, the peptidomimetic comprises D-Pal(4) at position 1 and Tyr(3-5-di-Iodo) at position 4.

According to some embodiments, the peptidomimetic comprises D-Pal(4) at position 1 and Gly (indan-2-yl) at position 5.

According to some embodiments, the peptidomimetic comprises D-aThr at position 1 and Tza at position 2.

According to some embodiments, the peptidomimetic comprises D-aThr at position 1 and D-Glu at position 3.

According to some embodiments, the peptidomimetic comprises D-aThr at position 1 and D-Abu(tetrazol-5-yl) at position 3.

According to some embodiments, the peptidomimetic comprises D-aThr at position 1 and Gly (indan-2-yl) at position 5.

According to some embodiments, the peptidomimetic comprises Tza at position 2 and D-Glu at position 3.

According to some embodiments, the peptidomimetic comprises Tza at position 2 and an artificial AA which is a tyrosine-derivative at position 4. According to some embodiments, the tyrosine-derivative at position 4 is selected from Tyr(3-5-di-Iodo), Tyr(3-5-di-Bromo), Tyr(3-NO₂), and Tyr(3-5-di-Cl). According to some particular embodiments, the tyrosine-derivative at position 4 is Tyr(3-5-di-Iodo).

According to some embodiments, the peptidomimetic comprises Tza at position 2 and an artificial AA which is a phenylalanine-derivative at position 4. According to some embodiments, the phenylalanine-derivative at position 4 is selected from Phe(3-4-Cl), Phe(2-NO₂), Phe(4-guanidino), Phe(3-4-diOMe), Phe(3-4-5-F), and Phe(4-NH₂).

According to some embodiments, the peptidomimetic comprises Tza at position 2 and Gly (indan-2-yl) at position 5.

According to some embodiments, the peptidomimetic comprises Phe(3-5-di-F) at position 2 and D-Glu at position 3.

According to some embodiments, the peptidomimetic comprises Phe(3-5-di-F) at position 2 and an artificial AA which is an alanine with at least a cyclic substituent, at position 4. According to some embodiments the alanine with at least a cyclic substituent, at position 4 is selected from 2-Nal, Cha, Ala (9-Anth), Phe(3-4-Cl), Phe(3,4,5-F), and Dip.

According to some embodiments, the peptidomimetic comprises Phe(3-5-di-F) at position 2 and Gly (indan-2-yl) at position 5.

According to some embodiments, the peptidomimetic comprises beta-(2-furyl)-Ala at position 2 and D-Glu at position 3.

According to some embodiments, the peptidomimetic comprises beta-(2-furyl)-Ala at position 2 and Phe(3-4-Cl) at position 4.

According to some embodiments, the peptidomimetic comprises Pal(3) at position 2 and D-Glu at position 3.

According to some embodiments, the peptidomimetic comprises Pal(3) at position 2 and Tyr(3-5-di-Iodo) at position 4.

According to some embodiments, the peptidomimetic comprises Pal(3) at position 2 and Gly (indan-2-yl) at position 5.

According to some embodiments, the peptidomimetic comprises a phenylalanine-derivative at position 2, selected from Phe(3-di-F), Phe(3-OH), Phe(3-Me), and Phe(3-OMe), and D-Glu at position 3.

According to some embodiments, the peptidomimetic comprises a phenylalanine-derivative at position 2, selected from Phe(3-OH), Phe(3-Me), Phe(3-OMe), Phe(3-OEt), and Tyr(3-5-di-Iodo) at position 4.

According to some embodiments, the peptidomimetic comprises a phenylalanine-derivative at position 2, selected from Phe(3-OH), Phe(3-Me), Phe(3-OMe), Phe(3-OEt), and Gly (indan-2-yl) at position 5.

According to some embodiments, the peptidomimetic comprises D-Glu at position 3 and a phenylalanine-derivative selected from Phe(3-4-Cl), Phe(2-NO₂), 4-carboxyPhe, L-Phe(2-OCF₃), Phe(2-CN), Phe(4-guanidino), and Phe(3-4-5-F) at position 4. According to some embodiments the phenylalanine-derivative is Phe(3-4-Cl).

According to some embodiments, the peptidomimetic comprises D-Glu at position 3 and a tyrosine derivative selected from Tyr(3-5-di-Iodo), Tyr(3-NO₂), and Tyr(3-5-di-Bromo). According to some particular embodiments, the tyrosine derivative is Tyr(3-5-di-Iodo).

According to some embodiments, the peptidomimetic comprises D-Glu at position 3 and an alanine derivative with at least a cyclic substituent, selected from 2-Nal, Cha, Ala (9-Anth), 1-Nal, and Dip.

According to some embodiments, the peptidomimetic comprises D-Glu at position 3 and

Phe(3-5-di-F) at position 5.

According to some embodiments, the peptidomimetic comprises D-Glu at position 3 and Gly (indan-2-yl) at position 5.

According to some embodiments, the peptidomimetic comprises Tyr(3-5-di-Iodo) at position 4 and Gly (indan-2-yl) at position 5.

According to some embodiments, the peptidomimetic comprises Tyr(3-5-di-Iodo) at position 3 and Gly (indan-2-yl) at position 5.

According to some embodiments, the peptidomimetic comprises an alanine with at least a cyclic substituent at position 4, selected from 2-Nal, Cha, and Phe(3-5-di-F) at position 5.

### Combinations of 3 or More AA in the Peptidomimetic

According to some embodiments, the peptidomimetic is according to Formula No. 2, which comprises Gly at position 1, D-Glu at position 3, and Phe(3-5-di-F) at position 5, herein: Gly-X-D-Glu-Y-Phe(3-5-di-F). According to some embodiments, X of Formula No. 2 is Phe(3-5-di-F). According to some further embodiments, X of Formula No. 2 is beta-(2-furyl)-Ala. According to some further embodiments, Y of Formula No. 2 is Phe(3-4-Cl). According to some further embodiments, Y of Formula No. 2 is Cha. According to some further embodiments, Y of Formula No. 2 is 2-Nal.

According to some embodiments, the peptidomimetic is according to Formula No. 3, which comprises D-Pal(4) at position 1, D-Glu at position 3, and Gly (indan-2-yl) at position 5 (herein: D-Pal(4)-X-D-Glu-Y-Gly (indan-2-yl)). According to some embodiments, X of Formula No. 3 is Tyr(3-5-di-Iodo). According to some specific embodiments, X of Formula No. 3 is Pal(3). According to other embodiments, Y of Formula No. 3 is selected from Tyr(3-5-di-Iodo) and 5-Bromo-2-fluoro-phenylalanine.

According to some embodiments, the peptidomimetic is according to Formula No. 4, which comprises D-Ala at position 1, D-Glu at position 3, and Gly (indan-2-yl) at position 5 (herein: D-Ala-X-D-Glu-Y-Gly (indan-2-yl)). According to some particular embodiments, Y of Formula No. 4 is a phenylalanine derivative selected from Phe(3-OH) and Phe(3-5-di-F). According to other embodiments, Y of Formula No. 4 is selected from Tyr(3-5-di-Iodo) and 5-Bromo-2-fluoro-phenylalanine.

According to some embodiments, the peptidomimetic is according to Formula No. 5, which comprises D-Pal(4) at position 1, D-Glu at position 3, and Tyr(3-5-di-Iodo) at position 4 (herein: D-Pal(4)-X-D-Glu-Y-Tyr(3-5-di-Iodo)).

According to some embodiments, the peptidomimetic is according to Formula No. 6, which comprises Tza at position 2, D-Glu at position 3, and Gly (indan-2-yl) at position 5 (herein: X-Tza-D-Glu-Y-Gly (indan-2-yl)). According to some particular embodiments, X of Formula No. 6 is D-aThr. According to some particular embodiments, Y of Formula No. 6 is Tyr(3-5-di-Iodo).

According to some embodiments, the peptidomimetic is according to Formula No. 7, which comprises Phe(3-5-di-F) at position 2 and position 5, and D-Glu at position 3 (herein: X-Phe(3-5-di-F)-D-Glu-Y-Phe(3-5-di-F)).

According to some embodiments, the peptidomimetic is according to Formula No. 8, which comprises D-Glu at position 3, Tyr(3-5-di-Iodo) at position 4, and Gly (indan-2-yl) at position 5 (herein: X-Y-D-Glu-Tyr(3-5-di-Iodo)-Gly (indan-2-yl)).

According to some embodiments, the peptidomimetic is according to Formula No. 9, which comprises D-aThr at position 1, D-Glu at position 3, and Gly (indan-2-yl) at position 5 (herein: D-aThr-X-D-Glu-Y-Gly (indan-2-yl)). According to some embodiments, X of Formula No. 9 is Tza. According to some specific embodiments, X of Formula No. 9 is Pal(3).

According to some embodiments, the peptidomimetic is according to Formula No. 10, which comprises Pal(3) at position 2, D-Glu at position 3, and Gly (indan-2-yl) at position 5 (herein: X-Pal(3)-(D-Glu)-Y-Gly (indan-2-yl)).

According to some embodiments, the peptidomimetic is according to Formula No. 1, which comprises D-Abu(tetrazol-5-yl) at position 3, Tyr(3-5-di-Iodo) at position 4, and Gly (indan-2-yl) at position 5 (herein: X-Y-D-Abu(tetrazol-5-yl)-Tyr(3-5-di-Iodo)-Gly (indan-2-yl)).

According to some embodiments, the peptidomimetic is according to a formula selected from Formulas 1 to 10, as set out below:

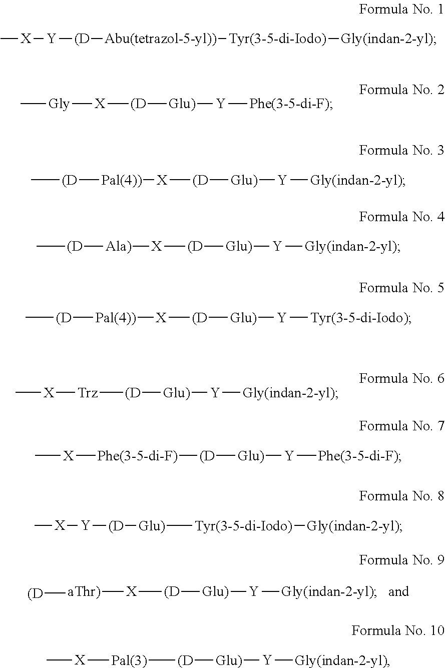

wherein X and Y of formulas 1-10 each as defined below.

According to some embodiments, X and/or Y of formulas 1-10 are each independently selected from the group consisting of: D-Alanine (D-Ala), D-Allothreonine (D-aThr), Allothreonine (aThr), D-Diaminopimelic (D-Dap), D-Norleucine (D-Nle), R-2-aminohepatonic acid (D-Nle(Me)), D-Cyclopropylglycine (D-Gly(cPr)), D-4-pyridyl-alanine (D-Pal(4)), Cyclopropylglycine (Gly(cPr)), D-Glutamine (D-Gln), D-Serine (D-Ser), D-homophenylalanine (D-hPhe), D-2-alpha-Aminobutyric-acid (D-Abu), L-2-pyridyl-alanine (Pal(2)), L-3-pyridyl-alanine (Pal(3)), Beta-(2-thienyl)-Ala (2Thi), 1-Aminocyclopropane-1-carboxylic acid (Ac3c), Beta-Cyclobutyl-L-Alanine (Ala(cBu)), Cyclohexylalanine (Cha), 6-hydroxynorleucine (L-Nle(6-OH)), 3-fluoro-phenylalanine (Phe(3-F)), 3-methyl-phenylalanine (Phe(3-Me)), 3-hydroxy-phenylalanine (Phe(3-OH)), 3-methoxy-phenylalanine (Phe(3-OMe)), 3-ethoxy-phenylalanine (Phe(3-OEt)), 2-methoxy-phenylalanine (Phe(2-OMe)), Beta-(4-thiazolyl)-Ala (Tza), Homoleucine (hLeu), L-3-thienylalanine (3Thi), D-2-Furylalanine (beta-(2-furyl)-D-Ala), L-2-Furylalanine (beta-(2-furyl)-Ala), 3-5-difluoro-phenylalanine (Phe(3-5-di-F)), D-Glutamic acid (D-Glu), gamma-Carboxy-D-glutamic-acid (D-Gla), D-2-alpha-Aminobutyric-acid (tetrazol-5-yl) (D-Abu(tetrazol-5-yl)), D-Cys(-Methyl-2H-tetrazole), D-2-aminoadipic acid (D-Aad), D-Asp-[1-nipecotic acid] (D-Asp-[-1Nip]), D-Cysteic Acid (D-Cys (O₃H)), 3,5-Di-Iodo-tyrosine (Tyr(3-5-di-Iodo)), (2-naphthyl)-alanine (2-Nal), (1-naphthyl)-alanine (1-Nal), 4-carboxyphenylalanine (4-carboxyPhe), 5-methoxy-tryptophan, L-9-Anthrylalanine (Ala (9-Anth)), 3-3-diphenyl-L-alanine (Dip), 3-4-dimethoxy-phenylalanine (Phe(3-4-diOMe)), 3,5-dichloro-tyrosine (Tyr(3-5-di-Cl), L-isoAsp-[3-Aminobenzoic acid] (L-isoAsp-[-3Abz]), 2-(trifluoromethoxy)-phenylalanine (Phe(2-OCF₃)), L-2-Cyanophenylalanine (Phe(2-CN)), 2-nitro-phenylalanine (Phe(2-NO₂)), 4-Guanidinophenylalanine (Phe(4-guanidino)), 3-4-dichloro-phenylalanine (Phe(3-4-Cl)), (S)-2-Methoxy-phenylglycine (Phg(2-OMe)), 4-Amino-phenylalanine (Phe(4-NH₂)), 3-iodo-tyrosine (Tyr(3-Iodo)), 5-Bromo-2-fluoro-phenylalanine, pentafluoro-phenylalanine (Phe(F₅)), 7-Azatryptophan (L-(7-azaTrp)), 3-5-di-Bromo-Tyrosine (Tyr(3-5-di-Bromo)), 2-trifluoromethyl-phenylalanine (Phe(2-CF₃)), 3-(4-Quinolyl)-alanine, 3-Nitro-tyrosine (Tyr(3-NO₂)), 3-Bromo-5-fluoro-phenylalanine, L-2-indanylglycine (Gly (indan-2-yl)), isoAad-[-Val-ol], isoAad-[-1Nip-3 Abz], L-Asp-[-GLY-GLY-GLY-3Abz], N-(2-4-dimethoxybenzyl)-Gly) (Dmb (Gly)), 5-phenyl-L-norvaline (Nva(Ph)), Homoserine (Hse), L-thyroxine, 2-Amino-3-(7-methoxy-4-coumaryl) propionic acid (beta-(7-methoxy-coumarin-4-yl)-Ala), 3,4,5-Trifluorophenylalanine (Phe(3,4,5-F)), 4-guanidino-phenylalanine (Phe(4-guanidino)), 3-methyl-tyrosine (Tyr(3-Me)), 3,5-Di-Bromophenylalanine (Phe(3-5-di-Br)), N-methylated 3-iodo-tyrosine (Nme-Tyr(3-Iodo)), Kynurenine (Asp(Ph(2-NH₂))), 2-amino-2-indancarboxylic-acid (Aic), S-xanthyl-L-cysteine (Cys(Xan)), Cyclopropylalanine (Ala (cPr)), Sarcosine (Nme-Gly), 4-phenyl-L-phenylalanine (Bip), 4-Benzoylphenylalanine (Bpa), 1-Benzyl-D-histidine (D-His(1-Bn)), Homotyrosine (hTyr), D-Serine-acetate (D-Ser (Ac)), 3,5-dimethyl-tyrosine (Tyr(3-5-di-Me)), L-isoGlu-[-Tyr(3-I)-DhPhe-Ser (Me)-DCit], L-2-Amino-3-guanidinopropionic-acid (Ala (guanidino)), 2-Amino-3,3-bis(4-fluorophenyl) propanoic acid, 3-Nitro-D-phenylalanine (D-Phe(3-NO₂)), beta-hydroxyphenylalanine (Phe (bR-OH)), Gly, Nle, Met, Ala, and Trp.

According to some embodiments, the present invention provides a head-to-tail cyclic penta-peptidomimetic that inhibits the interaction between an interleukin 17 (IL-17) and an IL-17 receptor (IL-17R), wherein:
the amino acid or at position 1, is selected from the group consisting of D-Ala, D-aThr, D-Pal(4), D-Gln, D-Abu, D-Gly(cPr), D-Nle(Me), Gly, D-Ser, D-Nle, beta-(2-furyl)-D-Ala, D-hPhe, D-Dap, and Nme-Gly, or the non-amino acid moiety at position 1 is selected from propylamine and cysteamine;
the amino acid at position 2 is selected from the group consisting of Pal(3), Phe(3-OEt), Phe(3-OMe), Phe(3-5-di-F), Nle, beta-(2-furyl)-Ala, hLeu, Tza, 2Thi, 3Thi, Met, L-Nle(6-OH), Cha, Pal(2), Ac3c, Phe(3-F), Ala(cBu), Phe(3-Me), aThr, Phe(3-OH), Gly(cPr), Ala (cPr), Ala, and beta-(2-furyl)-D-Ala, or the non-amino acid moiety at position 2 is selected from 4-Mercaptophenylacetic Acid (MPAA) and (1,3-Dimethylbenzene)-3-sulfanylpropanoic acid;
the amino acid at position 3 is selected from the group consisting of D-Abu(tetrazol-5-yl), D-Cys(-Methyl-2H-tetrazole)), D-Glu, D-Cys (O₃H), D-Aad, D-Ser (Ac), D-Phe(3-NO₂), D-Asp-[-1Nip], and D-Gla;
the amino acid at position 4 is selected from the group consisting of Tyr(3-5-di-Iodo, 5-Bromo-2-fluoro-phenylalanine, Phe(2-OCF₃), Dip, Phe(2-OMe), Phe (bR-OH), Phe(3-5-di-Br), 2-Nal, Cha, Tyr(3-NO₂), Phe(3-4-Cl), Ala (9-Anth), Phe(3,4,5-F), Trp, 1-Nal, Phe(2-NO₂), Phe(4-guanidino), Tyr(3-5-di-Bromo), L-isoAsp-[-3Abz], 5-methoxy-tryptophan, 4-CarboxyPhe, Phe(2-CN), Tyr(3-5-di-Cl), Phe(3-4-diOMe), Phe(4-NH₂), L-(7-azaTrp), Cys(Xan), Tyr(3-5-di-Me), Phe(F₅), Tyr(3-Iodo), L-thyroxine, Phg(2-OMe), 3-Bromo-5-fluoro-phenylalanine, beta-(7-methoxy-coumarin-4-yl)-Ala, Tyr(3-Me), Bip, Nme-Tyr(3-Iodo), Bpa, D-His(1-Bn), hTyr, 2-Amino-3,3-bis(4-fluorophenyl) propanoic acid, Phe(3-4-5-F), and Nva(Ph); and the amino acid at position 5 is selected from the group consisting of Gly (indan-2-yl), Phe(2-CF₃), 1-Nal, 3-(4-Quinolyl)-alanine, L-(7-azaTrp), Phe(2-OCF₃), 2-Nal, Phe(3-5-di-F), Hse, L-Asp-[-GLY-GLY-GLY-3Abz], isoAad-[-1Nip-3 Abz], isoAad-[-Val-ol], L-isoGlu-[-Tyr(3-I)-DhPhe-Ser (Me)-DCit], Trp, Asp(Ph(2-NH₂)), Aic, Ala, Phg(2-OMe), (Dmb)Gly, and Ala (guanidino).

According to some embodiments, the at least one cyclic penta-peptidomimetic is selected from the group consisting of Compound Nos. 1 to 188, or a pharmaceutically acceptable salt thereof.

According to some embodiments, the peptidomimetic does not bind Leu97A and/or Leu97B of the IL-17A protein. According to some embodiments, the peptidomimetic according to the invention does not bind the backbone of Leu97A and/or Leu97B of the IL-17 protein, via a bis amide of the peptidomimetic.

### Potency

According to some embodiments, the peptidomimetic according to the invention is characterized by an IL-17 inhibitory activity, defined as p(IC₅₀) (i.e., inhibition of IL-17-IL-17R interaction, measured as indicated below) of 4 or above. According to some further embodiments, the peptidomimetic is characterized by an IL-17 inhibitory activity of p(IC₅₀)=4.5 or above, p(IC₅₀)=5 or above, p(IC₅₀)=5.5 or above, p(IC₅₀)=6 or above, p(IC₅₀)=6.5 or above, p(IC₅₀)=7 or above, or p(IC₅₀)=7.5 or above. Each possibility represents a separate embodiment of the invention. According to some embodiments, the peptidomimetic is characterized by an IL-17 inhibitory activity of between 4 and 8.5, including each value within the specified range. According to some embodiments, the peptidomimetic is characterized by an IL-17 inhibitory activity of between 5.5 and 8, including each value within the specified range. According to some embodiments, the peptidomimetic is characterized by an IL-17 inhibitory activity of between 6.5 and 8, including each value within the specified range. According to some embodiments, the peptidomimetic is characterized by an IL-17 inhibitory activity of between 7 and 8, including each value within the specified range. The inhibitory activity is measured for at least one of IL-17A-IL-17R interaction, IL-17F-IL-17R interaction, and IL-17AF-IL-17R interaction. The p(IC₅₀) value of a peptidomimetic, as referred to herein, is as determined by the results of at least one of ELISA, AlphaLISA, Microscale Thermophoresis, a combination thereof, or by any methods known in the art to determine inhibition or the dissociation constant (K_{d}) of a biological interaction. According to some embodiments, the p(IC₅₀) is determined by a single method. According to some embodiments, the p(IC₅₀) is determined by AlphaLisa. According to some embodiments, the p(IC₅₀) is the average p(IC₅₀) as determined by AlphaLisa and ELISA. According to some embodiments, the p(IC₅₀), as determined by AlphaLisa, is at least 6.5. According to some embodiments, the p(IC₅₀), as determined by AlphaLisa, is at least 7.0. According to some embodiments, the present invention provides a cyclic penta-peptidomimetic that inhibits the interaction between IL-17 and IL-17R, wherein:
the amino acid at position 1, counted from the N-terminus of the pentapeptide before cyclization, is selected from the group consisting of D-Ala, D-aThr, D-Pal(4), D-Gln, D-Abu, D-Gly(cPr), and D-Nle(Me);
the amino acid at position 2 is selected from the group consisting of Pal(3), Phe(3-OEt), Phe(3-OMe), and Phe(3-5-di-F);
the amino acid at position 3 is selected from the group consisting of D-Abu(tetrazol-5-yl), D-Cys(-Methyl-2H-tetrazole)), and D-Glu;
the amino acid at position 4 is selected from the group consisting of Tyr(3-5-di-Iodo), 5-Bromo-2-fluoro-phenylalanine, Phe(2-OCF₃), Dip, Phe(2-OMe), Phe (bR-OH), and Phe(3-5-di-Br); and
the amino acid at position 5 is selected from the group consisting of Gly (indan-2-yl), Phe(2-CF₃), 1-Nal, 3-(4-Quinolyl)-alanine, L-(7-azaTrp), Phe(2-OCF₃), and 2-Nal.
wherein the p(IC₅₀), as determined by AlphaLisa, is at least 6.5. Each possibility represents a separate embodiment.

According to some embodiments, the p(IC₅₀), as determined by AlphaLisa, is at least 6.7 and the peptidomimetic is selected from Compound Nos. 134, 127, 123, 78, 157, 178, 169, 153, 140, 136, 125, 121, 152, 156, 146, 174, 177, 176, 92, 135, 155, 163, 150, 154, 173, 93, 171, 117, 168, 151, 101, 137, 114, 175, 180, 183, 184, 185, and 30.

According to some embodiments, the p(IC₅₀), as determined by AlphaLisa, is at least 7.0 and the peptidomimetic is selected from Compound Nos. 134, 127, 123, 78, 157, 178, 169, 153, 140, 136, 125, 121, 152, 156, 146, 174, 177, 176, 92, 135, 155, 163, 150, 154, 173, 93, 171, 117, 168, 101, 137, 114, 175, 180, 183, 184, 185, and 30.

According to some embodiments, the p(IC₅₀), as determined by the average of the AlphaLisa and ELISA p(IC₅₀) values, is at least 7. According to some embodiments, the p(IC₅₀), as determined by the average of the AlphaLisa and ELISA p(IC₅₀) values, is between 7 and 7.8. According to some embodiments, the IC₅₀ is between about 10 and 100 nM. According to some embodiments, the p(IC₅₀), as determined by the average of the AlphaLisa and ELISA p(IC₅₀) values, is between 7 and 7.8 and the amino acid at position 1 is a D-amino acid. According to some embodiments, the p(IC₅₀), as determined by the average of the AlphaLisa and ELISA p(IC₅₀) values, is between 7 and 7.8 and the amino acid at position 1 is selected from the group consisting of D-Ala, D-aThr, D-Pal(4), D-Gln, D-Abu, D-Gly(cPr), and D-Nle(Me). According to some embodiments, the p(IC₅₀), as determined by the average of the AlphaLisa and ELISA p(IC₅₀) values, is between 7 and 7.8 and the amino acid at position 1 is D-Ala.

According to some embodiments, the p(IC₅₀), as determined by the average of the AlphaLisa and ELISA p(IC₅₀) values, is between 7 and 7.8 and the amino acid at position 2 is a 6-member aromatic ring with a hydrogen bond accepting moiety as either a part of the aromatic ring or directly attached to it. According to some embodiments, the p(IC₅₀), as determined by the average of the AlphaLisa and ELISA p(IC₅₀) values, is between 7 and 7.8 and the amino acid at position 2 is selected from the group consisting of Pal(3), Phe(3-OEt), and Phe(3-OMe). According to some embodiments, the p(IC₅₀), as determined by the average of the AlphaLisa and ELISA p(IC₅₀) values, is between 7 and 7.8 and the amino acid at position 2 is Pal(3).

According to some embodiments, the p(IC₅₀), as determined by the average of the AlphaLisa and ELISA p(IC₅₀) values, is between 7 and 7.8 and the amino acid at position 3 position 3 is composed of negatively charged or partially negatively charged residues of D chirality. According to some embodiments, the p(IC₅₀), as determined by the average of the AlphaLisa and ELISA p(IC₅₀) values, is between 7 and 7.8 and the amino acid at position 3 is selected from the group consisting of D-Abu(tetrazol-5-yl), D-Cys(-Methyl-2H-tetrazole)), and D-Glu. According to some embodiments, the p(IC₅₀), as determined by the average of the AlphaLisa and ELISA p(IC₅₀) values, is between 7 and 7.8 and the amino acid at position 3 is D-Abu(tetrazol-5-yl).

According to some embodiments, the p(IC₅₀), as determined by the average of the AlphaLisa and ELISA p(IC₅₀) values, is between 7 and 7.8 and the amino acid at position 4 comprises modified phenylalanine residues of L chirality. According to further embodiments, the modified phenylalanine residues comprise volume-filling moieties. According to some embodiments, the p(IC₅₀), as determined by the average of the AlphaLisa and ELISA p(IC₅₀) values, is between 7 and 7.8 and the amino acid at position 4 is selected from the group consisting of 3,5-di-Iodo-L-tyrosine (Tyr(3-5-di-Iodo), 5-Bromo-2-fluoro-L-phenylalanine, 2-(trifluoromethoxy)-L-phenylalanine (L-Phe(2-OCF₃), 3-3-diphenyl-L-alanine (Dip), 2-Methoxy-L-Phenylalanine (Phe(2-OMe), and 3,5-diBromophenylalanine (Phe(3-5-di-Br).

According to some embodiments, the p(IC₅₀), as determined by the average of the AlphaLisa and ELISA p(IC₅₀) values, is between 7 and 7.8 and the amino acid at position 5 comprises a single 6-membered aromatic ring or a double aromatic ring. According to further embodiments, the amino acid at position 5 comprises homoaromatic or heteroaromatic rings. According to some embodiments, the p(IC₅₀), as determined by the average of the AlphaLisa and ELISA p(IC₅₀) values, is between 7 and 7.8 and the amino acid at position 5 is selected from the group consisting Gly (indan-2-yl), Phe(2-CF₃), 1-Nal, 3-(4-Quinolyl)-alanine, L-(7-azaTrp), Phe(2-OCF₃), and 2-Nal. According to some embodiments, the p(IC₅₀), as determined by the average of the AlphaLisa and ELISA p(IC₅₀) values, is between 7 and 7.8 and the amino acid at position 5 is Gly (indan-2-yl).

According to some embodiments, the p(IC₅₀), as determined by the average of the AlphaLisa and ELISA p(IC₅₀) values, is between 7 and 7.8 and the peptidomimetic is selected from Compound Nos. 134, 127,123, 78, 157, 178, 169, 153, 140, 136, 125, 121, 152, 156, 146, 174, 177, 176, 92, 135, 155, 163, 150, 154, 173, 93, 171, 117, 168, 151, 101, 137, 114, 175,180, 183, and 184.

According to some embodiments, the peptidomimetic comprises an amino acid selected from D-Ala, D-aThr, and D-Pal(4) at position 1; the amino acid Pal(3) at position 2; an amino acid selected from D-Abu(tetrazol-5-yl) and D-Glu at position 3; an amino acid selected from Tyr(3-5-di-Iodo), 5-Bromo-2-fluoro-phenylalanine, and Phe(2-OCF₃) at position 4; and an amino acid selected from Gly (indan-2-yl), Phe(2-CF₃), 1-Nal, and 3-(4-Quinolyl)-alanine at position 5. According to other embodiments, the amino acid at position 3 is D-Abu(tetrazol-5-yl). Each possibility represents a separate embodiment of the invention.

According to some embodiments, the peptidomimetic is selected from Compound Nos. 78, 123, 125, 127, 134, 136, 137, 140, 146, 153, 154, 155, 168, 175, 176, 177, 109, 41, 64, and 47. According to some embodiments, the peptidomimetic is selected from Compound Nos. 78, 123, 125, 127, 134, 136, 137, 140, 146, 153, 154, 155, 168, 175, 176, and 177.

The original peptidomimetics as disclosed herein can be modified by the substitution of one or more residues at different, possibly selective, sites within the peptide chain, if not otherwise stated. Such substitutions may be of a conservative nature, for example, where one amino acid is replaced by an amino acid having similar structure and characteristics, such as where a hydrophobic amino acid is replaced by another hydrophobic amino acid. Even more conservative would be replacement of amino acids of the same or similar size and chemical nature, such as where leucine is replaced by isoleucine. In studies of sequence variations in families of naturally occurring homologous proteins, certain amino acid substitutions are more often tolerated than others, and these often show a correlation with similarities in size, charge, polarity, and hydrophobicity between the original amino acid and its replacement, and such is the basis for defining “conservative substitutions”.

Conservative substitutions are herein defined as exchanges within one of the following five groups: Group 1-small aliphatic, nonpolar or slightly polar residues (Ala, Ser, Thr, Pro, Gly); Group 2-polar, negatively charged residues and their amides (Asp, Asn, Glu, Gln); Group 3-polar, positively charged residues (His, Arg, Lys); Group 4-large, aliphatic, nonpolar residues (Met, Leu, Ile, Val, Cys); and Group 5-large, aromatic residues (Phe, Tyr, Trp).

Less conservative substitutions might involve the replacement of one amino acid by another that has similar characteristics but is somewhat different in size, such as replacement of an alanine by an isoleucine residue. Highly non-conservative replacements might involve substituting an acidic amino acid for one that is polar, or even for one that is basic in character. Such “radical” substitutions cannot, however, be dismissed as potentially ineffective since chemical effects are not totally predictable and radical substitutions might well give rise to serendipitous effects not otherwise predictable from simple chemical principles.

### Pharmaceutical and Cosmetic Compositions and Uses Thereof

According to some further embodiments, the present invention provides a pharmaceutical composition comprising a compound and/or salt of one of the embodiments of the peptidomimetics as described herein above, and at least pharmaceutically acceptable carrier, diluent and/or excipient.

Compounds of the present invention are potent inhibitors of the interaction of IL-17A, IL-17F, and IL-17AF with IL-17 receptor, and upon administration to a patient in need thereof, may provide therapeutic benefits while avoiding certain problems associated with inhibitory antibodies for IL-17. As such, compounds of the present invention are believed to be useful for the treatment, prevention, and/or improving symptoms of diseases or conditions in which excessive IL-17A mediated signaling plays a role, including relief of certain immunologically- or inflammatory-mediated symptoms. Such conditions include, for example, psoriasis, psoriatic arthritis, rheumatoid arthritis, spondyloarthritis, multiple sclerosis, axial spondyloarthritis, ankylosing spondylitis, hidradenitis suppurativa, systemic lupus erythematosus, palmoplantar pustulosis (PPP), atopic dermatitis, asthma, inflammatory bowel disease (IBD), COPD, age-associated skin condition, and allograft rejection.

According to some particular embodiments of the invention, there is provided a pharmaceutical composition for treating psoriasis, comprising at least one peptidomimetic according to the invention as described hereinabove, or a pharmaceutically acceptable salt thereof, and one or more pharmaceutically acceptable excipients, carriers, or diluents. In a further embodiment the invention provides a pharmaceutical composition for treating rheumatoid arthritis, comprising at least one peptidomimetic according to the invention as described hereinabove, or a pharmaceutically acceptable salt thereof, and one or more pharmaceutically acceptable excipients, carriers, or diluents. In another embodiment the invention provides a pharmaceutical composition for treating multiple sclerosis, comprising at least one peptidomimetic according to the invention as described hereinabove, or a pharmaceutically acceptable salt thereof, and one or more pharmaceutically acceptable excipients, carriers, or diluents.

The peptidomimetics and/or compositions disclosed herein may also be used in combination with one or more other active ingredients. In certain embodiments, the compounds may be administered in combination, or sequentially, with another therapeutic agent. Such other therapeutic agents include those known for treatment, prevention, or amelioration one or more symptoms associated with, for example, inflammation or autoimmune disease.

In certain further embodiments, combinations of 2 or more of the peptidomimetics disclosed herein may be used in treatment of a subject in need thereof. The combination may include formulating a composition comprising 2 or more different peptidomimetics.

According to further embodiments, the present invention provides a method for treating a patient suffering from an IL-17 associated disease, condition, or disorder, selected from the group consisting of psoriasis, psoriatic arthritis, rheumatoid arthritis, spondyloarthritis, multiple sclerosis, axial spondyloarthritis, ankylosing spondylitis, hidradenitis suppurativa, systemic lupus erythematosus, palmoplantar pustulosis (PPP), atopic dermatitis, asthma, COPD, age-associated skin condition, and/or allograft rejection, the method comprising administering to a patient in need thereof an effective amount of at least one peptidomimetic according to the invention as described hereinabove, or a pharmaceutically acceptable salt thereof.

According to some particular embodiments, the method of treating a patient suffering from a disease or condition associated with elevated levels of IL-17A (and/or IL-17F/IL-17AF) comprises a preliminary step of determining whether the patient has an elevated level of one or more IL-17-induced chemokine or effector, and, if the result is in the affirmative, administering to the patient an effective amount of at least one peptidomimetic according to the present invention, or of a composition comprising such a peptidomimetic, to treat the disease or condition. The measured IL-17A dependent chemokine or effector may be one or more of IL-8, Gro-alpha (i.e., Cxcl1 gene), IL-1A, TNF-alpha, IL-6, IL-22, S100a7a, CK-16, and IL-1beta.

Further, the present invention provides a method of treating psoriasis, comprising administering to a patient in need thereof an effective amount of at least one peptidomimetic according to the invention as described hereinabove, or a pharmaceutically acceptable salt thereof. Further, the present invention provides a method of treating rheumatoid arthritis, comprising administering to a patient in need thereof an effective amount of at least one peptidomimetic according to the invention as described hereinabove, or a pharmaceutically acceptable salt thereof.

Further, the present invention provides a method of treating multiple sclerosis, comprising administering to a patient in need thereof an effective amount of at least one peptidomimetic according to the invention as described hereinabove, or a pharmaceutically acceptable salt thereof. Further, the present invention provides a method of treating spondyloarthritis, comprising administering to a patient in need thereof an effective amount of at least one peptidomimetic according to the invention as described hereinabove, or a pharmaceutically acceptable salt thereof.

Any stage and severity of plaque psoriasis may be treated with the compositions of the present invention, including mild, moderate, and severe stages. According to some embodiments, the disease is moderate to severe plaque psoriasis. According to some embodiments, the disease is moderate plaque psoriasis. According to other embodiments, the disease is severe plaque psoriasis.

In specific embodiments, the compositions of the present invention are administered to a subject suffering from psoriasis, as part of a treatment regimen that includes additional therapies and treatment for psoriasis. The additional therapies and treatments may include but are not limited to topical corticosteroids, Acitretin, vitamin D analogs, methotrexate, and cyclosporine.

In human therapeutics, the physician will determine the dosage regimen that is most appropriate according to a preventive or curative treatment and according to the age, weight, stage of the disease and other factors specific to the subject to be treated. The amount of active ingredient in the pharmaceutical composition provided herein, which will be effective in the prevention or treatment of the IL-17 associated disease will vary with the nature and severity of the disease or condition, and the route by which the active ingredient is administered. The frequency and dosage will also vary according to factors specific for each subject depending on the specific therapy (e.g., therapeutic or prophylactic agents) administered, the severity of the disease or condition, the route of administration, as well as age, body, weight, response, and the past medical history of the subject. The active ingredient may be administered at once or may be divided into a number of smaller doses to be administered at intervals of time.

According to some particular embodiments, administration of the pharmaceutical composition is local, parenteral, or enteral. Each possibility represents a separate embodiment. According to some embodiments, administration of the pharmaceutical composition is topical, intravenous, or oral. According to some embodiments, administration of the pharmaceutical composition is topical. According to some embodiments, administration of the pharmaceutical composition is intravenous. According to some embodiments, administration of the pharmaceutical composition is oral. According to some embodiments, administration of the pharmaceutical composition is by injection, infusion, inhalation or as a nasal spray. According to some embodiments, administration of the pharmaceutical composition is intradermal or subcutaneous.

According to some embodiments, administration of the pharmaceutical composition is locally to the dermis. According to some embodiments, administration of the pharmaceutical composition is by electroporation, using methods and devices known in the art.

According to some embodiments, administration of the pharmaceutical composition is topical. According to some embodiments, administration of the pharmaceutical composition is intravenous. According to some embodiments, administration of the pharmaceutical composition is oral. Other administration routes known in the art are also contemplated.

In one embodiment, the present invention provides at least one peptidomimetic as described hereinabove, or a pharmaceutically acceptable salt thereof, for use in the treatment of psoriasis. In another particular embodiment the invention provides at least one peptidomimetic as described hereinabove, or a pharmaceutically acceptable salt thereof, for use in the treatment of rheumatoid arthritis. In another particular embodiment the invention provides at least one peptidomimetic as described hereinabove, or a pharmaceutically acceptable salt thereof, for use in treating multiple sclerosis. In another particular embodiment the invention provides at least one peptidomimetic as described hereinabove, or a pharmaceutically acceptable salt thereof, for use in treating spondyloarthritis. In another particular embodiment the invention provides at least one peptidomimetic as described hereinabove, or a pharmaceutically acceptable salt thereof, for use in treating a disease or disorder selected from the group consisting of psoriasis, psoriatic arthritis, rheumatoid arthritis, spondyloarthritis, multiple sclerosis, axial spondyloarthritis, ankylosing spondylitis, hidradenitis suppurativa, systemic lupus erythematosus, palmoplantar pustulosis (PPP), atopic dermatitis, asthma, IBD, COPD, age-associated skin condition, and/or allograft rejection.

In yet another embodiment, the present invention provides the use of at least one peptidomimetic as described hereinabove, or a pharmaceutically acceptable salt thereof, in the manufacture of a medicament for the treatment of psoriasis. In yet another embodiment, the present invention provides the use of at least one peptidomimetic as described hereinabove, or a pharmaceutically acceptable salt thereof, in the manufacture of a medicament for the treatment of rheumatoid arthritis. In yet another embodiment, the present invention provides the use of at least one peptidomimetic as described hereinabove, or a pharmaceutically acceptable salt thereof, in the manufacture of a medicament for the treatment of multiple sclerosis. In yet another embodiment, the present invention provides the use of at least one peptidomimetic as described hereinabove, or a pharmaceutically acceptable salt thereof, in the manufacture of a medicament for the treatment of spondyloarthritis. In yet another embodiment, the present invention provides the use of at least one peptidomimetic as described hereinabove, or a pharmaceutically acceptable salt thereof, in the manufacture of a medicament for the treatment of disease or disorder selected from the group consisting of psoriasis, psoriatic arthritis, rheumatoid arthritis, spondyloarthritis, multiple sclerosis, axial spondyloarthritis, ankylosing spondylitis, hidradenitis suppurativa, systemic lupus erythematosus, palmoplantar pustulosis (PPP), atopic dermatitis, asthma, IBD, COPD, age-associated skin condition, and/or allograft rejection.

According to some embodiments, the peptidomimetic is selected from the compounds (Comp.) presented in Table 1 and FIG. 1.

According to some embodiments, a cosmetic composition is provided, comprising at least one cyclic penta-peptidomimetic as disclosed herein above, and at least one cosmetically acceptable excipient, salt, or carrier.

The term ‘cosmetically acceptable’ as used herein refers to all carriers and/or excipients and/or diluents and/or salts conventionally used in cosmetic compositions and that do not abrogate the beneficial activity and properties of the peptide of the present invention.

According to other embodiments, a cosmetic composition is provided, comprising at least one cyclic penta-peptidomimetic as disclosed herein above, and at least one dermatologically acceptable excipient, salt, or carrier.

The term “dermatologically acceptable” as used herein refers to a carrier and/or an excipient and/or a diluent and/or a salt that does not cause significant irritation to the skin and that do not abrogate the beneficial activity and properties of the peptide of the present invention. Suitable dermatologically acceptable carriers within the scope of the present invention include, but are not limited to, a thickener, a filler, a moisturizer, an emulsifier, a humectant, a surfactant, a buffering or pH adjusting agent, a film forming agent, a foaming agent, an anti-foaming agent, a preservative, an anti-oxidant, a fragrance, a solvent, a propellant, a colorant, and a combination or mixture thereof. Each possibility represents a separate embodiment.

According to some embodiments, the cosmetic composition comprises at least two different cyclic penta-peptidomimetics.

According to some embodiments, the cosmetic composition comprises at least one additional ingredient. Ingredients that may be included in the compositions of the present invention are vitamins and vitamin derivatives such as, for example vitamin A, vitamin B, vitamin D, vitamin E, vitamin K and derivatives thereof including, for example, α tocopherol; and various plant extracts such as, for example, shea butter, Aloe vera, Aloe barbadensis, castor oil, Citrus limonium, Citrus paradisi, Citrus sinensis, Elaesis guineensis, etc. Additional ingredients that may be included in the compositions of the present invention are sunscreens and tanning agents. Sunscreens include those materials commonly employed to block UV light. Illustrative compounds are the derivatives of PABA, cinnamate and salicylate. For example, octyl methoxycinnamate and 2-hydroxy-4-methoxy benzophenone (also known as oxybenzone) can be used. Octyl methoxycinnamate and 2-hydroxy-4-methoxy benzophenone are also known as parsol MCX and benzophenone-3, respectively. Other ingredients may include coal tar, salicylic acid, ceramides, glycerin, hyaluronic acid, and retinoids.

According to some embodiments, the cosmetic composition is formulated for local administration. According to some embodiments, the local administration is topical administration. According to some embodiments, the local administration is intradermal administration.

According to some embodiments, the cosmetic composition may be formulated as an oil, a gel, a stick, a lotion, a cream, a milk, an aerosol, a spray, a foam, a mousse, an ointment, liquid drops, nebulized liquid, a liquid wash, an emulsion, a suspension, liposomes, an adhesive patch, and a powder. Each possibility represents a separated embodiment. According to some embodiments, the cosmetic composition is formulated as a lotion, a cream, an ointment, a paste, a gel, a hydrogel and the like, or as powder. Each possibility represents a separated embodiment. According to other embodiments, the cosmetic composition is formulated as a solution, mist, or spray.

According to some embodiments, the cosmetic composition is for use in the prevention, treatment, alleviation of a cosmetic condition, disorder or disease.

According to some embodiments, the cosmetic condition comprises a skin disorder or an appearance of the skin.

According to some embodiments, the cosmetic condition is selected from, but not limited to: acne (particularly mild to moderate, and including post-acne marks), excess oil production (including seborrhoea), enlarged pores, hyperpigmentation (including post-inflammatory hyperpigmentation), melasma, dark spots, sunspots and age spots, age signs and wrinkles (including fine lines and deep wrinkles), loss of skin elasticity, sagging and dull skin, sensitive and reactive skin (e.g., rosacea, or eczema), skin redness and irritation, rosacea-prone skin, eczema and atopic dermatitis marks, barrier dysfunction, dry and dehydrated skin, flaky, rough skin, tightness and discomfort skin, xerosis (severe dry skin), eye dark circles and puffy eyes, under-eye pigmentation, puffiness and swelling, fine lines and crepey skin. Each possibility represents a separate embodiment.

According to some embodiments, the cosmetic condition is associated with psoriasis. According to some embodiments, the cosmetic condition associated with psoriasis is selected from one or more of altered skin texture, skin discoloration, and flaky scalp.

According to some embodiments, a cosmetic composition is provided for use in the prevention, treatment, alleviation of a cosmetic condition, disorder or disease.

According to some embodiments, the present invention provides a method for preventing, treating, or alleviating a cosmetic condition or disorder, the method comprising administering an effective amount of a compound according to the present invention, or a cosmetically acceptable salt thereof. According to some embodiments, the present invention provides a method for preventing, treating, or alleviating a cosmetic condition or disorder, the method comprising administering an effective amount of a compound according to the present invention, or a dermatologically acceptable salt thereof.

According to some embodiments, the cosmetic condition comprises a skin disorder or an appearance of the skin. According to some embodiments, the cosmetic condition comprises a skin disorder or an appearance of the skin related to aging. According to some embodiments, the cosmetic condition is related to psoriasis. According to some embodiments, the cosmetic condition related to psoriasis is selected from skin texture, skin discoloration, and flaky scalp.

According to some embodiments, the cosmetic composition is administered by local administration. According to some embodiments, the local administration is topical administration.

According to some embodiments, the local administration is intradermal administration.

According to some embodiments, the cosmetic composition is administered as an oil, a gel, a stick, a lotion, a cream, a milk, an aerosol, a spray, a foam, a mousse, an ointment, liquid drops, nebulized liquid, a liquid wash, an emulsion, a suspension, liposomes, an adhesive patch, and a powder. Each possibility represents a separated embodiment. According to some embodiments, the cosmetic composition is administered as a lotion, a cream, an ointment, a paste, a gel, a hydrogel, and the like, or as a powder. Each possibility represents a separated embodiment.

According to other embodiments, the cosmetic composition is administered as a solution, a mist, or a spray. Each possibility represents a separated embodiment.

TABLE 1
Exemplary peptidomimetics by compound and position number

| Comp. | | | | | |
|---|---|---|---|---|---|
| No. | Position 1 | Position 2 | Position 3 | Position 4 | Position 5 |
| 1 | Gly | Phe(3-5-di-F) | D-Glu | 2-Nal | Phe(3-5-di-F) |
| 2 | Gly | Nle | D-Glu | Cha | Phe(3-5-di-F) |
| 3 | Gly | Nle | D-Glu | Tyr(3-NO₂) | Phe(3-5-di-F) |
| 4 | Gly | Nle | D-Glu | Phe(3-4-Cl) | Phe(3-5-di-F) |
| 5 | D-Ser | Nle | D-Glu | Cha | Phe(3-5-di-F) |
| 6 | Gly | beta-(2- | D-Glu | Phe(3-4-Cl) | 1-Nal |
| | | furyl)-Ala | | | |
| 7 | Gly | beta-(2- | D-Glu | Cha | Phe(3-5-di-F) |
| | | furyl)-Ala | | | |
| 8 | Gly | Phe(3-5-di-F) | D-Glu | Cha | Phe(3-5-di-F) |
| 9 | D-Ser | Phe(3-5-di-F) | D-Glu | 2-Nal | Phe(3-5-di-F) |
| 10 | Gly | hLeu | D-Glu | 2-Nal | Phe(3-5-di-F) |
| 11 | Gly | beta-(2- | D-Glu | 2-Nal | Phe(3-5-di-F) |
| | | furyl)-Ala | | | |
| 12 | Gly | Phe(3-5-di-F) | D-Glu | Cha | Phe(3-5-di-F) |
| 13 | Gly | beta-(2- | D-Glu | Phe(3-4-Cl) | Phe(3-5-di-F) |
| | | furyl)-Ala | | | |
| 14 | Gly | Tza | D-Glu | Phe(3-4-Cl) | 1-Nal |
| 15 | Gly | 2Thi | D-Glu | Phe(3-4-Cl) | 1-Nal |
| 16 | D-Nle | Met | D-Glu | Tyr(3-5-di-Iodo) | Hse |
| 17 | Gly | Phe(3-5-di-F) | D-Glu | Ala(9-Anth) | Hse |
| 18 | D-Ala | beta-(2- | D-Glu | Phe(3-4-Cl) | 1-Nal |
| | | furyl)-Ala | | | |
| 19 | Gly | Phe(3-5-di-F) | D-Glu | Phe(3-4-Cl) | Phe(3-5-di-F) |
| 20 | Gly | Phe(3-5-di-F) | D-Glu | Phe(3,4,5-F) | Phe(3-5-di-F) |
| 21 | Gly | Phe(3-5-di-F) | D-Glu | Tyr(3-NO₂) | Phe(3-5-di-F) |
| 22 | Gly | beta-(2- | D-Asp-[-1Nip] | Phe(3-4-Cl) | 1-Nal |
| | | furyl)-Ala | | | |
| 23 | D-Ala | beta-(2- | D-Glu | Phe(3-4-Cl) | L-Asp-[-GLY- |
| | | furyl)-Ala | | | GLY-GLY-3Abz] |
| 24 | Gly | Phe(3-5-di-F) | D-Glu | Tyr(3-5-di-Iodo) | Gly(indan-2-yl) |
| 25 | D-Ala | L-Nle(6-OH) | D-Glu | Phe(3-4-Cl) | 1-Nal |
| 26 | Gly | beta-(2- | D-Glu | Trp | Gly(indan-2-yl) |
| | | furyl)-Ala | | | |
| 27 | Gly | L-Nle(6-OH) | D-Glu | 1-Nal | Gly(indan-2-yl) |
| 28 | D-Ala | beta-(2- | D-Glu | Phe(3-4-Cl) | isoAad-[-1Nip- |
| | | furyl)-Ala | | | 3Abz] |
| 29 | D-Nle | L-Nle(6-OH) | D-Glu | Tyr(3-5-di-Iodo) | Hse |
| 30 | D-Ala | Phe(3-5-di-F) | D-Glu | Tyr(3-5-di-Iodo) | Gly(indan-2-yl) |
| 31 | D-aThr | Tza | D-Glu | Tyr(3-5-di-Iodo) | Gly(indan-2-yl) |
| 32 | D-aThr | Phe(3-5-di-F) | D-Glu | Tyr(3-5-di-Iodo) | Gly(indan-2-yl) |
| 33 | D-Nle | Tza | D-Glu | Tyr(3-5-di-Iodo) | Gly(indan-2-yl) |
| 34 | beta-(2- | Tza | D-Glu | Tyr(3-5-di-Iodo) | Gly(indan-2-yl) |
| | furyl)-D-Ala | | | | |
| 35 | D-Pal(4) | Tza | D-Glu | Tyr(3-5-di-Iodo) | Gly(indan-2-yl) |
| 36 | D-Ala | L-Nle(6-OH) | D-Glu | Tyr(3-5-di-Iodo) | Gly(indan-2-yl) |
| 37 | D-Nle(Me) | L-Nle(6-OH) | D-Glu | Tyr(3-5-di-Iodo) | Gly(indan-2-yl) |
| 38 | Gly | Cha | D-Glu | Tyr(3-5-di-Iodo) | Gly(indan-2-yl) |
| 39 | D-Abu | Pal(2) | D-Glu | Tyr(3-5-di-Iodo) | Gly(indan-2-yl) |
| 40 | D-Abu | Pal(3) | D-Glu | Tyr(3-5-di-Iodo) | Gly(indan-2-yl) |
| 41 | D-Ala | Pal(3) | D-Glu | Tyr(3-5-di-Iodo) | Gly(indan-2-yl) |
| 42 | D-hPhe | Ac3c | D-Glu | Tyr(3-5-di-Iodo) | Gly(indan-2-yl) |
| 43 | D-Pal(4) | Phe(3-F) | D-Glu | Tyr(3-5-di-Iodo) | Gly(indan-2-yl) |
| 44 | D-Pal(4) | Ala(cBu) | D-Glu | Tyr(3-5-di-Iodo) | Gly(indan-2-yl) |
| 45 | D-Pal(4) | Phe(3-Me) | D-Glu | Tyr(3-5-di-Iodo) | Gly(indan-2-yl) |
| 46 | D-Pal(4) | 2Thi | D-Glu | Tyr(3-5-di-Iodo) | Gly(indan-2-yl) |
| 47 | D-Pal(4) | Pal(3) | D-Glu | Tyr(3-5-di-Iodo) | Gly(indan-2-yl) |
| 48 | beta-(2- | beta-(2- | D-Glu | Tyr(3-5-di-Iodo) | Gly(indan-2-yl) |
| | furyl)-D-Ala | furyl)-Ala | | | |
| 49 | D-Pal(4) | aThr | D-Glu | Tyr(3-5-di-Iodo) | Gly(indan-2-yl) |
| 50 | D-hPhe | aThr | D-Glu | Tyr(3-5-di-Iodo) | Gly(indan-2-yl) |
| 51 | D-Pal(4) | Cha | D-Glu | Tyr(3-5-di-Iodo) | Gly(indan-2-yl) |
| 52 | D-Pal(4) | L-Nle(6-OH) | D-Glu | Tyr(3-5-di-Iodo) | Gly(indan-2-yl) |
| 53 | D-Pal(4) | Tza | D-Glu | Phe(2-NO₂) | Gly(indan-2-yl) |
| 54 | D-Pal(4) | Tza | D-Glu | Phe(4-guanidino) | Gly(indan-2-yl) |
| 55 | D-Pal(4) | Tza | D-Glu | Tyr(3-5-di-Iodo) | isoAad-[-Val-ol] |
| 56 | D-Pal(4) | Phe(3-OMe) | D-Glu | Tyr(3-5-di-Iodo) | Gly(indan-2-yl) |
| 57 | D-Pal(4) | Tza | D-Glu | Tyr(3-5-di-Bromo) | Gly(indan-2-yl) |
| 58 | D-aThr | Tza | D-Glu | L-isoAsp-[-3Abz] | Gly(indan-2-yl) |
| 59 | D-aThr | Tza | D-Glu | Tyr(3-NO₂) | Gly(indan-2-yl) |
| 60 | D-aThr | Tza | D-Glu | Phe(2-NO₂) | Gly(indan-2-yl) |
| 61 | D-Pal(4) | Pal(3) | D-Glu | Phe(2-NO₂) | Gly(indan-2-yl) |
| 62 | D-Pal(4) | Pal(3) | D-Glu | Tyr(3-NO₂) | Gly(indan-2-yl) |
| 63 | D-Pal(4) | Tza | D-Cys(O3H) | Tyr(3-5-di-Iodo) | Gly(indan-2-yl) |
| 64 | D-Pal(4) | Pal(3) | D-Glu | Tyr(3-5-di-Iodo) | Gly(indan-2-yl) |
| 65 | D-Pal(4) | Pal(3) | D-Cys(O3H) | Tyr(3-5-di-Iodo) | Gly(indan-2-yl) |
| 66 | D-Dap | Tza | D-Glu | Tyr(3-5-di-Iodo) | Gly(indan-2-yl) |
| 67 | D-Ala | Phe(3-OH) | D-Glu | Tyr(3-5-di-Iodo) | Gly(indan-2-yl) |
| 106 | D-aThr | 3Thi | D-Glu | Tyr(3-5-di-Iodo) | Gly(indan-2-yl) |
| 107 | D-aThr | Tza | D-Glu | 3-Bromo-5-fluoro- | Gly(indan-2-yl) |
| | | | | phenylalanine | |
| 108 | D-aThr | Tza | D-Glu | beta-(7-methoxy- | Gly(indan-2-yl) |
| | | | | coumarin-4-yl)-Ala | |
| 109 | D-aThr | Pal(3) | D-Glu | Tyr(3-5-di-Iodo) | Gly(indan-2-yl) |
| 110 | D-aThr | Gly(cPr) | D-Glu | Tyr(3-5-di-Iodo) | Gly(indan-2-yl) |
| 111 | D-Asn | Phe(3-OH) | D-Glu | Tyr(3-5-di-Iodo) | Gly(indan-2-yl) |
| 112 | D-aThr | Ala(cPr) | D-Glu | Tyr(3-5-di-Iodo) | Gly(indan-2-yl) |
| 113 | D-aThr | Ac3c | D-Glu | Tyr(3-5-di-Iodo) | Gly(indan-2-yl) |
| 114 | D-Ala | Phe(3-OEt) | D-Glu | Tyr(3-5-di-Iodo) | Gly(indan-2-yl) |
| 115 | D-aThr | Tza | D-Glu | Tyr(3-Me) | Gly(indan-2-yl) |
| 116 | D-aThr | Ala(cBu) | D-Glu | Tyr(3-5-di-Iodo) | Gly(indan-2-yl) |
| 117 | D-aThr | Phe(3-OEt) | D-Glu | Tyr(3-5-di-Iodo) | Gly(indan-2-yl) |
| 118 | D-aThr | aThr | D-Glu | Tyr(3-5-di-Iodo) | Gly(indan-2-yl) |
| 119 | D-aThr | Tza | D-Glu | Tyr(3-5-di-Iodo) | Asp(Ph(2-NH₂)) |
| 120 | D-aThr | Tza | D-Glu | Tyr(3-5-di-Iodo) | 1-Nal |
| 121 | D-aThr | Pal(3) | D-Abu | Tyr(3-5-di-Bromo) | Gly(indan-2-yl) |
| | | | (tetrazol-5-yl) | | |
| 122 | D-Pal(4) | Phe(3-OMe) | D-Glu | Tyr(3-5-di-Iodo) | 2-Nal |
| 123 | D-Pal(4) | Pal(3) | D-Abu | Tyr(3-5-di-Iodo) | Gly(indan-2-yl) |
| | | | (tetrazol-5-yl) | | |
| 124 | D-aThr | Pal(3) | D-Glu | Tyr(3-5-di-Iodo) | Aic |
| 125 | D-Ala | Pal(3) | D-Abu | Tyr(3-5-di-Iodo) | Gly(indan-2-yl) |
| | | | (tetrazol-5-yl) | | |
| 126 | Gly | Pal(3) | D-Abu | Tyr(3-5-di-Iodo) | Gly(indan-2-yl) |
| | | | (tetrazol-5-yl) | | |
| 127 | D-aThr | Pal(3) | D-Abu | Tyr(3-5-di-Iodo) | 1-Nal |
| | | | (tetrazol-5-yl) | | |
| 128 | D-Gln | Pal(3) | D-Abu | Tyr(3-5-di-Iodo) | Gly(indan-2-yl) |
| | | | (tetrazol-5-yl) | | |
| 129 | D-aThr | Ala | D-Abu | Tyr(3-5-di-Iodo) | Gly(indan-2-yl) |
| | | | (tetrazol-5-yl) | | |
| 130 | D-aThr | Pal(3) | D-Abu | Tyr(3-5-di-Iodo) | Ala |
| | | | (tetrazol-5-yl) | | |
| 131 | D-aThr | 2Thi | D-Glu | Tyr(3-5-di-Iodo) | Gly(indan-2-yl) |
| 132 | D-Nle(Me) | Tza | D-Glu | Tyr(3-5-di-Iodo) | Gly(indan-2-yl) |
| 133 | D-aThr | Tza | D-Glu | Phe(3-OCF₃) | Gly(indan-2-yl) |
| 134 | D-Pal(4) | Pal(3) | D-Abu | Tyr(3-5-di-Iodo) | 1-Nal |
| | | | (tetrazol-5-yl) | | |
| 135 | D-aThr | Pal(3) | D-Abu | Tyr(3-5-di-Iodo) | 2-Nal |
| | | | (tetrazol-5-yl) | | |
| 136 | D-aThr | Pal(3) | D-Abu | Tyr(3-5-di-Iodo) | 3-(4-Quinolyl)- |
| | | | (tetrazol-5-yl) | | alanine |
| 137 | D-Ala | Pal(3) | D-Abu | Tyr(3-5-di-Iodo) | 1-Nal |
| | | | (tetrazol-5-yl) | | |
| 138 | D-aThr | Pal(3) | D-Abu | Phe(3-5-di-Br) | 1-Nal |
| | | | (tetrazol-5-yl) | | |
| 139 | D-Ala | Pal(3) | D-Abu | Tyr(3-5-di-Iodo) | L-(7-azaTrp) |
| | | | (tetrazol-5-yl) | | |
| 140 | D-Ala | Pal(3) | D-Abu | Tyr(3-5-di-Iodo) | Phe(2-CF₃) |
| | | | (tetrazol-5-yl) | | |
| 141 | Nme-Gly | Pal(3) | D-Abu | Tyr(3-5-di-Iodo) | Gly(indan-2-yl) |
| | | | (tetrazol-5-yl) | | |
| 142 | D-aThr | beta-(2- | D-Abu | Tyr(3-5-di-Iodo) | Gly(indan-2-yl) |
| | | furyl)-D-Ala | (tetrazol-5-yl) | | |
| 143 | D-aThr | Tza | D-Glu | Bip | 1-Nal |
| 144 | D-aThr | Tza | D-Glu | 1-Nal | Gly(indan-2-yl) |
| 145 | D-aThr | Pal(3) | D-Abu | L-thyroxine | 1-Nal |
| | | | (tetrazol-5-yl) | | |
| 146 | D-Pal(4) | Pal(3) | D-Abu | Tyr(3-5-di-Iodo) | 3-(4-Quinolyl)- |
| | | | (tetrazol-5-yl) | | alanine |
| 147 | D-aThr | Tza | D-Glu | Nme-Tyr(3-Iodo) | Gly(indan-2-yl) |
| 148 | D-aThr | Tza | D-Glu | Bpa | 1-Nal |
| 149 | D-aThr | Tza | D-Glu | D-His(1-Bn) | 1-Nal |
| 150 | D-Gln | Pal(3) | D-Abu | Tyr(3-5-di-Iodo) | 3-(4-Quinolyl)- |
| | | | (tetrazol-5-yl) | | alanine |
| 151 | D-aThr | Pal(3) | D-Abu | Tyr(3-5-di-Iodo) | L-(7-azaTrp) |
| | | | (tetrazol-5-yl) | | |
| 152 | D-Pal(4) | Pal(3) | D-Abu | Tyr(3-5-di-Iodo) | L-(7-azaTrp) |
| | | | (tetrazol-5-yl) | | |
| 153 | D-Ala | Pal(3) | D-Abu | Tyr(3-5-di-Iodo) | 3-(4-Quinolyl)- |
| | | | (tetrazol-5-yl) | | alanine |
| 154 | D-Ala | Pal(3) | D-Abu | 5-Bromo-2-fluoro- | Phe(2-CF₃) |
| | | | (tetrazol-5-yl) | phenylalanine | |
| 155 | D-Ala | Pal(3) | D-Abu | Phe(2-OCF₃) | Phe(2-CF₃) |
| | | | (tetrazol-5-yl) | | |
| 156 | D-Ala | Pal(3) | D-Abu | Dip | Phe(2-CF₃) |
| | | | (tetrazol-5-yl) | | |
| 157 | D-Ala | Pal(3) | D-Abu | Dip | L-Gly(indan-2-yl) |
| | | | (tetrazol-5-yl) | | |
| 158 | D-aThr | Pal(3) | D-Glu | Dip | Phe(2-CF₃) |
| 159 | Propylamine | MPAA | D-Abu | Tyr(3-5-di-Iodo) | 1-Nal |
| | | | (tetrazol-5-yl) | | |
| 160 | D-Ala | Phe(3-5-di-F) | D-Glu | hTyr | Gly(indan-2-yl) |
| 161 | D-Ala | Phe(3-5-di-F) | D-Ser(Ac) | Tyr(3-5-di-Iodo) | Gly(indan-2-yl) |
| 162 | D-Ala | Phe(3-5-di-F) | D-Phe(3-NO₂) | Tyr(3-5-di-Iodo) | Gly(indan-2-yl) |
| 163 | D-Ala | Pal(3) | D-Abu | Phe(3-5-di-Br) | 1-Nal |
| | | | (tetrazol-5-yl) | | |
| 164 | D-Ala | Pal(3) | D-Glu | 2-Amino-3,3-bis(4- | Phe(2-CF₃) |
| | | | | fluorophenyl)propanoic | |
| | | | | acid | |
| 165 | D-aThr | Phe(3-5-di-F) | D-Glu | Dip | Gly(indan-2-yl) |
| 166 | D-Ala | Phe(3-OH) | D-Glu | Phe(2-OCF₃) | Gly(indan-2-yl) |
| 167 | D-Ala | Phe(3-5-di-F) | D-Glu | Nva(Ph) | Gly(indan-2-yl) |
| 168 | D-Ala | Pal(3) | D-Glu | 5-Bromo-2-fluoro- | Phe(2-CF₃) |
| | | | | phenylalanine | |
| 169 | D-Ala | Pal(3) | D-Cys | Tyr(3-5-di-Iodo) | Gly(indan-2-yl) |
| | | | (Methyl-2H- | | |
| | | | tetrazole)) | | |
| 170 | D-Ala | Phe(3-OH) | D-Glu | L-Phe(2-OCF₃) | 1-Nal |
| 171 | D-Ala | Pal(3) | D-Abu | Phe(2-OMe) | Phe(2-CF₃) |
| | | | (tetrazol-5-yl) | | |
| 172 | D-Ala | Phe(3-OH) | D-Glu | Phg(2-OMe) | 1-Nal |
| 173 | D-Ala | Pal(3) | D-Abu | 5-Bromo-2-fluoro- | Phe(2-OCF₃) |
| | | | (tetrazol-5-yl) | phenylalanine | |
| 174 | D-Gly(cPr) | Pal(3) | D-Abu | 5-Bromo-2-fluoro- | Phe(2-CF₃) |
| | | | (tetrazol-5-yl) | phenylalanine | |
| 175 | D-aThr | Pal(3) | D-Abu | 5-Bromo-2-fluoro- | Phe(2-CF₃) |
| | | | (tetrazol-5-yl) | phenylalanine | |
| 176 | D-Ala | Pal(3) | D-Abu | Phe(2-OCF₃) | Gly(indan-2-yl) |
| | | | (tetrazol-5-yl) | | |
| 177 | D-aThr | Pal(3) | D-Abu | Phe(2-OCF₃) | Phe(2-CF₃) |
| | | | (tetrazol-5-yl) | | |
| 178 | D-Gln | Pal(3) | D-Abu | Phe(2-OCF₃) | Phe(2-CF₃) |
| | | | (tetrazol-5-yl) | | |
| 179 | D-aThr | Pal(3) | D-Gla | 5-bromo-2-fluoro- | Phe(2-CF₃) |
| | | | | L-phenylalanine | |
| 180 | D-Gln | Pal(3) | D-Abu | Phe(2-OCF₃) | Phe(2-OCF₃) |
| | | | (tetrazol-5-yl) | | |
| 181 | D-aThr | Pal(3) | D-Abu | Phe(2-OMe) | Phg(2-OMe) |
| | | | (tetrazol-5-yl) | | |
| 182 | D-Gln | Pal(3) | D-Abu | Phe(2-OCF₃) | Ala(guanidino) |
| | | | (tetrazol-5-yl) | | |
| 183 | D-aThr | Pal(3) | D-Abu | Phe(2-OCF₃) | L-(7-azaTrp) |
| | | | (tetrazol-5-yl) | | |
| 184 | D-Gln | Pal(3) | D-Abu | Dip | Phe(2-CF₃) |
| | | | (tetrazol-5-yl) | | |
| 185 | D-aThr | Pal(3) | D-Abu | Phe(bR-OH) | Phe(2-CF₃) |
| | | | (tetrazol-5-yl) | | |
| 186 | D-aThr | Pal(3) | D-Abu | Phe(2-OMe) | Phe(2-CF₃) |
| | | | (tetrazol-5-yl) | | |
| 187 | D-aThr | Pal(3) | D-Abu | Phe(2-OCF₃) | Asp(Ph(2-NH₂)) |
| | | | (tetrazol-5-yl) | | |
| 188 | D-Ala | Pal(3) | D-Abu | hTyr | Phe(2-CF₃) |
| | | | (tetrazol-5-yl) | | |

### EXAMPLES

### Methods

### Microscale Thermophoresis

The dissociation constant (K_{d}) of the interaction between IL-17 and the peptides was studied by Microscale Thermophoresis (MST) using a Monolith NT.115 instrument. Biotinylated Human IL-17A/CTLA-8 Protein, His, Avi tag (ILA-H82Q1, Acro biosystem) was labeled using His-Tag Labeling Kit RED-tris-NTA 2^{nd} Generation (MO-L018, NanoTemper Technologies) according to the manufacturer's instructions. Binding experiments were conducted with 12.5 nM of protein in binding buffer (10 mM Hepes pH 7.4, 0.05% Tween20, and 2% DMSO) and peptide concentrations ranging from 0.031 to 100,000 nM. The interaction affinity and K_{d} were analyzed using the MO.Control Analysis software (version 2.2.4; NanoTemper Technologies).

### AlphaLISA Assay

Biotinylated Human IL-17A/CTLA-8 Protein, His, Avi tag (ILA-H82Q1) and IL-17RA/IL-17R Fc Chimera Protein Fc (#177-IR) were purchased from Acro biosystems and R&D Systems respectively; White 384 Opti plate (6007290), Protein A acceptor beads (AL101M), and streptavidin donor beads (67660002) were purchased from Perkin Elmer. Peptidomimetics were first dissolved in 100% DMSO, then diluted in assay buffer (50 mM Hepes, pH 7.4, 150 mM NaCl, 5 mM CaCl₂, 0.01% BSA, 0.05% Tween20) in 6 concentrations and then incubated with IL-17A at 0.746 nM for 1 h. 40 microliter of hIL-17R Fc at 8 nM was added to the plate for 1 hour incubation. After the incubation, the samples were transferred to a White 384 Opti plate in 20 microliter triplicates. Detection step was performed by first adding 10 microliter of Protein A acceptor beads for 1 hour and then 10 microliter of streptavidin donor beads at 20 microgram/ml final concentration. The plate was incubated for one hour at room temperature, then read on the Victor Nivo Multimode plate reader (Perkin Elmer). Positive controls (maximum signal) were wells with IL-17 without peptide, and negative control (minimum signal) were wells with assay buffer. A standard curve was prepared using 12 concentrations. The relative IC₅₀ values were determined using a four-parameter logistic. In the four-parameter logistic, bottom and top are defined as the plateaus of the curve, and H is the Hill Slope. Percent inhibition was determined by fitting the calculated AlphaLISA signal values to a standard 4-parameter logistic and non-linear regression analysis was performed using Prism software.

### Indirect Competitive ELISA

Coating buffer (BUF030A), blocking buffer (ELISA Sin block BUF034B), and TMB (BUF054B) were purchased from Bio Rad; and Pierce high sensitivity streptavidin-HRP (TS-21130) was obtained from Thermo Fisher.

High binding plates (CI-3590, Thermo) were coated with 8 nM rhIL-17 in coating buffer and incubated overnight at 25° C. Plates were washed 3× times by adding 300 μl of assay buffer (50 mM Hepes, pH 7.4, 150 mM NaCl, 5 mM CaCl₂, 0.05% Tween 20, 0.01% BSA, 2% DMSO) per well. Blocking buffer (150 μl/well) was added for incubation of 2 hours, while the peptides were preincubated for 1 hour with hIL-17A at 0.24 nM. Each peptide was tested at 8 different concentrations. As a positive control (maximum signal), wells were incubated with IL-17 without inhibitor, and as a negative control (minimum signal), wells were incubated with assay buffer and ELISA reagents. After the blocking step, wells were washed 3 times and 100 microliters of peptide: IL-17A complex were added to precoated plates in duplicate. After 1 hour of incubation, wells were washed and HRP (100 μl per well; 40 ng/ml) was added. After incubation and washing, TMB substrate was added (100 μl/well), and, following development of blue color, OD655 values were obtained using a Victor Nivo Multimode plate reader (PerkinElmer). In each experiment, a standard curve was prepared from 12 concentrations. Percent inhibition was determined by fitting the calculated signal values to a standard 4-parameter logistic and non-linear regression analysis was performed using Prism software.

### Primary Keratinocytes Cell Assay

Primary keratinocytes were cultured in a 384 well plate at 3,500 cells per well in a serum-free medium (C-20021, Promocell). 24 h after seeding, peptides were added to the cells at 7 different concentrations. Following the addition of the peptides, cells were treated with Human IL-17A (ILA-H82Q1, 3 ng/ml final concentration) and TNF-α (TNA-H4211, 10 ng/ml final concentration), or with TNF-α alone. Following 48 h of incubation, culture medium was collected from the cells and analyzed for IL-8 levels using AlphaLISA kit (AL224F, Perkin Elmer).

Percent of inhibition was determined by fitting the calculated signal values to a standard 4-parameter logistic and non-linear regression analysis using Prism software.

### Example 1: Inhibition of IL-17-IL-17R Interaction In-Vitro

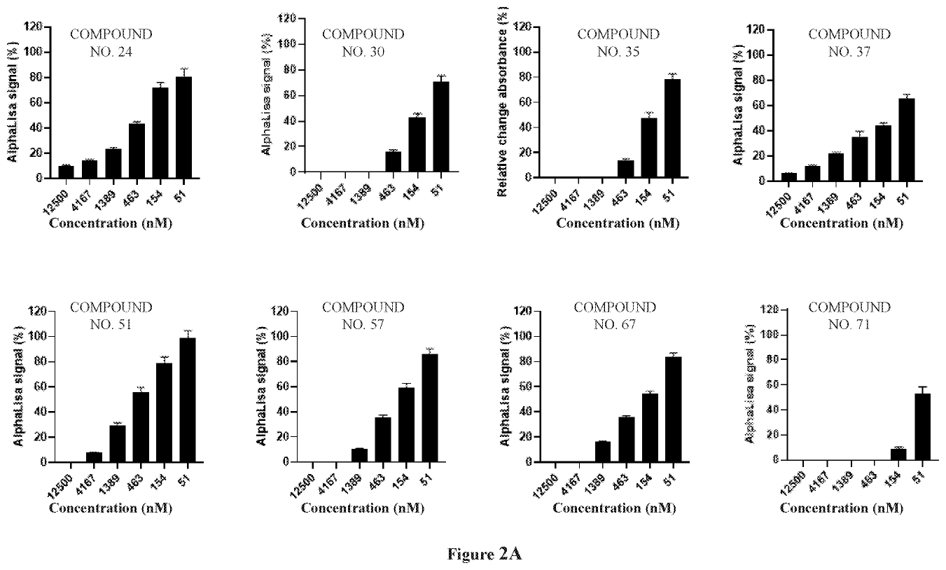

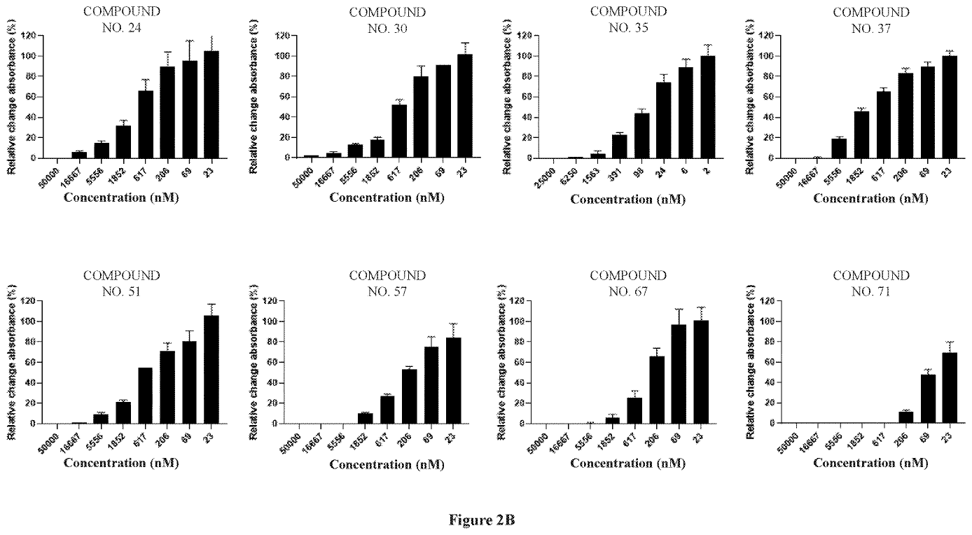

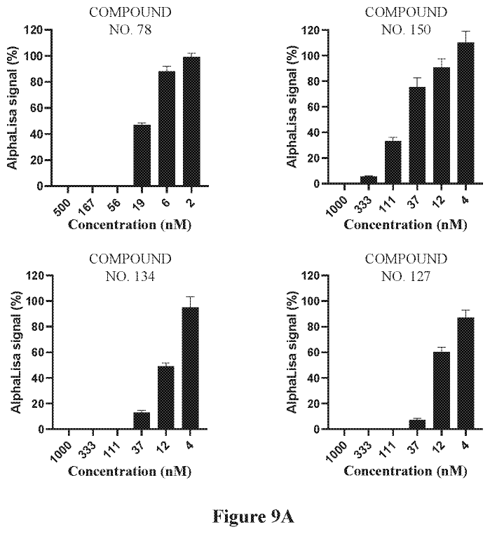

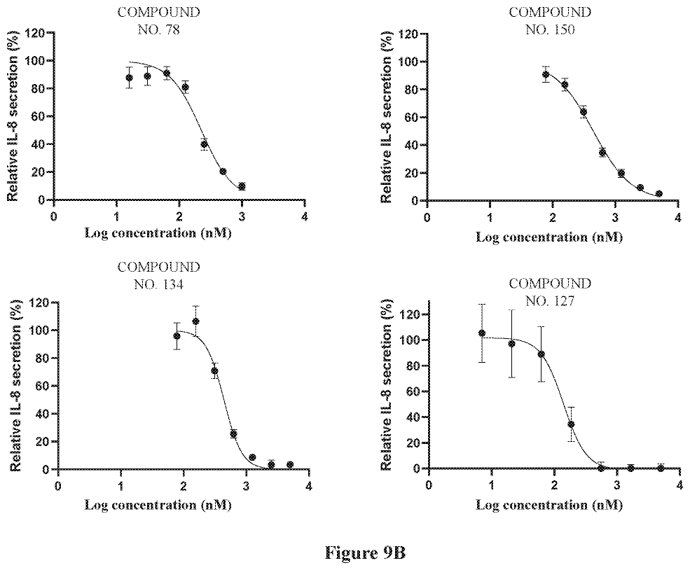

In an attempt to produce small molecules that operate as stable and effective inhibitors of IL-17-IL-17R interaction, an AI-based algorithm performed virtual screening on the unbound structure of Interleukin-17A homodimer (PDB: 4HR9), centered at a dynamic radius grid around Gln 94 of chain A of IL-17. Following the virtual screening, peptides comprising 5 amino acids linked by peptide bonds, were generated by the software, wherein the AA in each position was selected from a pool of about 600 different AAs that were selected by the software as potentially suitable for forming an IL-17 inhibitory peptidomimetic. The peptidomimetics which received the highest estimated IL-17 binding scores in the algorithm (out of about 1013 possibilities) were clustered to produce 251 independent cyclic peptidomimetics. The 251 peptidomimetics were synthesized by WuXi AppTec, using standard Fmoc chemistry, and were screened for actual inhibitory activity of the IL-17-IL-17R interaction. To this end, the peptidomimetics were subjected to an AlphaLISA Assay and/or to an indirect competitive ELISA assay at various peptide concentrations (6 and 8 different concentrations, respectively), according to the protocols set out in the Methods section hereinabove. Exemplary results of the AlphaLISA are presented in FIGS. 2A and 9A. Exemplary results of the indirect competitive ELISA are presented in FIG. 2B. The response curves of the different peptide concentrations were then used to determine 50% Inhibitory Concentration (IC₅₀) values for each peptidomimetic. For convenience of presentation, the IC₅₀ values are shown in Tables 2-4, as p(IC₅₀) values, attained by the formula: p(IC₅₀)=−1*log (IC₅₀), using a base 10 logarithm.

Unexpectedly, out of the 251 tested peptidomimetic compounds, 188 peptidomimetics were found to inhibit IL-17 at an efficacy value of about p(IC₅₀)>=4.5, the efficacy value being calculated as the average of AlphaLISA and ELISA results. The 75% ratio of active peptidomimetics is significantly larger than the expected one of 100,000-1,000,000 expected in random screening. In addition, within the 188 peptidomimetics, a select group of 34 peptidomimetics were identified with an efficacy value of p(IC₅₀) between 7 and 8, calculated in the same manner.

The p(IC₅₀) values according to the results of the AlphaLISA and/or the indirect competitive ELISA, of the 188 selected peptidomimetics, are set out in Table 2 below. All compounds have a head-to-tail cyclization.

TABLE 2
In-vitro IL-17A inhibitory activity of selected peptidomimetics

| | | p(IC₅₀) | |
|---|---|---|---|
| Comp. | | Alpha | |
| No. | Cyclic Peptidomimetic | Lisa | ELISA |
| 1 | Gly-(3-5-difluoro-phenylalanine)-(D-Glu)-((2-naphthyl)-L-Ala)-(3-5- | 6.1 | 5.6 |
| | difluoro-phenylalanine) | | |
| 2 | Gly-(L-Norleucine)-(D-Glu)-(Cyclohexylalanine)-(3-5-difluoro- | 5.0 | 4.6 |
| | phenylalanine) | | |
| 3 | Gly-(L-Norleucine)-(D-Glu)-(3-Nitro-Tyrosine)-(3-5-difluoro- | 6.4 | 4.6 |
| | phenylalanine) | | |
| 4 | Gly-(L-Norleucine)-(D-Glu)-(3-4 di-chloro-Phenylalanine)-(3-5-difluoro- | 6.0 | 5.0 |
| | phenylalanine) | | |
| 5 | (D-Serine)-(L-Norleucine)-(D-Glu)-(Cyclohexylalanine)-(3-5-difluoro- | 4.9 | 4.5 |
| | phenylalanine) | | |
| 6 | Gly-(L-2-Furylalanine)-(D-Glu)-(3-4-dichloro-phenylalanine)-((1-naphthyl)- | 6.4 | 4.9 |
| | L-Ala) | | |
| 7 | Gly-(L-2-Furylalanine)-(D-Glu)-(Cyclohexylalanine)-(3-5-difluoro- | 5.2 | 4.6 |
| | phenylalanine) | | |
| 8 | Gly-(3-5-difluoro-phenylalanine)-(D-Glu)-(Cyclohexylalanine)-(3-5- | 6.2 | 5.3 |
| | difluoro-phenylalanine) | | |
| 9 | (D-Serine)-(3-5-difluoro-phenylalanine)-(D-Glu)-((2-naphthyl)-L-Ala)-(3-5- | 4.8 | 5.4 |
| | difluoro-phenylalanine) | | |
| 10 | Gly-(5-methyl-L-norleucine)-(D-Glu)-((2-naphthyl)-L-Ala)-(3-5-difluoro- | 4.7 | 5.7 |
| | phenylalanine) | | |
| 11 | Gly-(L-2-Furylalanine)-(D-Glu)-((2-naphthyl)-L-Ala)-(3-5-difluoro- | — | 5.5 |
| | phenylalanine) | | |
| 12 | Gly-(3-5-difluoro-phenylalanine)-(D-Glu)-(Cyclohexylalanine)-(3-5- | 6.2 | 5.3 |
| | difluoro-phenylalanine) | | |
| 13 | Gly-(L-2-Furylalanine)-(D-Glu)-(3-4-dichloro-phenylalanine)-(3-5-difluoro- | 4.7 | 5.9 |
| | phenylalanine) | | |
| 14 | Gly-(Beta-(4-thiazolyl)-Ala)-(D-Glu)-(3-4-dichloro-phenylalanine)-((1- | 5.1 | 5.9 |
| | naphthyl)-L-Ala) | | |
| 15 | Gly-(Beta-(2-thienyl)-Ala)-(D-Glu)-(3-4-dichloro-phenylalanine)-((1- | 5.3 | 6.1 |
| | naphthyl)-L-Ala) | | |
| 16 | (D-Norleucine)-Met-(D-Glu)-(Tyr(3-5-di-Iodo))-(L-Homoserine) | 5.0 | 5.2 |
| 17 | Gly-(3-5-difluoro-phenylalanine)-(D-Glu)-(L-9-Anthrylalanine)-(L- | 5.4 | 5.0 |
| | Homoserine) | | |
| 18 | (D-Ala)-(L-2-Furylalanine)-(D-Glu)-(3-4-dichloro-phenylalanine)-((1- | 5.1 | 6.2 |
| | naphthyl)-L-Ala) | | |
| 19 | Gly-(3-5-difluoro-phenylalanine)-(D-Glu)-(3-4-dichloro-phenylalanine)-(3- | 5.4 | 5.3 |
| | 5-difluoro-phenylalanine) | | |
| 20 | Gly-(3-5-difluoro-phenylalanine)-(D-Glu)-(3,4,5-fluoro-phenylalanine)-(3-5- | 5.0 | 5.3 |
| | difluoro-phenylalanine) | | |
| 21 | Gly-(3-5-difluoro-phenylalanine)-(D-Glu)-(3-Nitro-Tyrosine)-(3-5-difluoro- | 5.7 | 5.8 |
| | phenylalanine) | | |
| 22 | Gly-(L-2-Furylalanine)-(D-Asp-[-1Nip])-(3-4-dichloro-phenylalanine)-((1- | 5.1 | 4.8 |
| | naphthyl)-L-Ala) | | |
| 23 | D-Ala-(beta-(2-furyl)-Ala)-(D-Glu)-Phe(3-4-Cl)-(IsoAsp-[Gly-Gly-Gly- | 5.1 | — |
| | 3Abz]) | | |
| 24 | Gly-(3-5-difluoro-phenylalanine)-(D-Glu)-(Tyr(3-5-di-Iodo))-(L-2- | 5.7 | 6.1 |
| | indanylglycine) | | |
| 25 | (D-Ala)-(6-hydroxynorleucine)-(D-Glu)-(3-4-dichloro-phenylalanine)-((1- | 4.8 | 6.0 |
| | naphthyl)-L-Ala) | | |
| 26 | Gly-(L-2-Furylalanine)-(D-Glu)-Trp-(L-2-indanylglycine) | 5.0 | 5.5 |
| 27 | Gly-(6-hydroxynorleucine)-(D-Glu)-((1-naphthyl)-L-Ala)-(L-2- | 6.0 | 5.3 |
| | indanylglycine) | | |
| 28 | (D-Ala)-(L-2-Furylalanine)-(D-Glu)-(3-4-dichloro-phenylalanine)-(Aad-[- | 4.6 | 4.2 |
| | 1Nip-3Abz]) | | |
| 29 | (D-Norleucine)-(6-hydroxynorleucine)-(D-Glu)-(Tyr(3-5-di-Iodo))-(L- | 4.9 | 4.8 |
| | Homoserine) | | |
| 30 | (D-Ala)-(3-5-difluoro-phenylalanine)-(D-Glu)-(Tyr(3-5-di-Iodo))-(L-2- | 7.1 | 6.2 |
| | indanylglycine) | | |
| 31 | (D-Allothreonine)-(Beta-(4-thiazolyl)-Ala)-(D-Glu)-(Tyr(3-5-di-Iodo))-(L-2- | 7.0 | 6.7 |
| | indanylglycine) | | |
| 32 | (D-Allothreonine)-(3-5-difluoro-phenylalanine)-(D-Glu)-(Tyr(3-5-di-Iodo))- | 6.8 | 6.4 |
| | (L-2-indanylglycine) | | |
| 33 | (D-Norleucine)-(Beta-(4-thiazolyl)-Ala)-(D-Glu)-(Tyr(3-5-di-Iodo))-(L-2- | 7.0 | 6.7 |
| | indanylglycine) | | |
| 34 | (D-2-Furylalanine)-(Beta-(4-thiazolyl)-Ala)-(D-Glu)-(Tyr(3-5-di-Iodo))-(L- | 6.6 | 6.2 |
| | 2-indanylglycine) | | |
| 35 | (D-4-pyridyl-Ala)-(Beta-(4-thiazolyl)-Ala)-(D-Glu)-(Tyr(3-5-di-Iodo))-(L-2- | 6.8 | 6.3 |
| | indanylglycine) | | |
| 36 | (D-Ala)-(6-hydroxynorleucine)-(D-Glu)-(Tyr(3-5-di-Iodo))-(L-2- | 5.9 | 5.4 |
| | indanylglycine) | | |
| 37 | (R-2-aminoheptanoic-acid)-(6-hydroxynorleucine)-(D-Glu)-(Tyr(3-5-di- | 6.5 | 5.7 |
| | Iodo))-(L-2-indanylglycine) | | |
| 38 | Gly-(Cyclohexylalanine)-(D-Glu)-(Tyr(3-5-di-Iodo))-(L-2-indanylglycine) | 6.9 | 5.9 |
| 39 | (D-2-alpha-Aminobutyric-acid)-(L-2-pyridyl-Ala)-(D-Glu)-(Tyr(3-5-di- | 6.2 | 5.9 |
| | Iodo))-(L-2-indanylglycine) | | |
| 40 | (D-2-alpha-Aminobutyric-acid)-(L-3-pyridyl-Ala)-(D-Glu)-(Tyr(3-5-di- | 6.5 | 6.3 |
| | Iodo))-(L-2-indanylglycine) | | |
| 41 | (D-Ala)-(L-3-pyridyl-Ala)-(D-Glu)-(Tyr(3-5-di-Iodo))-(L-2-indanylglycine) | 6.5 | 5.9 |
| 42 | (D-homophenylalanine)-(1-Aminocyclopropane-1-carboxylic acid)-(D-Glu)- | 6.1 | 5.7 |
| | (Tyr(3-5-di-Iodo))-(L-2-indanylglycine) | | |
| 43 | (D-4-pyridyl-Ala)-(3-fluoro-L-phenylalanine)-(D-Glu)-(Tyr(3-5-di-Iodo))- | 6.5 | 6.2 |
| | (L-2-indanylglycine) | | |
| 44 | (D-4-pyridyl-Ala)-(Beta-Cyclobutyl-L-Ala)-(D-Glu)-(Tyr(3-5-di-Iodo))-(L- | 6.8 | 6.4 |
| | 2-indanylglycine) | | |
| 45 | (D-4-pyridyl-Ala)-(3-methyl-L-phenylalanine)-(D-Glu)-(Tyr(3-5-di-Iodo))- | 6.5 | 5.3 |
| | (L-2-indanylglycine) | | |
| 46 | (D-4-pyridyl-Ala)-(Beta-(2-thienyl)-Ala)-(D-Glu)-(Tyr(3-5-di-Iodo))-(L-2- | 6.7 | 6.7 |
| | indanylglycine) | | |
| 47 | (D-4-pyridyl-Ala)-(L-3-pyridyl-Ala)-(D-Glu)-(Tyr(3-5-di-Iodo))-(L-2- | 6.5 | 5.5 |
| | indanylglycine) | | |
| 48 | (D-2-Furylalanine)-(L-2-Furylalanine)-(D-Glu)-(Tyr(3-5-di-Iodo))-(L-2- | 6.8 | 6.2 |
| | indanylglycine) | | |
| 49 | (D-4-pyridyl-Ala)-(L-Allothreonine)-(D-Glu)-(Tyr(3-5-di-Iodo))-(L-2- | 6.5 | 5.9 |
| | indanylglycine) | | |
| 50 | (D-homophenylalanine)-(L-Allothreonine)-(D-Glu)-(Tyr(3-5-di-Iodo))-(L-2- | 6.2 | 5.5 |
| | indanylglycine) | | |
| 51 | (D-4-pyridyl-Ala)-(Cyclohexylalanine)-(D-Glu)-(Tyr(3-5-di-Iodo))-(L-2- | 6.9 | 6.0 |
| | indanylglycine) | | |
| 52 | (D-4-pyridyl-Ala)-(6-hydroxynorleucine)-(D-Glu)-(Tyr(3-5-di-Iodo))-(L-2- | 5.9 | 5.7 |
| | indanylglycine) | | |
| 53 | (D-4-pyridyl-Ala)-(Beta-(4-thiazolyl)-Ala)-(D-Glu)-(2-nitro-phenylalanine)- | 5.9 | 6.0 |
| | (L-2-indanylglycine) | | |
| 54 | (D-4-pyridyl-Ala)-(Beta-(4-thiazolyl)-Ala)-(D-Glu)-(4- | 5.2 | 6.0 |
| | Guanidinophenylalanine)-(L-2-indanylglycine) | | |
| 55 | (D-4-pyridyl-Ala)-(Beta-(4-thiazolyl)-Ala)-(D-Glu)-(Tyr(3-5-di-Iodo))- | 5.7 | — |
| | (isoAad-[-Val-ol]) | | |
| 56 | (D-4-pyridyl-Ala)-(3-methoxy-L-phenylalanine)-(D-Glu)-(Tyr(3-5-di-Iodo))- | 7.1 | 6.7 |
| | (L-2-indanylglycine) | | |
| 57 | (D-4-pyridyl-Ala)-(Beta-(4-thiazolyl)-Ala)-(D-Glu)-(3-5-diBromo-tyrosine)- | 6.3 | 6.7 |
| | (L-2-indanylglycine) | | |
| 58 | (D-Allothreonine)-(Beta-(4-thiazolyl)-Ala)-(D-Glu)-(L-isoAsp-[-3Abz])-(L- | 5.1 | 5.0 |
| | 2-indanylglycine) | | |
| 59 | (D-Allothreonine)-(Beta-(4-thiazolyl)-Ala)-(D-Glu)-(3-Nitro-L-tyrosine)-(L- | 5.8 | 6.3 |
| | 2-indanylglycine) | | |
| 60 | (D-Allothreonine)-(Beta-(4-thiazolyl)-Ala)-(D-Glu)-(2-nitro-phenylalanine)- | 6.1 | 5.5 |
| | (L-2-indanylglycine) | | |
| 61 | (D-4-pyridyl-Ala)-(L-3-pyridyl-Ala)-(D-Glu)-(2-nitro-phenylalanine)-(L-2- | 6.0 | 5.1 |
| | indanylglycine) | | |
| 62 | (D-4-pyridyl-Ala)-(L-3-pyridyl-Ala)-(D-Glu)-(3-Nitro-L-tyrosine)-(L-2- | 5.7 | 5.1 |
| | indanylglycine) | | |
| 63 | (D-4-pyridyl-Ala)-(Beta-(4-thiazolyl)-Ala)-(D-Cysteic Acid)-(Tyr(3-5-di- | 6.0 | 5.5 |
| | Iodo))-(L-2-indanylglycine) | | |
| 64 | (D-4-pyridyl-Ala)-(L-3-pyridyl-Ala)-(D-Glu)-(Tyr(3-5-di-Iodo))-(L-2- | 6.1 | 6.3 |
| | indanylglycine) | | |
| 65 | (D-4-pyridyl-Ala)-(L-3-pyridyl-Ala)-(D-Cysteic Acid)-(Tyr(3-5-di-Iodo))- | 5.7 | 5.5 |
| | (L-2-indanylglycine) | | |
| 66 | (D-Dap)-(Beta-(4-thiazolyl)-Ala)-(D-Glu)-(Tyr(3-5-di-Iodo))-(L-2- | 5.6 | 5.7 |
| | indanylglycine) | | |
| 67 | (D-Ala)-(3-hydroxy-phenylalanine)-(D-Glu)-(Tyr(3-5-di-Iodo))-(L-2- | 6.7 | 6.4 |
| | indanylglycine) | | |
| 68 | (D-Ala)-(3-5-difluoro-phenylalanine)-(D-Glu)-(3-3-diphenyl-L-Ala)-(L-2- | 6.0 | 6.3 |
| | indanylglycine) | | |
| 69 | (D-4-pyridyl-Ala)-(3-hydroxy-phenylalanine)-(D-Glu)-(Tyr(3-5-di-Iodo))- | 6.7 | 6.7 |
| | (L-2-indanylglycine) | | |
| 70 | (D-Ala)-(3-hydroxy-phenylalanine)-(D-Glu)-(5-methoxy-L-tryptophan)-(L- | 5.2 | 5.0 |
| | 2-indanylglycine) | | |
| 71 | (D-Allothreonine)-(Beta-(4-thiazolyl)-Ala)-(D-Abu(tetrazol-5-yl))-(Tyr(3-5- | 7.5 | 7.3 |
| | di-Iodo))-(L-2-indanylglycine) | | |
| 72 | (Gly-Cha-(D-Glu)-(4-Carboxyphenylalanine)-Gly(indan-2-yl) | 5.4 | 5.7 |
| 73 | [*] (Propylamine)-(MPAA)-(D-Glu)-(Tyr(3-5-di-Iodo))-(L-2-indanylglycine) | 6.0 | 5.7 |
| 74 | [*] (Cysteamine)-((1,3-Dimethylbenzene)-3-sulfanylpropanoic acid)-(D-Glu)- | 6.1 | 4.6 |
| | (Tyr(3-5-di-Iodo))-(L-2-indanylglycine) | | |
| 75 | (D-Ala)-(3-hydroxy-phenylalanine)-(D-Glu)-(2-(trifluoromethoxy)-L- | 6.3 | 6.3 |
| | phenylalanine)-(L-2-indanylglycine) | | |
| 76 | (D-Ala)-(3-hydroxy-phenylalanine)-(D-Glu)-(L-2-Cyanophenylalanine)-(L- | 5.6 | 5.8 |
| | 2-indanylglycine) | | |
| 77 | (D-Ala)-(3-hydroxy-phenylalanine)-(D-Glu)-(Tyr(3-5-di-Iodo))-(N-(2-4- | 5.0 | — |
| | dimethoxybenzyl)-Gly) | | |
| 78 | (D-Allothreonine)-(L-3-pyridyl-Ala)-(D-Abu(tetrazol-5-yl))-(Tyr(3-5-di- | 7.6 | 7.8 |
| | Iodo))-(L-2-indanylglycine) | | |
| 79 | (D-Allothreonine)-(Beta-(4-thiazolyl)-Ala)-(D-Abu(tetrazol-5-yl))-(Tyr(3-5- | 5.4 | 5.3 |
| | di-Chloro))-(L-2-indanylglycine) | | |
| 80 | (D-4-pyridyl-Ala)-(3-methoxy-L-phenylalanine)-(D-Glu)-(Tyr(3-5-di-Iodo)- | 5.7 | — |
| | (L-isoGlu-[Tyr(3-I)-DhPhe-Ser(Me)-Dcit]) | | |
| 81 | (D-Ala)-(3-ethoxy-L-phenylalanine)-(D-Abu(tetrazol-5-yl)-(Tyr(3-5-di- | 6.6 | 6.0 |
| | Iodo))-(L-2-indanylglycine) | | |
| 82 | (D-Allothreonine)-(Beta-(4-thiazolyl)-Ala)-(D-Glu)-(3-4-methoxy-L- | 5.1 | — |
| | phenylalanine)-(L-2-indanylglycine) | | |
| 83 | (D-Allothreonine)-(Beta-(4-thiazolyl)-Ala)-(D-Glu)-(3-4-5-trifluoro- | 5.0 | — |
| | phenylalanine)-(L-2-indanylglycine) | | |
| 84 | (D-Allothreonine)-(3-5-difluoro-phenylalanine)-(D-Abu(tetrazol-5-yl))- | 6.8 | 6.5 |
| | (Tyr(3-5-di-Iodo))-(L-2-indanylglycine) | | |
| 85 | (D-Allothreonine)-(Beta-(4-thiazolyl)-Ala)-(D-Glu)-(4-amino-L- | 5.6 | 4.9 |
| | phenylalanine)-(L-2-indanylglycine) | | |
| 86 | (D-Allothreonine)-(Beta-(4-thiazolyl)-Ala)-(D-Glu)-(3-4-dichloro- | 5.3 | 4.9 |
| | phenylalanine)-(L-2-indanylglycine) | | |
| 87 | (D-Allothreonine)-(Beta-(4-thiazolyl)-Ala)-(D-Glu)-(L-(7-azaTrp))-(L-2- | 5.8 | 5.3 |
| | indanylglycine) | | |
| 88 | (D-Allothreonine)-(3-hydroxy-L-phenylalanine)-(D-Abu(tetrazol-5-yl))- | 6.8 | 6.5 |
| | (Tyr(3-5-di-Iodo))-(L-2-indanylglycine) | | |
| 89 | (beta-(2-furyl)-D-Ala)-(Beta-(4-thiazolyl)-Ala)-(D-Abu(tetrazol-5-yl))- | 5.6 | 5.1 |
| | (Tyr(3-5-di-Iodo))-(L-2-indanylglycine) | | |
| 90 | (D-Allothreonine)-(Beta-(4-thiazolyl)-Ala)-(D-Glu)-(S-xanthyl-L-cysteine)- | 5.2 | — |
| | (L-2-indanylglycine) | | |
| 91 | (D-4-pyridyl-alanine)(3-ethoxy-L-phenylalanine)e(Tyr(3-5-di-Iodo))(L-2- | 6.9 | 6.6 |
| | indanylglycine) | | |
| 92 | (D-2-alpha-Aminobutyric-acid)-(L-3-pyridyl-alanine)-(D-Abu(tetrazol-5- | 7.4 | 7.1 |
| | yl))-(Tyr(3-5-di-Iodo))-(L-2-indanylglycine) | | |
| 93 | (R-2-aminoheptanoic-acid)-(L-3-pyridyl-alanine)-(D-Abu(tetrazol-5-yl))- | 7.2 | 6.8 |
| | (Tyr(3-5-di-Iodo))-(L-2-indanylglycine) | | |
| 94 | (D-Ala)-(3-hydroxy-phenylalanine)-(D-Glu)-(Tyr(3-5-di-Iodo))-(Nal]) | 7.1 | 6.7 |
| 95 | (D-Ala)-(3-hydroxy-phenylalanine)-(D-Glu)-(Tyr(3-5-di-Iodo))(Trp) | 6.4 | 5.8 |
| 96 | (D-Allothreonine)-(Beta-(4-thiazolyl)-Ala)-(D-Glu)-(Tyr(3-5-di-Methyl))- | 6.0 | 5.5 |
| | (L-2-indanylglycine) | | |
| 97 | (D-Glutamine)-(3-hydroxy-phenylalanine)-(D-Glu)-(Tyr(3-5-di-Iodo))-(L-2- | 7.3 | 6.7 |
| | indanylglycine) | | |
| 98 | (D-Allothreonine)-(Beta-(4-thiazolyl)-Ala)-(D-2-aminoadipic acid)-(Tyr(3- | 6.8 | 6.7 |
| | 5-di-Iodo))-(L-2-indanylglycine) | | |
| 99 | (D-Allothreonine)-(Beta-(4-thiazolyl)-Ala)-(D-Glu)-(pentafluoro-L- | 5.9 | 6.0 |
| | phenylalanine)-(L-2-indanylglycine) | | |
| 100 | (D-2-alpha-Aminobutyric-acid)-(3-methoxy-L-phenylalanine)-(D-Glu)-(3-5- | 7.0 | 6.9 |
| | di-iodo-tyrosine)-(L-2-indanylglycine) | | |
| 101 | (D-Glutamine)-(3-methoxy-L-phenylalanine)-(D-Glu)-(Tyr(3-5-di-Iodo))-(L- | 7.0 | 7.2 |
| | 2-indanylglycine) | | |
| 102 | (D-Allothreonine)-(Beta-(4-thiazolyl)-Ala)-(D-Glu)-(Tyr(3-Iodo))-(L-2- | 6.8 | 5.8 |
| | indanylglycine) | | |
| 103 | (D-Allothreonine)-(Beta-(4-thiazolyl)-Ala)-(D-Glu)-(1-thyroxine)-(L-2- | 6.6 | 5.9 |
| | indanylglycine) | | |
| 104 | (D-Allothreonine)-(Beta-(4-thiazolyl)-Ala)-(D-Glu)-((S)-2-Methoxy- | 6.2 | 5.4 |
| | phenylglycine)-(L-2-indanylglycine) | | |
| 105 | (D-Allothreonine)-(L-3-thienylalanine)-(D-Glu)-(Tyr(3-5-di-Iodo))-(L-2- | 6.6 | 6.4 |
| | indanylglycine) | | |
| 106 | (D-Allothreonine)-(Beta-(4-thiazolyl)-Ala)-(D-Glu)-(Tyr(3-5-di-Iodo))-(2- | 6.5 | 6.3 |
| | (trifluoromethyl)-L-phenylalanine) | | |
| 107 | (D-Allothreonine)-(Beta-(4-thiazolyl)-Ala)-(D-Glu)-(3-Bromo-5-fluoro- | 6.5 | — |
| | phenylalanine)-(L-2-indanylglycine) | | |
| 108 | (D-Allothreonine)-(Beta-(4-thiazolyl)-Ala)-(D-Glu)-(beta-(7-methoxy- | 5.9 | 5.8 |
| | coumarin-4-yl)-Ala)-(L-2-indanylglycine) | | |
| 109 | (D-Allothreonine)-(L-3-pyridyl-alanine)-(D-Glu)-(Tyr(3-5-di-Iodo))-(L-2- | 6.6 | 6.8 |
| | indanylglycine) | | |
| 110 | (D-Allothreonine)-(L-Cyclopropylglycine)-(D-Glu)-(Tyr(3-5-di-Iodo))-(L-2- | 6.3 | 5.8 |
| | indanylglycine) | | |
| 111 | (D-Asparagnine)-(3-hydroxy-phenylalanine)-(D-Glu)-(Tyr(3-5-di-Iodo))-(L- | 6.1 | 5.4 |
| | 2-indanylglycine) | | |
| 112 | (D-Allothreonine)-(L-Cyclopropylalanine)-(D-Glu)-(Tyr(3-5-di-Iodo))-(L-2- | 6.7 | 6.4 |
| | indanylglycine) | | |
| 113 | (D-Allothreonine)-(1-Aminocyclopropane-1-carboxylic acid)-(D-Glu)- | 5.0 | 4.5 |
| | (Tyr(3-5-di-Iodo))-(L-2-indanylglycine) | | |
| 114 | (D-Ala)-(3-ethoxy-L-phenylalanine)-(D-Glu)-(Tyr(3-5-di-Iodo))-(L-2- | 7.1 | 7.0 |
| | indanylglycine) | | |
| 115 | (D-Allothreonine)-(Beta-(4-thiazolyl)-Ala)-(D-Glu)-(3-methyl-L-tyrosine)- | 5.7 | 5.5 |
| | (L-2-indanylglycine) | | |
| 116 | (D-Allothreonine)-(Beta-Cyclobutyl-L-Alanine)-(D-Glu)-(Tyr(3-5-di-Iodo))- | 6.4 | 6.0 |
| | (L-2-indanylglycine) | | |
| 117 | (D-Allothreonine)-(3-ethoxy-L-phenylalanine)-(D-Glu)-(Tyr(3-5-di-Iodo))- | 7.3 | 6.9 |
| | (L-2-indanylglycine) | | |
| 118 | (D-Allothreonine)-(Allothreonine)-(D-Glu)-(Tyr(3-5-di-Iodo))-(L-2- | 6.7 | 6.2 |
| | indanylglycine) | | |
| 119 | (D-Allothreonine)-(Beta-(4-thiazolyl)-Ala)-(D-Glu)-(Tyr(3-5-di-Iodo))-(L- | 6.2 | 5.6 |
| | Kynurenine) | | |
| 120 | (D-Allothreonine)-(Beta-(4-thiazolyl)-Ala)-(D-Glu)-(Tyr(3-5-di-Iodo))-((1- | 6.5 | 6.3 |
| | naphthyl)-L-alanine) | | |
| 121 | (D-Allothreonine)-(L-3-pyridyl-alanine)-(D-Abu(tetrazol-5-yl))-(Tyr(3-5-di- | 7.5 | 7.3 |
| | Bromo)-(L-2-indanylglycine) | | |
| 122 | (D-4-pyridyl-alanine)-(3-methoxy-L-phenylalanine)-(D-Glu)-(Tyr(3-5-di- | 6.6 | 6.8 |
| | Iodo))-(2-naphthyl)-L-alanine)) | | |
| 123 | (D-4-pyridyl-alanine)-(L-3-pyridyl-alanine)-(D-Abu(tetrazol-5-yl))-(Tyr(3-5- | 7.6 | 7.7 |
| | di-Iodo))-(L-2-indanylglycine) | | |
| 124 | (D-Allothreonine)-(L-3-pyridyl-alanine)-(D-Glu)-(Tyr(3-5-di-Iodo)-(Aic) | 5.0 | — |
| 125 | (D-Alanine)-(L-3-pyridyl-alanine)-(D-Abu(tetrazol-5-yl))-(Tyr(3-5-di-Iodo))- | 7.7 | 7.2 |
| | (L-2-indanylglycine) | | |
| 126 | Gly-(L-3-pyridyl-alanine)-(D-Abu(tetrazol-5-yl))-(Tyr(3-5-di-Iodo))-(L-2- | 7.1 | 6.6 |
| | indanylglycine) | | |
| 127 | (D-Allothreonine)-(L-3-pyridyl-alanine)-(D-Abu(tetrazol-5-yl))-(Tyr(3-5-di- | 7.9 | 7.5 |
| | Iodo))-(1-naphthyl)-L-alanine) | | |
| 128 | (D-Glutamine)-(L-3-pyridyl-alanine)-(D-Abu(tetrazol-5-yl))-(Tyr(3-5-di- | 6.8 | 6.5 |
| | Iodo))-(L-2-indanylglycine) | | |
| 129 | (D-Allothreonine)-(Alanine)-(D-Abu(tetrazol-5-yl))-(Tyr(3-5-di-Iodo))-(L-2- | 6.7 | 4.8 |
| | indanylglycine) | | |
| 130 | (D-Allothreonine)-(L-3-pyridyl-alanine)-(D-Abu(tetrazol-5-yl))-(Tyr(3-5-di- | 4.9 | 5.0 |
| | Iodo))-(Alanine) | | |
| 131 | (D-Allothreonine)-(Beta-(2-thienyl)-Ala)-(D-Glu)-(Tyr(3-5-di-Iodo))-(L-2- | 6.8 | 6.3 |
| | indanylglycine) | | |
| 132 | (R-2-aminoheptanoic-acid)-(Beta-(4-thiazolyl)-Ala)-(D-Glu)-(Tyr(3-5-di- | 6.8 | 6.8 |
| | Iodo))-(L-2-indanylglycine) | | |
| 133 | (D-Allothreonine)-(Beta-(4-thiazolyl)-Ala)-(D-Glu)-(3-(trifluoromethoxy)-L- | 6.1 | 6.0 |
| | phenylalanine)-(L-2-indanylglycine) | | |
| 134 | (D-4-pyridyl-alanine)-(L-3-pyridyl-alanine)-(D-Abu(tetrazol-5-yl))-(Tyr(3-5- | 7.7 | 7.9 |
| | di-Iodo))-(1-naphthyl)-L-alanine) | | |
| 135 | (D-Allothreonine)-(L-3-pyridyl-alanine)-(D-Abu(tetrazol-5-yl))-(Tyr(3-5-di- | 7.4 | 7.2 |
| | Iodo))-(2-naphthyl)-L-alanine) | | |
| 136 | (D-Allothreonine)-(L-3-pyridyl-alanine)-(D-Abu(tetrazol-5-yl))-(Tyr(3-5-di- | 7.0 | 7.9 |
| | Iodo))-(3-(4-Quinolyl))-L-alanine) | | |
| 137 | (D-Ala)-(L-3-pyridyl-alanine)-(D-Abu(tetrazol-5-yl))-(Tyr(3-5-di-Iodo))-(1- | 7.2 | 7.0 |
| | naphthyl)-L-alanine) | | |
| 138 | (D-Allothreonine)-(L-3-pyridyl-alanine)-(D-Abu(tetrazol-5-yl))-(Phe(3-5-di- | 6.8 | 6.7 |
| | Br))-(1-naphthyl)-L-alanine) | | |
| 139 | (D-Ala)-(L-3-pyridyl-alanine)-(D-Abu(tetrazol-5-yl))-(Tyr(3-5-di-Iodo))-(L- | 7.1 | 5.3 |
| | (7-azaTrp)) | | |
| 140 | (D-Ala)-(L-3-pyridyl-alanine)-(D-Abu(tetrazol-5-yl))-(Tyr(3-5-di-Iodo))-(2- | 7.6 | 7.3 |
| | (trifluoromethyl)-L-phenylalanine) | | |
| 141 | (Nmethyl-Gly)-(L-3-pyridyl-alanine)-(D-Abu(tetrazol-5-yl))-(Tyr(3-5-di- | 5.1 | — |
| | Iodo))-(L-2-indanylglycine) | | |
| 142 | (D-Allothreonine)-(beta-(2-furyl)-D-Ala)-(D-Abu(tetrazol-5-yl))-(Tyr(3-5- | 6.7 | 5.5 |
| | di-Iodo))-(L-2-indanylglycine) | | |
| 143 | (D-Allothreonine)-(Beta-(4-thiazolyl)-Ala)-(D-Glu)-(4-phenyl-Phe)-((1- | 5.8 | 6.0 |
| | naphthyl)-L-alanine) | | |
| 144 | (D-Allothreonine)-(Beta-(4-thiazolyl)-Ala)-(D-Glu)-((1-naphthyl)-L- | 5.1 | 4.9 |
| | alanine))-(L-2-indanylglycine) | | |
| 145 | (D-Allothreonine)-(L-3-pyridyl-alanine)-(D-Abu(tetrazol-5-yl))-(L-thyroxine))- | 6.7 | 6.9 |
| | (1-naphthyl)-L-alanine) | | |
| 146 | (D-4-pyridyl-alanine)-(L-3-pyridyl-alanine)-(D-Abu(tetrazol-5-yl))-(Tyr(3-5- | 7.5 | 7.3 |
| | di-Iodo))-(3-(4-Quinolyl))-L-alanine) | | |
| 147 | (D-Allothreonine)-(Beta-(4-thiazolyl)-Ala)-(D-Glu)-(Nme-Tyr(3-Iodo))-(L-2- | 5.9 | — |
| | indanylglycine) | | |
| 148 | (D-Allothreonine)-(Beta-(4-thiazolyl)-Ala)-(D-Glu)-(L-4- | 5.8 | 5.6 |
| | Benzoylphenylalanine)-((1-naphthyl)-L-alanine) | | |
| 149 | (D-Allothreonine)-(Beta-(4-thiazolyl)-Ala)-(D-Glu)-(D-His(1-Bn))-((1- | 5.3 | — |
| | naphthyl)-L-alanine) | | |
| 150 | (D-Glutamine)-(L-3-pyridyl-alanine)-(D-Abu(tetrazol-5-yl))-(Tyr(3-5-di- | 7.2 | 7.3 |
| | Iodo))-(3-(4-Quinolyl))-L-alanine) | | |
| 151 | (D-Allothreonine)-(L-3-pyridyl-alanine)-(D-Abu(tetrazol-5-yl))-(Tyr(3-5-di- | 6.7 | 7.5 |
| | Iodo))-(L-(7-azaTrp)) | | |
| 152 | (D-4-pyridyl-alanine)-(L-3-pyridyl-alanine)-(D-Abu(tetrazol-5-yl))-(Tyr(3-5- | 7.4 | 7.5 |
| | di-Iodo))-(L-(7-azaTrp)) | | |
| 153 | (D-Ala)-(L-3-pyridyl-alanine)-(D-Abu(tetrazol-5-yl))-(Tyr(3-5-di-Iodo))-(3- | 7.2 | 7.8 |
| | (4-Quinolyl))-L-alanine) | | |
| 154 | (D-Ala)-(L-3-pyridyl-alanine)-(D-Abu(tetrazol-5-yl))-(5-Bromo-2-fluoro-L- | 7.5 | 6.9 |
| | phenylalanine)-(2-(trifluoromethyl)-L-phenylalanine) | | |
| 155 | (D-Ala)-(L-3-pyridyl-alanine)-(D-Abu(tetrazol-5-yl))-(2-(trifluoromethoxy)- | 7.5 | 7.1 |
| | L-phenylalanine)-(2-(trifluoromethyl)-L-phenylalanine) | | |
| 156 | (D-Ala)-(L-3-pyridyl-alanine)-(D-Abu(tetrazol-5-yl))-(3-3-diphenyl-L- | 7.7 | 7.1 |
| | alanine)-(2-(trifluoromethyl)-L-phenylalanine) | | |
| 157 | (D-Ala)-(L-3-pyridyl-alanine)-(D-Abu(tetrazol-5-yl))-(3-3-diphenyl-L- | 7.9 | 7.3 |
| | alanine)-(L-2-indanylglycine) | | |
| 158 | (D-Allothreonine)-(L-3-pyridyl-alanine)-(D-Glu)-(3-3-diphenyl-L-alanine)- | 7.0 | 6.0 |
| | (2-(trifluoromethyl)-L-phenylalanine) | | |
| 159 | [*] (Propylamine)-(MPAA)-(D-Abu(tetrazol-5-yl)-(Tyr(3-5-di-Iodo))-(1- | 5.2 | — |
| | naphthyl)-L-alanine) | | |
| 160 | (D-Ala)-(3-5-difluoro-phenylalanine)-(D-Glu)-(homoTyr)-(L-2- | 6.4 | 6.1 |
| | indanylglycine) | | |
| 161 | (D-Ala)-(3-5-difluoro-phenylalanine)-(D-Ser(Ac))-(Tyr(3-5-di-Iodo))-(L-2- | 5.7 | 5.1 |
| | indanylglycine) | | |
| 162 | (D-Ala)-(3-5-difluoro-phenylalanine)-(3-nitro-D-phenylalanine)-(Tyr(3-5-di- | 5.3 | — |
| | Iodo))-(L-2-indanylglycine) | | |
| 163 | (D-Allothreonine)-(L-3-pyridyl-alanine)-(D-Abu(tetrazol-5-yl))-(Phe(3-5-di- | 7.5 | 7.1 |
| | Br))-(1-naphthyl)-L-alanine) | | |
| 164 | (D-Ala)-(L-3-pyridyl-alanine)-(D-Glu)-(2-Amino-3,3-bis(4- | 5.4 | — |
| | fluorophenyl)propanoic acid)-(2-(trifluoromethyl)-L-phenylalanine) | | |
| 165 | (D-Allothreonine)-(3-5-difluoro-phenylalanine)-(D-Glu)-(3-3-diphenyl-L- | 5.8 | 5.7 |
| | alanine)-(L-2-indanylglycine) | | |
| 166 | COO-(D-Ala)-(3-hydroxy-phenylalanine)-(D-Glu)-(2-(trifluoromethoxy)-L- | 6.2 | 5.7 |
| | phenylalanine)-(L-2-indanylglycine) | | |
| 167 | (D-Ala)-(3-5-difluoro-phenylalanine)-(D-Glu)-(Nva(Ph))-(L-2- | 6.6 | 6.9 |
| | indanylglycine) | | |
| 168 | (D-Ala)-(L-3-pyridyl-alanine)-(D-Glu)-(5-Bromo-2-fluoro-L-phenylalanine)- | 7.2 | 7.1 |
| | (2-(trifluoromethyl)-L-phenylalanine) | | |
| 169 | (D-Alanine)-(L-3-pyridyl-alanine)-(D-Cys(-Methyl-2H-tetrazole))-(Tyr(3-5- | 7.6 | 7.4 |
| | di-Iodo))-(L-2-indanylglycine) | | |
| 170 | (D-Ala)-(3-hydroxy-phenylalanine)-(D-Glu)-(L-Phe(2-OCF₃)))-(Nal) | 6.3 | 6.2 |
| 171 | (D-Ala)-(L-3-pyridyl-alanine)-(D-Abu(tetrazol-5-yl))-(2-methoxy-L- | 7.3 | 7.0 |
| | phenylalanine)-(2-(trifluoromethyl)-L-phenylalanine) | | |
| 172 | (D-Ala)-(3-hydroxy-phenylalanine)-(D-Glu)-(Phg(2-OMe)))-(Nal) | 5.4 | 4.8 |
| 173 | (D-Ala)-(L-3-pyridyl-alanine)-(D-Abu(tetrazol-5-yl))-(5-Bromo-2-fluoro-L- | 6.8 | 5.8 |
| | phenylalanine)-(2-(trifluoromethoxy)-L-phenylalanine) | | |
| 174 | (D-Cyclopropylglycine)-(L-3-pyridyl-alanine)-(D-Abu(tetrazol-5-yl))-(5- | 7.3 | 7.4 |
| | Bromo-2-fluoro-L-phenylalanine)-(2-(trifluoromethyl)-L-phenylalanine) | | |
| 175 | (D-Allothreonine)-(L-3-pyridyl-alanine)-(D-Abu(tetrazol-5-yl))-(5-Bromo-2- | 7.0 | 6.6 |
| | fluoro-L-phenylalanine)-(2-(trifluoromethyl)-L-phenylalanine) | | |
| 176 | (D-Ala)-(L-3-pyridyl-alanine)-(D-Abu(tetrazol-5-yl))-(2-(trifluoromethoxy)- | 7.3 | 7.1 |
| | L-phenylalanine)-(L-2-indanylglycine) | | |
| 177 | (D-Allothreonine)-(L-3-pyridyl-alanine)-(D-Abu(tetrazol-5-yl))-(2- | 7.4 | 7.4 |
| | (trifluoromethoxy)-L-phenylalanine)-(2-(trifluoromethyl)-L-phenylalanine) | | |
| 178 | (D-Gln)-(L-3-pyridyl-alanine)-(D-Abu(tetrazol-5-yl))-(2-(trifluoromethoxy)- | 7.5 | 7.4 |
| | L-phenylalanine)-(2-(trifluoromethyl)-L-phenylalanine) | | |
| 179 | (D-Allothreonine)(L-3-pyridyl-alanine)(gamma-Carboxy-D-glutamic-acid)(5- | 5.3 | — |
| | bromo-2-fluoro-L-phenylalanine)(2-(trifluoromethoxy)-L-phenylalanine) | | |
| 180 | (D-Glutamine)(L-3-pyridyl-alanine)(D-Abu(tetrazol-5-yl))(2- | 7.6 | 7.8 |
| | (trifluoromethoxy)-L-phenylalanine)(2-(trifluoromethoxy)-L-phenylalanine) | | |
| 181 | (D-Allothreonine)(L-3-pyridyl-alanine)(D-Abu(tetrazol-5-yl))(2-methoxy-L- | 6.5 | 6.4 |
| | phenylalanine)((S)-2-Methoxy-phenylglycine) | | |
| 182 | (D-Glutamine)(L-3-pyridyl-alanine)(D-Abu(tetrazol-5-yl))(2- | 6.3 | 5.3 |
| | (trifluoromethoxy)-L-phenylalanine)(L-2-Amino-3-guanidinopropionic-acid) | | |
| 183 | (D-Allothreonine)(L-3-pyridyl-alanine)(D-Abu(tetrazol-5-yl))(2- | 7.1 | 7.0 |
| | (trifluoromethoxy)-L-phenylalanine)(L-(7-azaTrp)) | | |
| 184 | (D-Glutamine)(3-pyridyl-alanine)(D-Abu(tetrazol-5-yl))(3-3-diphenyl- | 7.3 | 7.5 |
| | alanine)(2-(trifluoromethyl)-phenylalanine) | | |
| 185 | (D-Allothreonine)(L-3-pyridyl-alanine)(D-Abu(tetrazol-5-yl))(L-threo-3-Ph- | 7.2 | 6.5 |
| | Ser-OMe)(2-(trifluoromethyl)-L-phenylalanine) | | |
| 186 | (D-Allothreonine)(L-3-pyridyl-alanine)(D-Abu(tetrazol-5-yl))(2-methoxy-L- | 6.7 | 7.0 |
| | phenylalanine)(2-(trifluoromethyl)-L-phenylalanine) | | |
| 187 | (D-Allothreonine)(L-3-pyridyl-alanine)(D-Abu(tetrazol-5-yl))(2- | 6.9 | 6.6 |
| | (trifluoromethoxy)-L-phenylalanine)(L-Kynurenine) | | |
| 188 | DAla-Pal(3)-DAbu(tetrazol-5-yl)-homoTyr-Phe(2-CF₃) | 6.5 | — |

[*] peptidomimetic containing a thioether bond between first and second compounds
[—] assay not performed

In order to verify the pathway by which the inhibitory effect of the peptidomimetics is achieved, the interaction of the peptidomimetics with IL-17A, i.e., the dissociation constant thereof (K_{d}), was tested by Microscale Thermophoresis (MST) according to the protocol described in the “methods” section hereinabove. All 188 of the peptidomimetics presented a significant binding affinity to IL-17A, corroborating the AlphaLISA and ELISA results, and suggesting that the peptidomimetics inhibit IL-17A-IL-17R interaction at least by binding to IL-17A and diminishing its ability to interact with IL-17 receptor.

### Example 2: Inhibition of IL-17AF and IL-17F Interaction with IL-17R

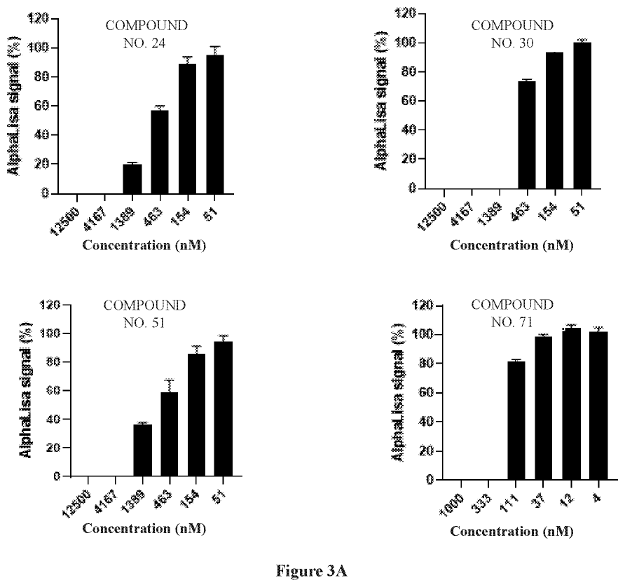

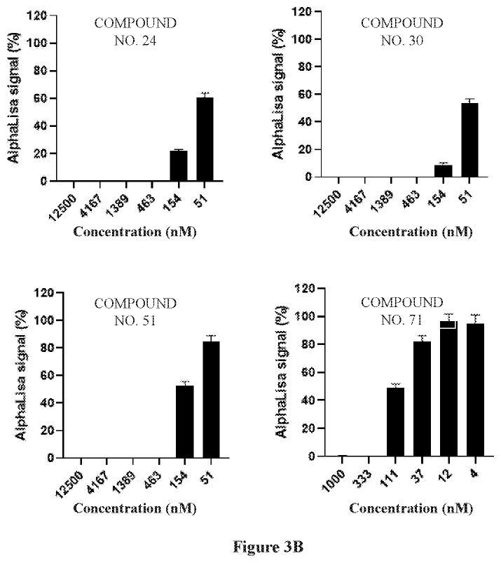

As homodimers of IL-17A and of IL-17F, as well as heterodimers of IL-17AF, are all capable of binding and activating the IL-17R, it was next tested whether the peptidomimetics which presented effective IL-17A binding, would also be effective at preventing IL-17F-IL-17R and IL-17AF-IL-17R interactions. AlphaLISA was performed for a representative group of peptidomimetics, selected from the 188 active cyclic penta-peptidomimetics, on IL-17F homodimers and IL-17AF heterodimers, according to the protocol hereinabove, and with each peptidomimetic measured at 6 different concentrations. Exemplary peptidomimetic results of the AlphaLISA for IL-17F and IL-17AF are presented in FIGS. 3A and 3B, respectively. The p(IC₅₀) values of each of the selected cyclic penta-peptidomimetics were then calculated based on the response curves of the different peptide concentrations and are set out in Table 3.

TABLE 3
In-vitro peptidomimetic IL-17F and IL-17AF inhibitory activity

| Compound | p(IC₅₀) | | |
|---|---|---|---|
| No. | IL-17A | IL-17F | IL-17AF |
| 24 | 5.7 | 7.1 | 6.4 |
| 30 | 7.1 | 7.1 | 6.1 |
| 31 | 7.0 | 7.1 | 6.5 |
| 35 | 6.8 | 6.8 | 6.3 |
| 51 | 6.9 | 6.9 | 6.3 |
| 57 | 6.2 | 6.4 | 6.4 |
| 68 | 5.8 | 6.3 | 5.8 |
| 71 | 6.4 | 6.8 | 6.7 |
| 78 | 7.5 | 7.3 | 7.1 |
| 81 | 6.6 | 6.5 | 6.1 |
| 88 | 6.8 | 6.4 | 6.3 |
| 91 | 6.9 | 6.6 | 6.3 |
| 92 | 7.6 | 6.9 | 6.8 |
| 93 | 7.4 | 6.3 | 6.6 |
| 114 | 7.1 | 7.0 | — |
| 117 | 7.3 | 7.3 | — |
| 121 | 7.5 | 6.7 | 7.7 |
| 123 | 7.6 | 7.4 | — |
| 125 | 7.7 | 7.1 | — |
| 126 | 7.1 | 6.9 | — |
| 127 | 7.9 | 7.4 | 7.4 |
| 128 | 6.8 | 6.3 | 6.0 |
| 134 | 7.7 | 7.5 | — |
| 135 | 7.4 | 7.0 | — |
| 140 | 7.6 | 6.8 | 6.5 |
| 150 | 7.2 | 7.0 | 6.6 |
| 151 | 6.7 | 6.7 | 6.5 |
| 152 | 7.4 | 0 | 0 |
| 153 | 7.2 | 0 | 6.5 |
| 154 | 7.5 | 7.1 | 7.6 |
| 155 | 7.5 | 6.6 | 7.1 |
| 156 | 7.7 | 6.7 | 7.1 |
| 157 | 7.6 | 6.8 | 7.6 |

[—] assay not performed
[0] indicates a peptidomimetic that did not reach 50% inhibition at the maximal concentration
(~10 μM) and is hence presumed inactive.

As can be clearly seen from Table 2 above, the selected peptidomimetics were found to be potent at inhibiting the binding of IL-17A, IL-17F and IL-17AF to IL-17R. It is noted however, that Compound 152 is specific to IL-17A while Compound No. 153 is specific to IL-17A and IL-17AF without activity on IL-17F.

### Example 3: Inhibition of IL-17-IL-17R Interaction in Keratinocytes Cell Culture

Following establishment of the inhibitory effectivity of the 188 peptidomimetics under in vitro conditions, the peptidomimetics were further examined for their ability to alter cellular activity by preventing activation of the IL-17 receptor in cell culture. Primary keratinocytes were cultured in a 384 well plate with 3,500 cells per well in a serum-free medium (C-20021, Promocell). 24 h after seeding, cyclic penta-peptidomimetics were added to the cells, at 7 different concentrations. Following the addition of the peptides, cells were treated with Human IL-17A (ILA-H82Q1, 3 ng/ml final concentration) and TNF-α (TNA-H4211, 10 ng/ml final concentration), or with TNF-α alone. Incubation with TNF-α alone served as a background for the synergistic activation of IL-17A and TNF-α. Following 48 h of incubation, culture medium was collected from the cells and analyzed for IL-8 levels (as a readout for IL-17 activation) using an AlphaLISA kit (AL224F, Perkin Elmer). Percent of inhibition was determined by fitting the calculated signal values to a standard 4-parameter logistic and non-linear regression analysis was performed using Prism software.

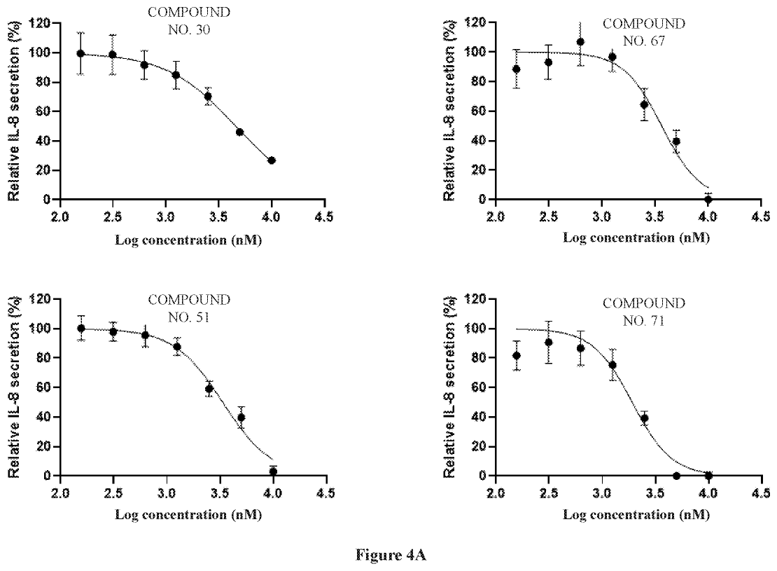

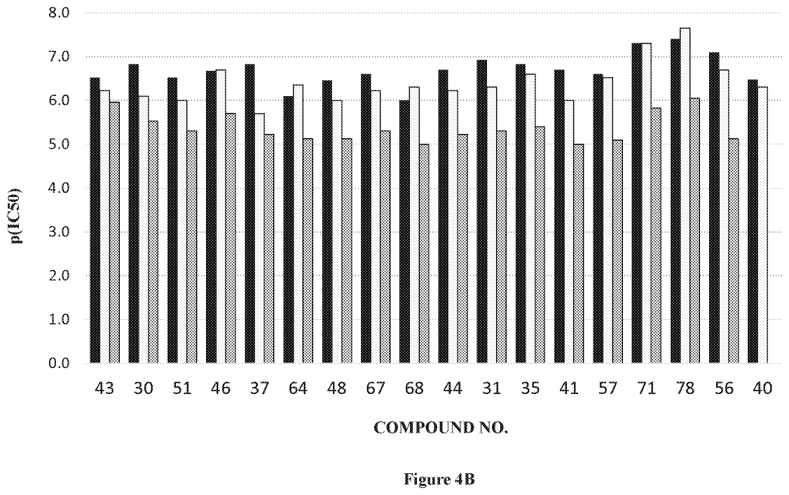

Graphs representing the IL-8 level reduction as a function of the rise in concentration in the cell culture medium of exemplary peptidomimetics are shown in FIGS. 4A and 9B. The p(IC₅₀) values of each of the examined peptidomimetic, calculated based on the IL-8 reduction curves at the different peptide concentrations, are set out in Table 3. FIG. 4B presents a graph comparing the p(IC₅₀) values of each peptidomimetic, as measured and calculated in AlphaLISA (black bar), ELISA (white bar), and cell culture (grey bar).

TABLE 3
Peptidomimetic IL-17A inhibitory activity in cells

| Compound No. | pIC₅₀ Cells |
|---|---|
| 30 | 5.5 |
| 31 | 5.3 |
| 35 | 5.4 |
| 37 | 5.2 |
| 41 | 5.2 |
| 43 | 5.6 |
| 44 | 5.2 |
| 46 | 5.5 |
| 48 | 5.2 |
| 51 | 5.3 |
| 56 | 5.4 |
| 57 | 5.1 |
| 64 | 5.2 |
| 65 | 5.2 |
| 66 | 5.3 |
| 67 | 5.4 |
| 68 | 5.2 |
| 71 | 5.9 |
| 78 | 6.3 |
| 84 | 5.8 |
| 88 | 5.4 |
| 91 | 5.3 |
| 92 | 5.8 |
| 93 | 6.1 |
| 94 | 0 |
| 95 | 0 |
| 97 | 5.7 |
| 98 | 0 |
| 100 | 5.8 |
| 101 | 5.6 |
| 102 | 0 |
| 104 | 0 |
| 105 | 0 |
| 106 | 5.3 |
| 107 | 0 |
| 109 | 5.9 |
| 112 | 5.9 |
| 114 | 5.8 |
| 117 | 6.3 |
| 119 | 0 |
| 120 | 5.5 |
| 121 | 6.1 |
| 122 | 5.3 |
| 123 | 6.3 |
| 125 | 6.0 |
| 127 | 6.3 |
| 128 | 5.7 |
| 129 | 0 |
| 131 | 0 |
| 132 | 5.7 |
| 134 | 6.5 |
| 135 | 6.0 |
| 136 | 6.5 |
| 137 | 5.7 |
| 138 | 6.1 |
| 139 | 5.8 |
| 140 | 6.1 |
| 142 | 5.4 |
| 145 | 0 |
| 146 | 5.9 |
| 150 | 6.5 |
| 151 | 6.5 |
| 152 | 6.1 |
| 153 | 5.8 |
| 154 | 6.1 |
| 155 | 6.0 |
| 156 | 6.1 |
| 157 | 6.0 |
| 160 | 5.2 |
| 163 | 6.3 |
| 166 | 0 |
| 167 | 5.9 |
| 168 | 5.6 |
| 169 | 5.5 |
| 170 | 5.6 |
| 171 | 5.7 |
| 173 | 0 |
| 174 | 6.4 |
| 176 | 6.1 |
| 177 | 6.1 |
| 178 | 6.1 |
| 180 | 6.2 |
| 181 | 5.4 |
| 183 | 5.8 |
| 184 | 6.0 |
| 186 | 6.2 |

[0] indicates a peptidomimetic that did not reach 50% inhibition at the maximal concentration
(~10 μM) and is hence presumed inactive.

As is clearly shown in Table 3, the inhibitory activity of the examined peptidomimetics in cell culture was generally correlated with the level of inhibitory activity identified by AlphaLISA and ELISA, showing a small reduction in the p(IC₅₀) value of between about 0.4 to 1.5 relative thereto.

### Example 4: Treatment of Psoriasis In Vivo with IL-17A Inhibiting Peptidomimetic

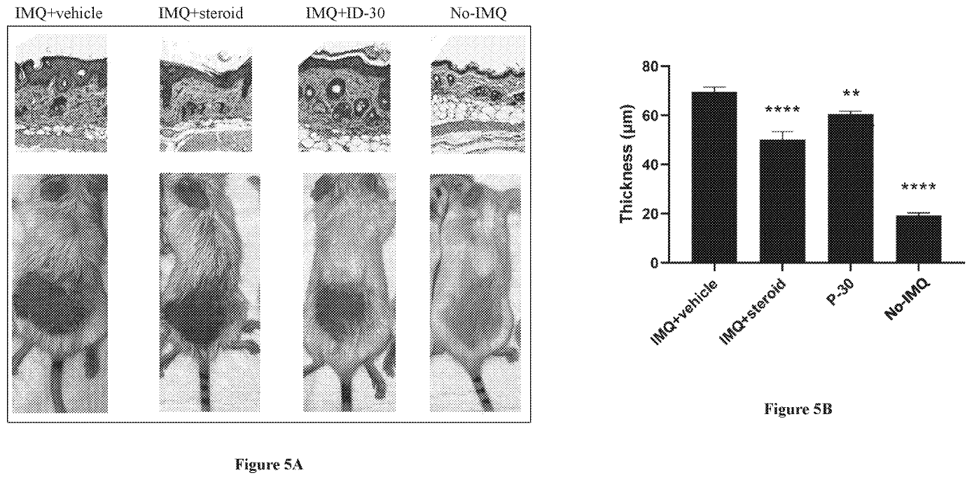

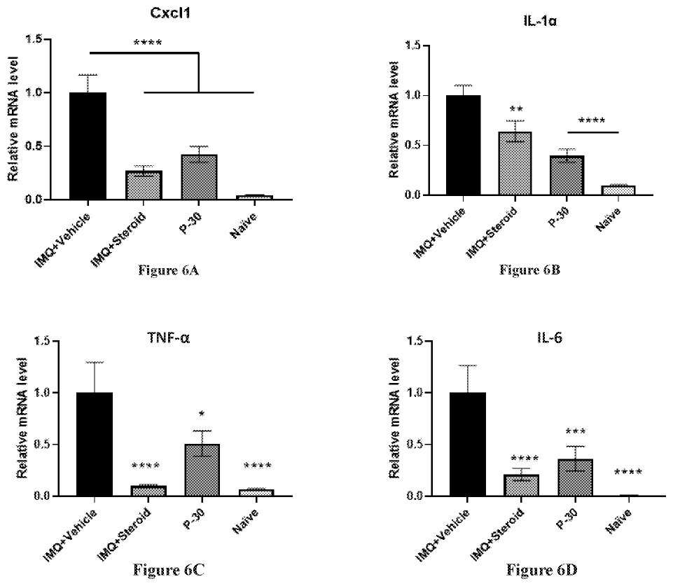

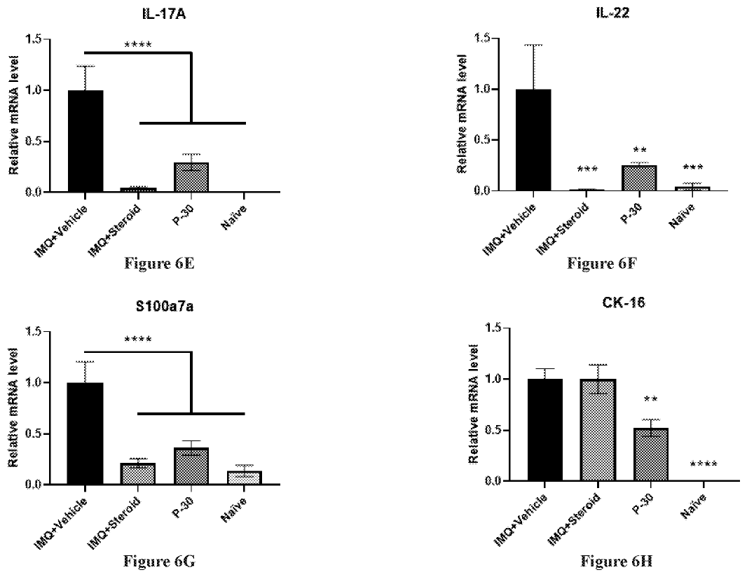

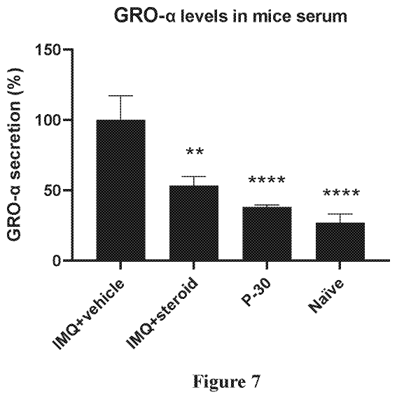

The therapeutic potential of the peptidomimetics in live animals was tested. An Imiquimod (IMQ) mouse psoriasis model was selected, which is well-validated with a high dependency on IL-17 for the pathologic outcome. Mice were divided into 4 groups (n-6), according to the respective treatment regimens, as follows: IMQ+DMSO (designated “IMQ+Vehicle”), as a negative control; IMQ+Daivobet (0.5 mg/g betamethasone), Diavobet being an approved drug for treatment of psoriasis (designated “IMQ+Steroid”); IMQ+peptidomimetic ID-30 (Compound No. 30), of the present invention (designated “IMQ+ID-30”); and an untreated control group (designated “no-IMQ”). On days 1-7 of the treatment, the mice groups received a daily dosage of IMQ (topical), and on days 4-7 the mice received topical administration of the respective treatment formulations twice-daily, i.e., DMSO, Daivobet, and DMSO+ID-30 (2% v/v), respectively. On day 8 the mice were sacrificed, and skin thickness (FIGS. 5A-5C), expression of genes related to IL-17 activation (FIGS. 6A-6H), and levels of GRO-Alpha protein in the mouse serum (encoded by the Cxcl1 gene) (FIG. 6I), were measured.

As is shown in the above-mentioned Figures, the mouse group treated with the peptidomimetic ID-30 showed significant improvement in all of the measured criteria relative to the IMQ+Vehicle mouse group (i.e., treated only with DMSO). Specifically, skin thickness was reduced by about 10 micrometers (about 12% reduction), gene expression was reduced by between about 50%-75%, and GRO-Alpha was reduced in the serum by about 70%. Strikingly, the reduction of GRO-Alpha was even more significant than that achieved by the formulated and approved Daivobet drug, as also was the decrease in the expression levels of the genes IL-1-alpha and CK-16.

### Example 5: Comparison of IL-17 Inhibitory Activity Between Exemplary Peptidomimetic and Closely-Related Peptidomimetic

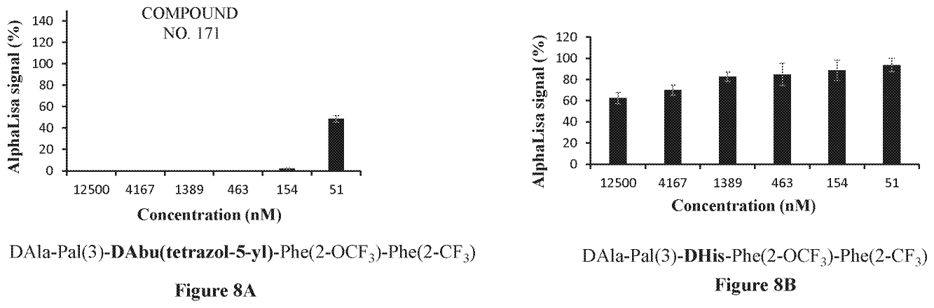

In order to demonstrate the accuracy of the molecular model and algorithm of the present invention, the residue at position 3 of Compound No. 171, which is the heteroaromatic D-Abu(tetrazol-5-yl) that provides a cluster of negative atomic partial charges, was replaced by a 5-membered heteroaromatic D-histidine, which contains an imidazole side chain. The imidazole is partially protonated and hence, carries a positive charge. According to the molecular model of some embodiments of the present invention, position 3 of the cyclic pentapeptide is required to have an anionic residue and/or a cluster of negative atomic partial charges at physiological pH, as is exemplified by Compound No. 171. The ability of each compound to inhibit IL-17A was then tested using AlphaLISA, as described in the methods section hereinabove. As demonstrated in FIGS. 8A and 8B, a dramatic difference in the activity of the two peptidomimetics to inhibit the binding of IL-17A to IL-17R can be seen. This unequivocally demonstrates the accuracy of the molecular model and the superiority of the peptidomimetics of the present invention.

### Example 6: Permeability Studies

The intestinal permeability of the peptides was studied by evaluating the permeability coefficient (Pₐₚₚ) of each compound from the apical to the basolateral membrane (PₐₚₚAB) as well as the Pₐₚₚ from the basolateral to the apical membrane (PₐₚₚBA) in a Caco-2 model.

Caco-2 cells (ATCC, Manassas, VA, USA) were grown in 75 cm² flasks with approximately 0.5×10⁶ cells/flask (Thermo-Fischer, Waltham, MA, USA) at 37° C. in a 5% CO₂ atmosphere and at a relative humidity of 95%. The culture growth medium consisted of DMEM supplemented with 10% heat-inactivated FBS, 1% MEM-NEAA, 2 mM L-glutamine, 1 mM sodium pyruvate, 50,000 units of penicillin G sodium, and 50 mg of streptomycin sulfate (Biological Industries, Israel). The medium was replaced every other day (Hess, S. et al, J Med Chem. 2007 Nov. 29; 50 (24): 6201-11).

Cells were then seeded onto polyethylene membranes (PET) in 96-well Corning Insert plates at 1×10⁵ cells/cm2, and medium was refreshed every 4~5 days until to the 21st to 28th day for confluent cell monolayer formation. Transepithelial electrical resistance (TEER) values were measured by the Millicell ERS-2 system (Millipore, Burlington, MA, USA), a week after seeding up to experiment day (21-23 days) to ensure proliferation and differentiation of the cells. When the cells were fully differentiated and Transepithelial Electrical Resistance (TEER) values became stable (200-500 Ω·cm²), the TEER values were compared to the control inserts containing only the medium (Ovadia, O. et al., Mol Pharm. 2011 Apr. 4; 8(2): 479-87).

The intestinal permeability of the compounds was studied by evaluating the permeability coefficient (Pₐₚₚ) of each compound from the apical to the basolateral membrane (PₐₚₚAB) as well as the Pₐₚₚ from the basolateral to the apical membrane (PₐₚₚBA) in a Caco-2 model.

The transport buffer in the study was HBSS with 10.0 mM HEPES at pH 7.40+0.05. Test compound was tested at 2 μM bi-directionally in duplicate. Digoxin was tested at 10.0 μM bi-directionally in duplicate, while nadolol and metoprolol were tested at 2.00 μM in A to B direction in duplicate. Final DMSO concentration was adjusted to less than 1%. The plate was incubated for 2 hours in CO₂ incubator at 37±1° C., with 5% CO₂ at saturated humidity without shaking. And all samples after mixed with acetonitrile containing internal standard were centrifuged at 3200×g for 10 min. For nadolol and metoprolol, 200 μL supernatant solution was diluted with 600 μL ultra-pure water for LC-MS/MS analysis. For digoxin and test compounds, 200 μL supernatant solution was diluted with 200 μL ultra-pure water for LC-MS/MS analysis. Concentrations of test and control compounds in starting solution, donor solution, and receiver solution were quantified by LC-MS/MS methodologies, using peak area ratio of analyte/internal standard. After transport assay, lucifer yellow rejection assay was applied to determine the Caco-2 cell monolayer integrity.

The apparent permeability coefficient Pₐₚₚ (cm/s) was calculated using the equation:

$${P}_{app} = ( dCr / dt ) \times Vr / ( A \times C 0 )$$

Where dCr/dt is the cumulative concentration of compound in the receiver chamber as a function of time (μM/s); Vr is the solution volume in the receiver chamber (0.075 mL on the apical side, 0.25 mL on the basolateral side); A is the surface area for the transport, i.e. 0.0804 cm² for the area of the monolayer; C0 is the initial concentration in the donor chamber (μM).

The efflux ratio was calculated using the equation:

$$Efflux Ratio = {P}_{app} ( BA ) / {P}_{app} ( AB )$$

Percent recovery was calculated using the equation:

$$\% Solution Recovery = 100 \times ( Vr \times Cr ) + ( Vd \times Cd ) ] / ( Vd \times C 0 )$$

Where Vd is the volume in the donor chambers (0.075 mL on the apical side, 0.25 mL on the basolateral side); Cd and Cr are the final concentrations of transport compound in donor and receiver chambers, respectively.

1. A cyclic penta-peptidomimetic that inhibits the interaction between an interleukin 17 (IL-17) and an IL-17 receptor (IL-17R), wherein:
the amino acid at position 1, counted from the N-terminus of the pentapeptide before cyclization, is selected from the group consisting of D-Ala, D-aThr, D-Pal(4), D-Gln, D-Abu, D-Gly(cPr), and D-Nle(Me);
the amino acid at position 2 is selected from the group consisting of Pal(3), Phe(3-OEt), Phe(3-OMe), and Phe(3-5-di-F);
the amino acid at position 3 is selected from the group consisting of D-Abu(tetrazol-5-yl), D-Cys(-Methyl-2H-tetrazole)), and D-Glu;
the amino acid at position 4 is selected from the group consisting of Tyr(3-5-di-Iodo), 5-Bromo-2-fluoro-phenylalanine, Phe(2-OCF₃), Dip, Phe(2-OMe), Phe (bR-OH), and Phe(3-5-di-Br); and
the amino acid at position 5 is selected from the group consisting of Gly (indan-2-yl), Phe(2-CF₃), 1-Nal, 3-(4-Quinolyl)-alanine, L-(7-azaTrp), Phe(2-OCF₃), and 2-Nal.

2. The cyclic penta-peptidomimetic according to claim 1, selected from Compound Nos. 125, 134, 127, 123, 78, 157, 178, 169, 153, 140, 136, 121, 152, 156, 146, 174, 177, 176, 92, 135, 155, 163, 150, 154, 173, 93, 171, 117, 168, 151, 101, 137, 114, 175, 180, 183, 184, 185, and 30.

3. The cyclic penta-peptidomimetic according to claim 1, comprising an amino acid selected from D-Ala, D-aThr, and D-Pal(4) at position 1; the amino acid Pal(3) at position 2; an amino acid selected from D-Abu(tetrazol-5-yl) and D-Glu at position 3; an amino acid selected from Tyr(3-5-di-Iodo), 5-Bromo-2-fluoro-L-phenylalanine, and L-Phe(2-OCF₃) at position 4; and an amino acid selected from Gly (indan-2-yl), Phe(2-CF₃), 1-Nal, and 3-(4-Quinolyl)-alanine at position 5.

4. The cyclic penta-peptidomimetic according to claim 1, selected from Compound Nos. 125, 78, 123, 127, 134, 136, 137, 140, 146, 153, 154, 155, 168, 175, 176, 177, 109, 41, 64, and 47.

5. The cyclic penta-peptidomimetic according to claim 1, having the structure:

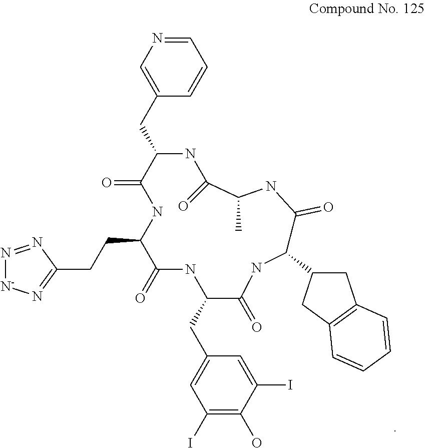 [SMILES: C[C@H]1NC(=O)[C@H](C2Cc3ccccc3C2)NC(=O)[C@H](Cc2cc(I)c(O)c(I)c2)NC(=O)[C@@H](CCc2nn[n-]n2)NC(=O)[C@H](Cc2cccnc2)NC1=O]

6. The cyclic penta-peptidomimetic according to claim 1, having an IL-17 inhibitory activity of p(IC₅₀)=7.0 or above, as determined by AlphaLISA and/or an IL-17 inhibitory activity of p(IC₅₀) of 5.5 or above, as determined by activation of IL-8.

7. The cyclic penta-peptidomimetic according claim 1, wherein the cyclic penta-peptidomimetic is capable of inhibiting the interaction between an IL-17A homodimer and an IL-17R, an IL-17F homodimer and an IL-17R, and/or IL-17AF heterodimer and an IL-17R.

8. A pharmaceutical composition comprising at least one cyclic penta-peptidomimetic according to claim 1, and at least one excipient, salt or carrier.

9. The pharmaceutical composition according to claim 8, wherein the pharmaceutical composition is formulated for local, parenteral or enteral administration.

10. The pharmaceutical composition according to claim 8, wherein the pharmaceutical composition is formulated for administration by injection, infusion, inhalation, as a nasal spray, for intradermal administration, subcutaneous administration, for administration locally to the dermis or for administration by electroporation.

11. A method for treating a patient suffering from an IL-17 associated disease, condition, or disorder, comprising administering to a patient in need thereof an effective amount of at least one cyclic pentapeptide according to claim 1, or a pharmaceutically acceptable salt thereof.

12. The method according to claim 11, further comprising providing at least one additional treatment selected from an anti-inflammatory agent or therapy and an immunosuppressant.

13. The method according to claim 11, wherein the IL-17 associated disease, condition, or disorder, is selected from the group consisting of psoriasis, psoriatic arthritis rheumatoid arthritis, spondyloarthritis, multiple sclerosis, axial spondyloarthritis, ankylosing spondylitis, hidradenitis suppurativa, systemic lupus erythematosus, palmoplantar pustulosis (PPP), atopic dermatitis, asthma, inflammatory bowel disease, COPD, age-associated skin condition, and allograft rejection.

14. A dosage form suitable for administration locally or by injection, infusion, inhalation or as a nasal spray, wherein the dosage form comprises at least one package which contains the pharmaceutical composition according to claim 8.

15. A head-to-tail cyclic penta-peptidomimetic that inhibits the interaction between an interleukin 17 (IL-17) and an IL-17 receptor (IL-17R), wherein the head-to-tail cyclic penta-peptidomimetic is selected from the group consisting of Compound Nos. 1 to 188.

16. A pharmaceutical composition comprising at least one cyclic penta-peptidomimetic according to claim 15, and at least one excipient, salt or carrier.

17. A method for treating a patient suffering from an IL-17 associated disease, condition, or disorder, comprising administering to a patient in need thereof an effective amount of at least one cyclic pentapeptide according to claim 15, or a pharmaceutically acceptable salt thereof.

18. A cosmetic composition comprising at least one cyclic penta-peptidomimetic according to claim 1, and at least one cosmetically acceptable excipient, salt or carrier.

19. The cosmetic composition according to claim 18, wherein the cosmetic composition is formulated as a lotion, a cream, an ointment, a paste, a gel, a hydrogel, a powder, a solution, a mist, or a spray.

20. A method for preventing, treating, or alleviating a cosmetic condition or disorder, the method comprising administering an effective amount of a compound according to claim 1, or a cosmetically acceptable salt thereof.
